(19) United States
(12) Patent Application Publication
(10) Pub. No.: US 2024/0189453 A1
(43) Pub. Date: Jun. 13, 2024

(54) MATERIALS AND METHODS FOR NEUROFIBROMIN 2/MERLIN (NF2) GENE THERAPY

(71) Applicant: RESEARCH INSTITUTE AT NATIONWIDE CHILDREN'S HOSPITAL, Columbus, OH (US)

(72) Inventors: Long-Sheng Chang, Columbus, OH (US); Kevin Flanigan, Columbus, OH (US); Shibi Likhite, Columbus, OH (US); Kathrin Meyer, Columbus, OH (US)

(21) Appl. No.: 18/286,923

(22) PCT Filed: Apr. 13, 2022
(86) PCT No.: PCT/US22/24680

§ 371 Date: Oct. 13, 2023

Related U.S. Application Data
(60) Provisional application No. 63/174,803, filed on Apr. 14, 2021.

(51) Int. Cl. A61K 48/00; A61K 9/00; A61K 38/00; A61P 25/00; A61P 35/00; C07K 14/47; C12N 15/86

(52) U.S. Cl. CPC A61K 48/005; A61K 9/0019; A61K 9/0085; A61P 25/00; A61P 35/00; C07K 14/4716; C12N 15/86; A61K 38/00; C12N 2750/14143

(57) ABSTRACT

The present disclosure relates to methods of treating conditions associated with a need for Merlin protein, for example due to a defective Neurofibromin 2/Merlin (NF2) gene as in neurofibromatosis type 2 (NF2). In particular. the disclosure provides gene therapy vectors to specifically treat loss of expression of the Merlin protein or reduced Merlin protein levels.

This application claims priority to U.S. Provisional Patent Application No. 63/174,803, filed on Apr. 14, 2021, which is incorporated by reference herein in its entirety.

### FIELD

The present disclosure relates to methods of treating conditions associated with a need for Merlin protein, for example due to a defective Neurofibromin 2/Merlin (NF2) gene as in neurofibromatosis type 2 (NF2). In particular, the disclosure provides gene therapy vectors to specifically treat loss of expression of the Merlin protein or reduced Merlin protein levels.

### INCORPORATION BY REFERENCE OF THE SEQUENCE LISTING

This application contains, as a separate part of disclosure, a Sequence Listing in computer-readable form (Filename: 54446_SeqListing.txt; 79,842 bytes—ASCII text file created Apr. 13, 2022) which is incorporated by reference herein in its entirety.

### BACKGROUND

Neurofibromatosis type 2 (NF2) is an autologous dominant genetic disorder affecting 1 in 40,000 people worldwide that originates from the loss of the NF2 gene. The NF2 gene encodes the moesin, ezrin, and radixin-like protein named “Merlin.” Merlin functions as a tumor suppressor in the tissues of the nervous systems. It functions in contact-dependent inhibition of cell proliferation. NF2 patients carry a loss-of-function mutation on one allele throughout the entire body, and tumor formation in the patients is associated with the loss of heterozygosity in the remaining NF2 allele. This loss is often due to a second hit mutation, but the loss of expression or reduced NF2 tumor suppressor protein levels can also be caused by epigenetic or post-translational modifications in the absence of a mutation in the NF2 gene.

NF2 is a severe debilitating condition characterized by bilateral vestibular schwannomas, along with other intracranial, intraspinal, and peripheral tumors such as multiple meningiomas, spinal schwannomas, and ependymomas. It results in increased morbidity and reduced life expectancy. Symptoms can appear in early childhood and worsen over time. Other than tumors, NF2 causes significant morbidities, including hearing loss, tinnitus (ringing ears), balance problems, facial paralysis and other cranial nerve dysfunction, cataract, seizure, and brainstem compression.

There is no cure for NF2, and current “gold standard” treatment options include surgery and radiotherapy. Surgical removal of tumors frequently causes nerve damage and results in hearing and vision impairment, as well as facial paralysis. Radiotherapy, although a less invasive option, is thought to result in a significant increase of subsequent malignant transformations and secondary malignancies in additional tissue types. Furthermore, due to the underlining genetic contributions to tumor formation, successful treatments are short-lived due to tumor regrowth and new tumors developing from other affected cells. Thus, complications, side effects, inefficiency in tumor control, and associated loss of neurological abilities make these treatments inadequate. The high emotional and financial costs of repeated tumor treatments without therapies that could really halt the disease condition from progressing underlines the urgent need of this patient population.

In addition to NF2-associated tumors, vestibular schwannomas and meningiomas can arise sporadically in the general population. Most, if not all, vestibular schwannomas carry inactivating NF2 mutations. Also, about half of sporadic meningiomas have NF2 loss.

There thus remains a need in the art for treatments for both NF2-associated and sporadic vestibular schwannomas and meningiomas, and other conditions, including cancers, in which there is a need for Merlin protein tumor suppressor activity.

### SUMMARY

The disclosure provides gene therapy vectors that express a functional Merlin protein (also sometimes referred to as “NF2 protein” herein). The gene therapy vectors are useful for delivering a transgene encoding a Merlin protein to a subject in need of Merlin protein tumor suppressor activity (i.e., activity that reduces the growth of tumors or formation of new tumors in vivo or in vitro).

The provided methods treat conditions involving reduced Merlin protein levels. Such conditions include, but are not limited to, NF2 as well cancers with reduced Merlin protein levels not due to pathologic mutations of the NF2 gene. Such conditions also include, but are not limited to, reduced Merlin protein tumor suppressor activity due to transcriptional or post-translational regulation of NF2.

The disclosure provides methods of treatment comprising administering the gene therapy vectors by direct injection to tumors, systemic delivery routes such as intravenous delivery, intrathecal delivery, or any other delivery method used to apply the vectors directly into a tumor or the cerebrospinal fluid. The treatment stops the growth of existing tumors, shrinks existing tumors, and/or reduces or prevents the formation of new tumors.

The gene therapy vectors are useful for delivering a transgene to cells with reduced Merlin protein levels in a subject. Cells include, but are not limited to, Schwann cells and Meningeal cells. Cells include, but are not limited to, tumor cells, for example, schwannomas, meningiomas and glioblastomas.

The gene therapy vector is, for example, AAV1, AAV2, AAV3, AAV4, AAV5, AAV6, AAV7, AAV8, AAV9, AAVRH10, AAVRH74, AAV11, AAV12, AAV13, AAVTT or Anc80, AAV7m8 and their derivatives. The gene therapy vector is, for example, AAV2, AAV5, AAV6, AAV9, AAV8 or AAV10. The gene therapy vector is, for example, AAV9, AAV8 or AAV10. The gene therapy vector is, for example, AAV2, AAV5 or AAV6.

The transgene in the gene therapy vector comprises, for example, the 2.3kb NF2 promoter or a NF2 promoter of reduced size, the CB promoter, the P546 promoter, the MBP promoter or the CMV promoter that drives the expression of a Merlin protein with tumor suppressor activity.

The gene therapy vector comprises, for example, the NF2/Merlin isoform cDNA lacking exon 16, referred to as the NF2 Isoform I herein. The Merlin protein encoded by the NF2 Isoform I possesses tumor suppressor activity.

The gene therapy vector comprises, as another example, a polynucleotide encoding a Merlin protein with a mutation disrupting a phosphorylation site of the Merlin protein while the Merlin protein retains tumor suppressor activity.

An exemplary gene therapy vector comprises in sequence an AAV2 internal repeat (ITR), an NF2 promoter, an SV40 intron, an NF2 polynucleotide encoding a protein with tumor suppressor activity, a synthetic Poly A signal and a second AAV2 ITR.

The gene therapy vector is administered to a subject in need thereof, for example, using intrathecal delivery and the subject is placed in the Trendelenburg position after administration of the gene therapy vector.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

FIG. 1 provides schematics of exemplary construct designs for NF2 gene therapy.

FIG. 2a-b shows NF2 promoter-dependent differential GFP expression in mammalian cells.

FIG. 3a-b shows NF2 promoter-dependent differential NF2 expression in mammalian cells.

FIG. 4 shows NF2 promoter-dependent differential GFP expression in vivo.

FIG. 5 shows NF2 promoter-dependent differential GFP expression in vestibular nuclei.

FIG. 6 shows NF2 promoter-dependent GFP expression in sciatic nerve.

FIG. 7 shows NF2 promoter dependent GFP expression in sciatic nerve.

FIG. 8 shows promoter-dependent GFP expression in sciatic nerve.

FIG. 9 shows promoter-dependent GFP expression in sciatic nerve.

FIG. 10 shows NF2 promoter-dependent differential expression of NF2 in vivo.

FIG. 11 shows NF2 promoter-dependent differential NF2 expression in vivo.

FIG. 12 shows NF2 promoter-dependent differential NF2 expression in vivo.

FIG. 13 shows NF2 promoter-dependent differential NF2 expression in vivo.

FIG. 14a-b shows NF2 promoter-dependent differential NF2 S518A expression in mammalian cells.

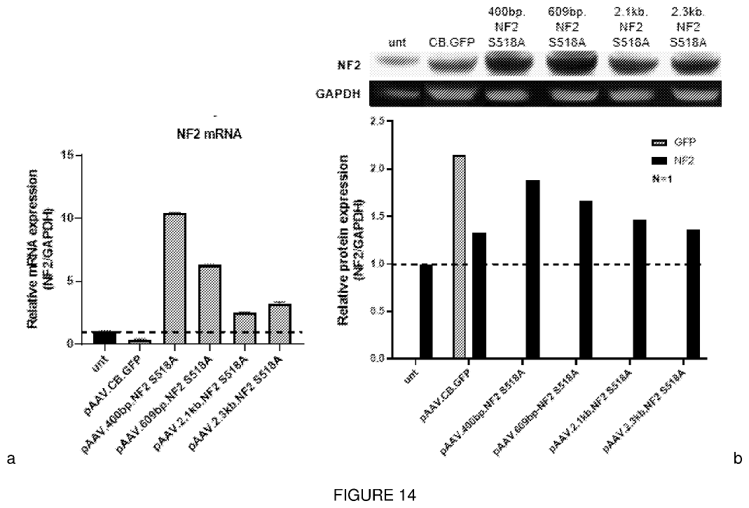

FIG. 15 shows that reprogrammed induced Schwann Cells (iSC) express Schwann cell markers (S100 and Krox20) nine (A) and 24 (B) days after reprogramming. Fibroblast Surface marker (FSM) is absent in both conditions.

FIG. 16 shows direct conversion of patient skin-derived fibroblasts to induced scwann cells (iSCs) to model NF2 disease in vitro.

FIG. 17 shows reduced NF2 expression in patient-derived human iSCs.

FIG. 18 shows reduced NF2 expression in patient-derived human iSCs.

FIG. 19 shows reduced NF2 expression in patient-derived human iSCs.

FIG. 20 shows increased Phosph-NF2 S518 expression in patient-derived human iSCs.

FIG. 21a-b shows reduced Schwann KROX20 and MBP cell maturation marker expression in NF2 patient iSCs.

FIG. 22a-b shows increased c-Myc stem cell marker expression in NF2 patient-derived iSCs.

FIG. 23a-b shows increased SOX2 stem cell marker expression in NF2 patient-derived iSCs.

FIG. 24 shows successful transduction of healthy control iSCs with AAV9.NF2.GFP vectors.

FIG. 25 shows successful transduction of NF2 patient-derived iSCs with AAV9.NF2.GFP vectors.

FIG. 26a-b shows reduction of c-Myc stem cell marker expression by AAV9.NF2 treatment in NF2 patient iSCs.

FIG. 27 shows reduction of SOX2 stem cell marker expression by AAV.NF2 treatment in NF2 patient iSCs.

FIG. 28a-d shows sorted iSCs directly hit with shNF2 have reduced NF2 levels.

FIG. 29a-c shows reduced NF2 expression in primary human Vestibular Schwannomas (hVS) tumor cells.

FIG. 30a-b shows increased Phospho-NF2 S518 expression in hVS tumor cells.

FIG. 31a-b shows hVS tumor cells are farther from mature Schwann cells.

FIG. 32a-b shows high proliferation rates in single-allele mutant NF2 patient ISCs and NF2 double knockdown patient iSCs.

FIG. 33a-b shows increased c-Myc and SOX2 stem cell marker expression in hVS tumor cells.

FIG. 34a-b shows reduction of c-Myc stem cell marker expression by AAV.NF2 treatment in hVS cells.

FIG. 35a-b shows reduction of SOX2 stem cell marker expression by AAV.NF2 treatment in hVS cells.

FIG. 36a-b shows reduced NF2 protein expression in glioblastomas.

FIG. 37a-b shows reduced NF2 mRNA expression in glioblastomas.

FIG. 38a-b shows AAV9.NF2 treatment restores NF2 protein expression in glioblastomas.

FIG. 39a-c shows AAV9.NF2 treatment restores NF2 mRNA expression in glioblastomas.

FIG. 40a-b shows phosphorylation resistant AAV9.NF2 treatment better restores NF2 protein in glioblastomas.

FIG. 41a-c shows phosphorylation resistant AAV9.NF2 treatment restores NF2 mRNA expression in glioblastomas.

FIG. 42a-b shows AAV9.NF2 treatment reduces cells proliferation in glioblastomas.

### DETAILED DESCRIPTION

### Gene Therapy Vectors

Adeno-associated virus (AAV) is a replication-deficient parvovirus, the single-stranded DNA genome of which is about 4.7 kb in length including two 145 nucleotide inverted terminal repeats (ITRs) and may be used to refer to the virus itself or derivatives thereof. The term covers all subtypes and both naturally occurring and recombinant forms, except where specified otherwise. There are multiple serotypes of AAV. The serotypes of AAV are each associated with a specific clade, the members of which share serologic and functional similarities. Thus, AAVs may also be referred to by the clade. For example, AAV9 sequences are referred to as “clade F” sequences (Gao et al., J. Virol., 78: 6381-6388 (2004). The present disclosure contemplates the use of any sequence within a specific clade, e.g., clade F. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. The sequence of the AAV rh.74 genome is provided in see U.S. Pat. No. 9,434,928, incorporated herein by reference. The sequence of the AAV-B1 genome is provided in Choudhury et al., Mol. Ther., 24(7): 1247-1257 (2016). Anc80 is an AAV vector that is of AAV1, AAV2, AAV8 and AAV9. The sequence of Anc80 is provided in Zinn et al., Cell Reports 12: 1056-1068, 2015 and Vandenberghe et al, PCT/US2014/060163, both of which are incorporated by reference herein, in their entirety and GenBank Accession Nos. KT235804-KT235812.

Cis-acting sequences directing viral DNA replication, encapsidation/packaging, and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The native AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. In some instances, the rep and cap proteins are provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

The term “AAV” as used herein refers to the wild type AAV virus or viral particles. The terms “AAV,” “AAV virus,” and “AAV viral particle” are used interchangeably herein. The term “rAAV” refers to recombinant, infectious, encapsulated virus or viral particles. The terms “rAAV,” “rAAV virus,” and “rAAV viral particle” are used interchangeably herein.

The term “rAAV genome” refers to a polynucleotide sequence that is derived from a native AAV genome that has been modified. rAAV genomes are provided that have been modified to remove the native AAV cap and rep genes. The rAAV genomes comprise at least one or both endogenous 5′ and 3′ inverted terminal repeats (ITRs). The rAAV genome can comprise ITRs from an AAV serotype that is different from the AAV serotype from which the AAV genome was derived.

rAAV genomes comprising a transgene flanked at the 5′ and 3′ ends by ITRs are provided herein.

SEQ ID NO: 1 sets out the polynucleotide sequence of the NF2/Merlin isoform lacking exon 16, referred to as the “NF2 Isoform I.” The NF2 Isoform I amino acid sequence encoded by SEQ ID NO: 1 is set out in SEQ ID NO: 15.

Transgenes provided herein include, but are not limited to, a transgene comprising the NF2 Isoform I polynucleotide of SEQ ID NO: 1, or a polynucleotide encoding a Merlin protein with tumor suppressor activity wherein the polynucleotide is 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the polynucleotide of SEQ ID NO: 1.

Transgenes provided herein include, but are not limited to, the transgenes set out in SEQ ID NOs: 6-9 which each comprise the NF2 Isoform I polynucleotide of SEQ ID NO: 1. Also provided herein are transgenes encoding a Merlin protein with tumor suppressor activity, wherein the transgenes are at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 6, 7, 8, or 9.

Transgenes provided herein can encode, for example, a Merlin protein with tumor suppressor activity that is at least about: 90%, 91%, 92%, 93%, 94%, 95%, 96%, 97%, 98%, or 99% identical to the NF2 Isoform I of SEQ ID NO: 15.

Transgenes provided herein include a polynucleotide that encodes a Merlin protein with tumor suppressor activity and that hybridizes under stringent conditions to a transgene comprising SEQ ID NO: 1 or to a transgene of SEQ ID NO: 6, 7, 8 or 9, or the complement thereof.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing include but are not limited to 0.015 M sodium chloride, 0.0015 M sodium citrate at 65-68° C. or 0.015 M sodium chloride, 0.0015M sodium citrate, and 50% formamide at 42° C. See, for example, Sambrook et al., Molecular Cloning: A Laboratory Manual, 2nd Ed., Cold Spring Harbor Laboratory, (Cold Spring Harbor, N.Y. 1989).

A Merlin protein with tumor suppressor activity can be resistant to post-translational negative regulation such as phosphorylation. Phosphorylation-resistant Merlin proteins with tumor suppressor activity provided herein include, but are not limited to, Merlin proteins in which a phosphorylation site or sites are removed by replacing one or more of the serine at position 10 of SEQ ID NO: 15, the threonine at position 230 of SEQ ID NO: 15, the serine at position 315 of SEQ ID NO: 15 and the serine at position 518 of SEQ ID NO: 15 with another amino acid such as alanine to prevent phosphorylation at/around that position. The amino acid replacement can be effected, for example, by changing the polynucleotide sequence encoding the Merlin protein. For example, changing the thymidine at position 1609 of SEQ ID NO: 1 to a guanosine results in the substitution of an alanine residue for a serine residue in the encoded Merlin protein. SEQ ID NO: 16 set outs a polynucleotide with a guanosine at that position so that encodes a phosphorylation-resistant Merlin protein. SEQ ID NOs: 17-20 set out transgenes including that polynucleotide encoding the phosphorylation-resistant Merlin protein.

Examples of promoters are the 2.3kb NF2 promoter (SEQ ID NO: 2), a truncated NF2 promoter [including but not limited to, a 400bp NF2 promoter (SEQ ID NO: 3), a 610bp NF2 promoter (sometime times referred to herein as 609bp) (SEQ ID NO: 4) and a 2.1kb NF2 promoter (SEQ ID NO: 5)], the CMV promoter, the chicken β actin promoter (CB), the P546 promoter and the Myo7A promoter. Additional promoters are contemplated herein including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Additionally provided herein are an NF2 promoter, truncated NF2 promoters, CMV promoter, CB promoter, P546 promoter, and Myo7A promoters at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the respectively corresponding native promoter nucleotide sequence, which possess transcription promoting activity.

Other examples of transcription control elements are tissue specific control elements, for example, promoters that allow expression specifically within neurons or specifically within astrocytes, or specifically within Schwann cells. Examples include neuron specific enolase, astrocyte-specific glial fibrillary acidic protein, and Schwann cell-specific myelin protein P0 promoters. Inducible promoters are also contemplated. Non-limiting examples of inducible promoters include, but are not limited to a metallothionine promoter, a glucocorticoid promoter, a progesterone promoter, and a tetracycline-regulated promoter. The gene cassette may also include intron sequences to facilitate processing of a transgene RNA transcript when expressed in mammalian cells. One example of such an intron is the SV40 intron.

“Packaging” refers to a series of intracellular events that result in the assembly and encapsidation of an AAV particle. The term “production” refers to the process of producing the rAAV (the infectious, encapsulated rAAV particles) by the packing cells.

AAV “rep” and “cap” genes refer to polynucleotide sequences encoding replication and encapsidation proteins, respectively, of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses may encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

The rAAV genomes provided herein lack AAV rep and cap DNA. AAV DNA in the rAAV genomes (e.g., ITRs) contemplated herein may be from any AAV serotype suitable for deriving a recombinant virus including, but not limited to, AAV serotypes Anc80, Anc80L65, AAV-1, AAV-2, AAV-3, AAV-4, AAV-5, AAV-6, AAV-7, AAV7mb, AAV-8, AAV-9, AAV-10, AAV-RH10, AAV-11, AAV-12, AAV-13, AAV rh.74 and AAV-B1 and their derivatives. As noted above, the nucleotide sequences of the genomes of various AAV serotypes are known in the art. rAAV with capsid mutations, are also contemplated. See, for example, Marsic et al., Molecular Therapy, 22(11): 1900-1909 (2014). Modified capsids herein are also contemplated and include capsids having various post-translational modifications such as glycosylation and deamidation. Deamidation of asparagine or glutamine side chains resulting in conversion of asparagine residues to aspartic acid or isoaspartic acid residues, and conversion of glutamine to glutamic acid or isoglutamic acid is contemplated in rAAV capsids provided herein. See, for example, Giles et al., Molecular Therapy, 26(12): 2848-2862 (2018). Modified capsids herein are also contemplated to comprise targeting sequences directing the rAAV to the affected tissues and organs requiring treatment.

DNA plasmids provided herein comprise rAAV genomes described herein. The DNA plasmids may be transferred to cells permissible for infection with a helper virus of AAV (e.g., adenovirus, E1-deleted adenovirus or herpesvirus) for assembly of the rAAV genome into infectious viral particles with AAV9 capsid proteins. Techniques to produce rAAV, in which an rAAV genome to be packaged, rep and cap genes, and helper virus functions are provided to a cell are standard in the art. Production of rAAV particles requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs. Production of pseudotyped rAAV is disclosed in, for example, WO 01/83692 which is incorporated by reference herein in its entirety. AAV capsid proteins may be modified to enhance delivery of the recombinant rAAV. Modifications to capsid proteins are generally known in the art. See, for example, US 2005/0053922 and US 2009/0202490, the disclosures of which are incorporated by reference herein in their entirety.

A method of generating a packaging cell is to create a cell line that stably expresses all the necessary components for rAAV production. For example, a plasmid (or multiple plasmids) comprising a rAAV genome lacking AAV rep and cap genes, AAV rep and cap genes separate from the rAAV genome, and a selectable marker, such as a neomycin resistance gene, may be integrated into the genome of a cell. rAAV genomes may be introduced into bacterial plasmids by procedures such as GC tailing [Samulski et al., Proc. Natl. Acad. S6. USA, 79:2077-2081 (1982)], addition of synthetic linkers containing restriction endonuclease cleavage sites [Laughlin et al., Gene, 23:65-73 (1983)] or by direct, blunt-end ligation [Senapathy and Carter, J. Biol. Chem., 259:4661-4666 (1984)]. The packaging cell line may then be infected with a helper virus such as adenovirus. The advantages of this method are that the cells are selectable and are suitable for large-scale production of rAAV. Other non-limiting examples of suitable methods employ adenovirus or baculovirus rather than plasmids to introduce rAAV genomes and/or rep and cap genes into packaging cells.

General principles of rAAV particle production are reviewed in, for example, Carter, Current Opinions in Biotechnology, 1533-539 (1992); and Muzyczka, Curr. Topics in Microbial. and Immunol., 158:97-129 (1992). Various approaches are described in Ratschin et al., Mol. Cell. Biol. 4:2072 (1984); Hermonat et al., Proc. Natl. Acad. Sci. USA, 81:6466 (1984); Tratschin et al., Mo1. Cell. Biol. 5:3251 (1985); Mclaughlin et al., J. Virol., 62:1963 (1988); and Lebkowski et al., Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776 ; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine, 13:1244-1250 1995); Paul et al., Human Gene Therapy, 4:609-615 (1993); Clark et al., Gene Therapy 3:1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV particle production.

Further provided herein are packaging cells that produce infectious rAAV particles. In one embodiment packaging cells may be stably transformed cancer cells such as HeLa cells, 293 cells and PerC.6 cells (a cognate 293 line). In another embodiment, packaging cells may be cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Also provided herein are rAAV (e.g., infectious encapsidated rAAV particles) comprising a rAAV genome of the disclosure. The genomes of the rAAV lack AAV rep and cap DNA, that is, there is no AAV rep or cap DNA between the ITRs of the genomes of the rAAV. The rAAV genome can be a self-complementary (sc) genome. A rAAV with a sc genome is referred to herein as a scAAV. The rAAV genome can be a single-stranded (ss) genome. A rAAV with a single-stranded genome is referred to herein as an ssAAV.

The rAAV may be purified by methods standard in the art such as by column chromatography or cesium chloride gradients. Methods for purifying rAAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

### Compositions

Compositions comprising rAAV are also provided. Compositions comprise a rAAV encoding a polypeptide of interest including, but not limited to, a Merlin protein. Compositions may include two or more rAAV encoding different polypeptides of interest.

Compositions provided herein comprise rAAV and a pharmaceutically acceptable excipient or excipients. Acceptable excipients are nontoxic to recipients and are preferably inert at the dosages and concentrations employed, and include, but are not limited to, buffers such as phosphate e.g., phosphate-buffered saline (PBS), citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, copolymers such as poloxamer 188, pluronics (e.g., Pluronic F68) or polyethylene glycol (PEG). Compositions provided herein can comprise a pharmaceutically acceptable aqueous excipient containing a non-ionic, low-osmolar compound or contrast agent such as iobitridol, iohexol, iomeprol, iopamidol, iopentol, iopromide, ioversol, or ioxilan, where the aqueous excipient containing the non-ionic, low-osmolar compound can have one or more of the following characteristics: about 180 mgl/mL, an osmolality by vapor-pressure osmometry of about 322 mOsm/kg water, an osmolarity of about 273 mOsm/L, an absolute viscosity of about 2.3 cp at 20° C. and about 1.5 cp at 37° C., and a specific gravity of about 1.164 at 37° C. Exemplary compositions comprise about 20 to 40% non-ionic, low-osmolar compound or about 25% to about 35% non-ionic, low-osmolar compound. An exemplary composition comprises scAAV or rAAV viral particles formulated in 20 mM Tris (pH8.0), 1 mM MgCl₂, 200 mM NaCl, 0.001% poloxamer 188 and about 25% to about 35% non-ionic, low-osmolar compound. Another exemplary composition comprises scAAV formulated in and 1×PBS and 0.001% Pluronic F68.

For CSF delivery including but not limited to intrathecal delivery, the viral vector can be mixed with a contrast agent (Omnipaque or similar). For example, the compositions may comprise a non-ionic, low-osmolar contrast agent including, but not limited to, iobitridol, iohexol, iomeprol, iopamidol, iopentol, iopromide, ioversol, ioxilan, or combinations thereof.

Dosages may be expressed in units of viral genomes (vg). Dosages contemplated herein include about 1×10⁷ vg, about 1×10⁸ vg, about 1×10⁹ vg, about 5×10⁹ vg, about 6×10⁹ vg, about 7×10⁹ vg, about 8×10⁹ vg, about 9×10⁹ vg, about 1×10¹⁰ vg, about 2×10¹⁰ vg, about 3×10¹⁰ vg, about 4×10¹⁰ vg, about 5×10¹⁰ vg, about 1×10¹¹ vg, about 1.1×10¹¹ vg, about 1.2×10¹¹ vg, about 1.3×10¹¹ vg, about 1.2×10¹¹ vg, about 1.3×10¹¹ vg, about 1.4×10¹¹ vg, about 1.5×10¹¹ vg, about 1.6×10¹¹ vg, about 1.7×10¹¹ vg, about 1.8×10¹¹ vg, about 1.9×10¹¹ vg, about 2×10¹¹ vg, about 3×10¹¹ vg, about 4×10¹¹ vg, about 5×10¹¹ vg, about 1×10¹² vg, about 1×10¹³ vg, about 1.1×10¹³ vg, about 1.2×10¹³ vg, about 1.3×10¹³ vg, about 1.5×10¹³ vg, about 2×10¹³ vg, about 2.5×10¹³ vg, about 3×10¹³ vg, about 3.5×10¹³ vg, about 4×10¹³ vg, about 4.5×10¹³ vg, about 5×10¹³ vg, about 6×10¹³ vg, about 1×10¹⁴ vg, about 2×10¹⁴ vg, about 3×10¹⁴ vg, about 4×10¹⁴ vg, about 5×10¹⁴ vg, about 1×10¹⁵ vg, to about 1×10¹⁶ vg, or more total viral genomes. Dosages of about 1×10⁹ vg to about 1×10¹⁰ vg, about 5×10⁹ vg to about 5 ×10¹⁰ vg, about 1×10¹⁰ vg to about 1×10¹¹ vg, about 1×10¹¹ vg to about 1×10¹⁵ vg, about 1×10¹² vg to about 1×10¹⁵ vg, about 1×10¹² vg to about 1×10¹⁴ vg, about 1×10¹³ vg to about 6×10¹⁴ vg, and about 6×10¹³ vg to about 1.0×10¹⁴ vg, 2.0×10¹⁴ vg, 3.0×10¹⁴ vg, 5.0×10¹⁴ are also contemplated. One dose exemplified herein is1.65×10¹¹ vg.

For example, CSF doses can range between about 1×10¹³ vg/patient to about 1×10¹⁵ vg/patient based on age groups. For example, intravenous delivery doses can range between 1×10¹³ vg/kilogram (kg) body weight and 2×10¹⁴ vg/kg.

### Methods of Treatment

Methods of treatment herein target cells with reduced Merlin protein tumor suppressor activity. Methods of treatment herein can target cells with a “defective” NF2 gene, that is a gene with at least one “defective” (i.e., mutated) allele encoding a Merlin protein that lacks tumor suppressor activity. As is understood in the art, a diploid subject such as a human subject generally has two copies of each gene which are referred to alleles. Methods of treatment herein can target cells with reduced Merlin protein tumor suppressor activity other than reduced activity caused by a defective NF2 gene, for example, reduced Merlin protein tumor suppressor activity caused by epigenic regulation or by post-translational modifications (e.g., phosphorylation) that reduce the Merlin protein tumor suppressor activity. Methods of transducing target cells in a subject (e.g., a human subject) are provided. Methods of transducing such Schwann and/or meningeal cells in a subject (e.g., a human subject) are provided. Method of transducing schwannomas, meningiomas and/or glioblastomas in a subject (e.g., a human subject) are provided.

Use of methods of treatment described herein is indicated for reduced Merlin protein tumor suppressor activity in, for example, NF2, acute myelogenous leukemia, squamous cell carcinoma, bladder cancer, sarcoma, breast cancer, ependymoma, colorectal carcinoma, mixed adenosquamous carcinoma, glioma, hepatocellular carcinoma, intestinal carcinoma, liver cancer, adenocarcinoma, mixed lung cancer, melanoma, meningioma, mesothelioma, serous carcinoma, pituitary carcinoma, renal cell carcinoma, Schwannoma (sporadic tumors in patients that do not have NF2), stomach cancer, anaplastic thyroid carcinoma, endometrial cancer, prostate cancer and urinary tract cancer.

The terms “transducing” and “transduction” are used to refer to the administration/delivery of rAAV of the disclosure encoding a Merlin protein with tumor suppressor activity to a target cell either in vivo or in vitro, resulting in expression of a functional Merlin protein by the target cell. Transduction of cells with rAAV of the disclosure results in sustained expression of polypeptide encoded by the rAAV.

Methods provided herein transduce target cells with one or more rAAV described herein. In some embodiments, the rAAV viral particle comprising a transgene is administered or delivered the brain and/or spinal cord of a subject by, for example, systemic administration (e.g., intravenous administration) or intrathecal administration. Intrathecal administration refers to delivery into the space under the arachnoid membrane of the brain or spinal cord. Intrathecal administration to the brain in particular can by carried out by intracerebroventricular injection. Areas of the brain contemplated for delivery include, but are not limited to, the motor cortex, visual cortex, cerebellum and the brain stem.

For intrathecal administration, the subject can be held in the Trendelenburg position (head down position) after injection of the rAAV (e.g., for about 5, about 10, about 15 or about 20 minutes). For example, the patient may be tilted in the head down position at about 1 degree to about 30 degrees, about 15 to about 30 degrees, about 30 to about 60 degrees, about 60 to about 90 degrees, or about 90 to about 180 degrees.

The treatment methods provided herein comprise the step of administering an effective dose, or effective multiple doses, of a composition comprising a rAAV provided herein to a subject (e.g., an animal including, but not limited to, a human patient) in need thereof. If the dose is administered prior to development of symptoms of NF2, the administration is prophylactic. If the dose is administered after the development of symptoms, the administration is therapeutic. An effective dose is a dose that alleviates (eliminates or reduces) at least one symptom associated with a condition, that slows or prevents progression of the condition, that diminishes the extent of the condition, that results in remission (partial or total) of the condition, and/or that prolongs survival.

An effective dose for treatment of schwannomas, meningiomas and/or glioblastomas is a dose that alleviates (eliminates or reduces) the schwannomas, meningiomas and/or glioblastomas with a defective NF2 gene, that slows or prevents development of the schwannomas, meningiomas and/or glioblastomas, that results in remission (partial or total) of the schwannomas, meningiomas and/or glioblastomas, and/or that prolongs survival.

An effective dose for treatment of NF2 is a dose that alleviates (eliminates or reduces) at least one symptom associated with NF2 [e.g., alleviates at least one of: tumors (reduces size and/or numbers of tumors, and/or reduces metastases forming a new tumor), hearing loss, tinnitus (ringing ears), balance problems, facial weakness or numbness, visual impairment, cataract, seizure, and brainstem compression], that slows or prevents progression of NF2, that diminishes the extent of NF2, that results in remission (partial or total) of NF2, and/or that prolongs survival.

The disclosure provides methods of treating hearing loss associated with NF2.

Test for determining whether a method of treatment described herein improves hearing loss or hearing impairment include physiological tests which objectively determine the functional status of the auditory system; and audiometry which subjectively determines how the individual processes auditory information. Physiological tests include the auditory brain stem response testing (ABR, also known as BAER, BSER), which uses a stimulus (e.g. clicks) to evoke electrophysiologic responses, which originate in the eighth cranial nerve and auditory brainstem and are recorded with surface electrodes. ABR “wave V detection threshold” correlates best with hearing sensitivity in the 1500- to 4000-Hz region in neurologically normal individuals; ABR does not assess low frequency (<1500 Hz) sensitivity.

Auditory steady-state response testing (ASSR) uses an objective, statistics-based mathematical detection algorithm to detect and define hearing thresholds. ASSR can be obtained using broadband or frequency-specific stimuli and can offer hearing threshold differentiation in the severe-to-profound range. The ASSR test is frequently used to give frequency-specific information that ABR does not give. Test frequencies of 500, 1000, 2000, and 4000 Hz are commonly used.

Evoked otoacoustic emissions (EOAEs) are sounds originating within the cochlea that are measured in the external auditory canal using a probe with a microphone and transducer. EOAEs reflect primarily the activity of the outer hair cells of the cochlea across a broad frequency range and are present in ears with hearing sensitivity better than 40-50 dB HL. Immittance testing (tympanometry, acoustic reflex thresholds, acoustic reflex decay) assesses the peripheral auditory system, including middle ear pressure, tympanic membrane mobility, Eustachian tube function, and mobility of the middle ear ossicles.

Audiometry includes behavioral tests such as behavioral observation audiometry (BOA) and visual reinforcement audiometry (VRA). Pure-tone audiometry (air and bone conduction) involves determination of the lowest intensity at which an individual “hears” a pure tone, as a function of frequency (or pitch). Octave frequencies from 250 (close to middle C) to 8000 Hz are tested using earphones. Intensity or loudness is measured in decibels (dB), defined as the ratio between two sound pressures. 0 dB HL is the average threshold for a normal hearing adult; 120 dB HL is so loud as to cause pain. Speech reception thresholds (SRTs) and speech discrimination are assessed. For air conduction audiometry sounds are presented through earphones; thresholds depend on the condition of the external ear canal, middle ear, and inner ear. For bone conduction audiometry sounds are presented through a vibrator placed on the mastoid bone or forehead, thus bypassing the external and middle ears; thresholds depend on the condition of the inner ear. Additional tests include conditioned play audiometry which develops a complete frequency-specific audiogram for each ear, and conventional audiometry which indicates when an individual hears a sound.

An audioprofile refers to the recording of several audiograms on a single graph. These audiograms may be from one individual at different times. By plotting numerous audiograms with age on the same graph, the age-related progression of hearing loss can be discerned.

### Other Terminology and Disclosure

When a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of the present disclosure.

All publications mentioned herein are incorporated herein by reference to disclose and describe the methods and/or materials for the purpose for which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible. This disclosure is intended to provide support for all such combinations.

As used herein, “may,” “may comprise,” “may be,” “can,” “can comprise” and “can be” all indicate something envisaged by the inventors that is functional and available as part of the subject matter provided.

### EXAMPLES

Various aspects and advantages of the disclosure are illustrated by the following non-limiting examples.

### Example 1

### Production of rAAV Expressing NF2 Isoform I

rAAV were produced using transgenes expressing a reporter such as green fluorescent protein (GFP) (SEQ ID NO: 14), or expressing a human NF2 cDNA (NF2 Isoform I cDNA of SEQ ID NO: 1) for use in a gene therapy re-expression approach for NF2 loss of function pathology.

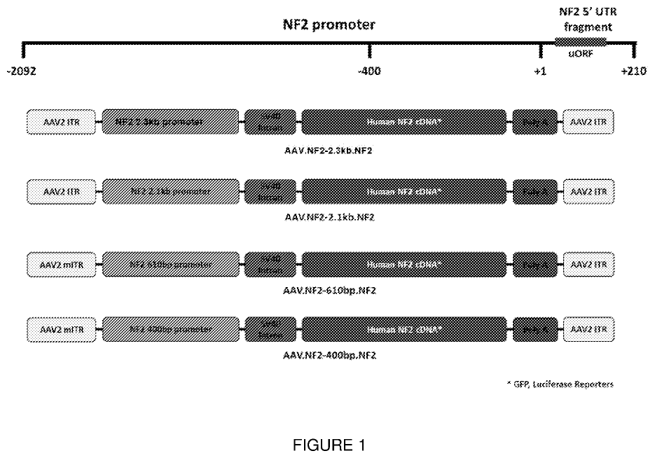

The transgenes included various NF2 promoters: 2.3 kb, 2.1 kb, 610 bp (also sometimes referred to herein as 609 bp) or 400 bp (FIG. 1) driving expression of the GFP or the Merlin protein. The transgene of SEQ ID NO: 6 includes the 400 bp promoter, the transgene of SEQ ID NO: 7 includes the 610 bp promoter, the transgene of SEQ ID NO: 8 includes the 2.1 kb promoter and the transgene of SEQ ID NO: 9 includes the 2.3 kb promoter. Promoters contemplated for use in methods herein are of a size that allow the transgene constructs to fit well in rAAV and also drive transgene expression to increase Merlin protein expression by at least 10% in transduced cells, focusing on expression in Schwann cells and meningeal cells which are the most frequently affected cell types in NF2.

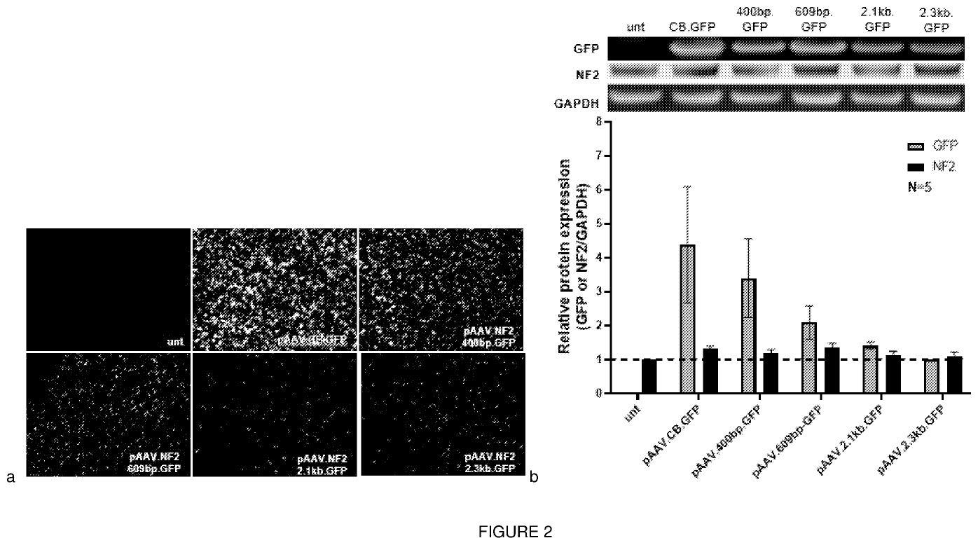

HEK293 cells were transiently transfected with pAAV.NF2.GFP reporter plasmids and analysed 72 hrs post-transfection for GFP expression. FIG. 2, (a) Fluorescence imaging of the transfected cells showed NF2 promoter-dependent differential expression of GFP based on the at different strengths relative to control chicken β actin (CB) promoter driving GFP. (b) Western blot analysis of transfected HEK293 cells lysates confirms the NF2 promoter-dependent expression of GFP in transfected cells with no effect on the endogenous NF2 protein levels between untransfected and transfected cells.

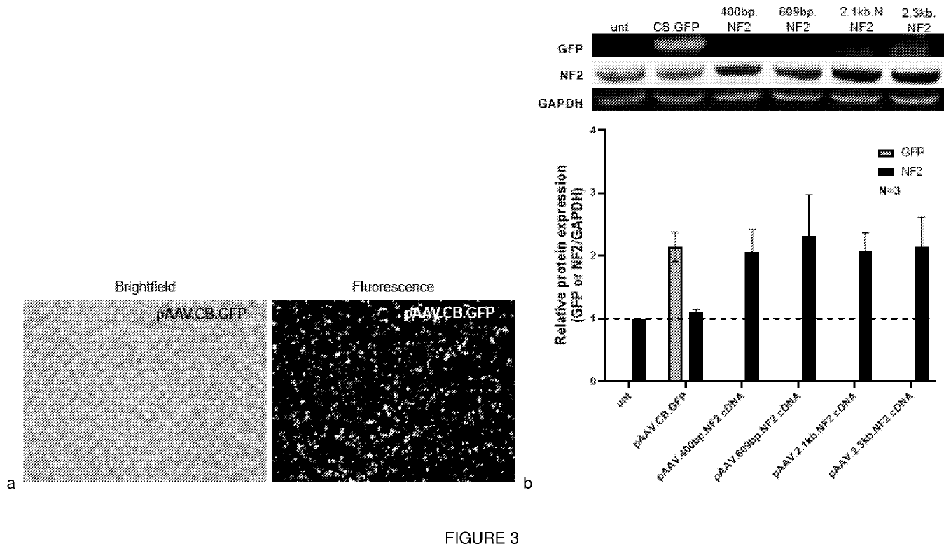

HEK293 cells were transiently transfected with pAAV.NF2.NF2 CDS plasmids and analyzed 72hrs post-transfection for NF2 expression. FIG. 3, (a) pAAV.CB.GFP plasmid treated HEK293 cells used as a transfection control showed robust GFP fluorescence confirming transfection efficiency, (b) Western blot analysis of transfected HEK293 cells confirms NF2 promoter-dependent differential overexpression of NF2 compared to pAAV.CB.GFP transfected and untransfected cells.

The transgenes were subcloned into AAV9 production plasmids, and scAAV and ssAAV were produced as described in Foust et al., Nat Biotechnol., 27(1): 59-65 (2009) by transient triple transfection of 293 cells using the double-stranded AAV2-ITR-based production plasmids, a plasmid encoding Rep2Cap9 sequence and an adenoviral helper plasmid pHelper. Merlin protein-encoding scAAV were named scAAV9.NF2-2.3kb.NF2, SCAAV9.NF2-2.1kb.NF2, scAAV9.NF2-610bp.NF2 (also sometimes referred to herein as scAAV9.NF2-609bp.NF2), and scAAV9.NF2-400bp.NF2. Merlin protein-encoding ssAAV were named ssAAV9.NF2-2.3kb.NF2, ssAAV9.NF2-2.1kb.NF2, ssAAV9.NF2-610bp.NF2 (also sometimes referred to herein as ssAAV9.NF2-609bp.NF2 because the promoter is actually 609 bp), and ssAAV9.NF2-400bp.NF2.

To test the different NF2 promoters, mice were injected with scAAV driving expression of GFP from the 400 bp or 610 bp NF2 promoter. Immunofluorescence showed that both promoters express GFP at varying levels in NF2 positive cells in the brain (mainly in the cerebral cortex, hippocampus, basal forebrain, vestibulocerebellum and vestibular nuclei), Schwann cells and sciatic nerve, but injection of the scAAV with the 400 bp promoter resulted in higher and more widespread GFP expression than the scAAV with the 610 bp promoter. Similarly, mice are injected with ssAAV driving expression of GFP from the 2.3 kb or 2.1 kb NF2 promoter, and expression is evaluated by immunofluorescence.

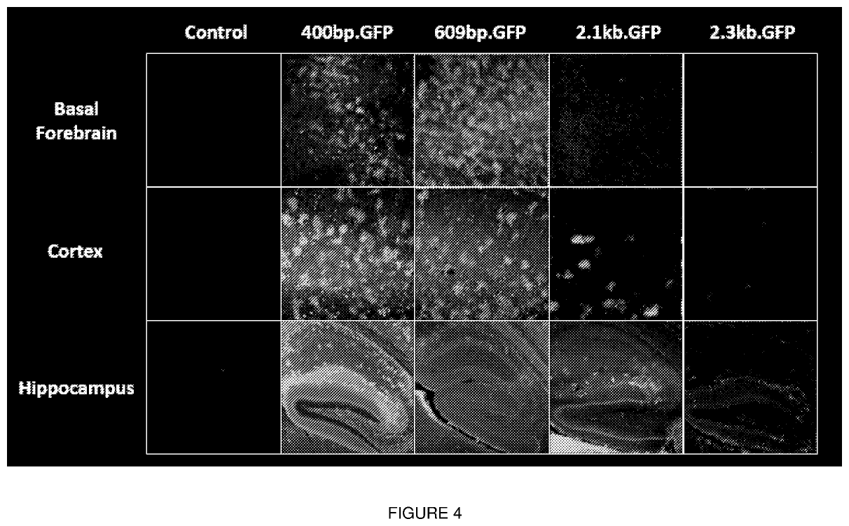

In another experiment to test the different promoters, scAAV9.NF2-400bp.GFP, scAAV9.NF2-609bp.GFP, ssAAV9.NF2-2.1kb GFP and ssAAV9.NF2-2.3kb GFP vectors were administered in neonatal mice via Intracerebroventricular (ICV) route at the same dose (5.0×10¹⁰ vg/animal) and sacrificed 1 month post injection. Different brain regions (basal forebrain, cortex, and hippocampus) examined for GFP expression by immunohistochemical fluorescence analysis showed that 400bp.GFP vector has overall higher GFP expression and co-localization with S100β, followed by the 609bp.GFP, 2.1kb.GFP and 2.3kb GFP. (FIG. 4)

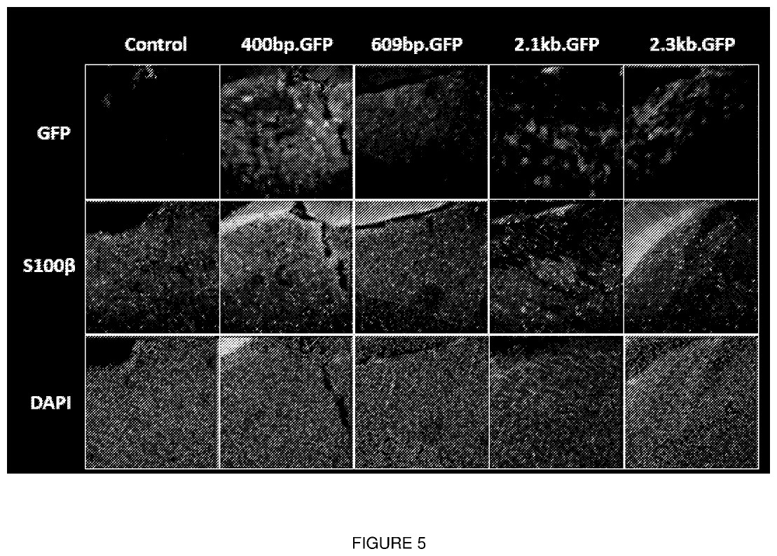

In yet another experiment, scAAV9.NF2-400bp.GFP, scAAV9.NF2-609bp.GFP, ssAAV9.NF2-2.1kb GFP and ssAAV9.NF2-2.3kb GFP vectors were administered in neonatal mice via the ICV route at the same dose (5.0×10¹⁰ vg/animal). The animals were sacrificed 1 month post injection and different brain regions were examined for GFP expression by immunohistochemical fluorescence analysis. In vestibular nuclei, 400bp.GFP vector showed overall higher GFP expression and co-localization with S100β, followed by 2.3kb.GFP and 2.1kb. (FIG. 5)

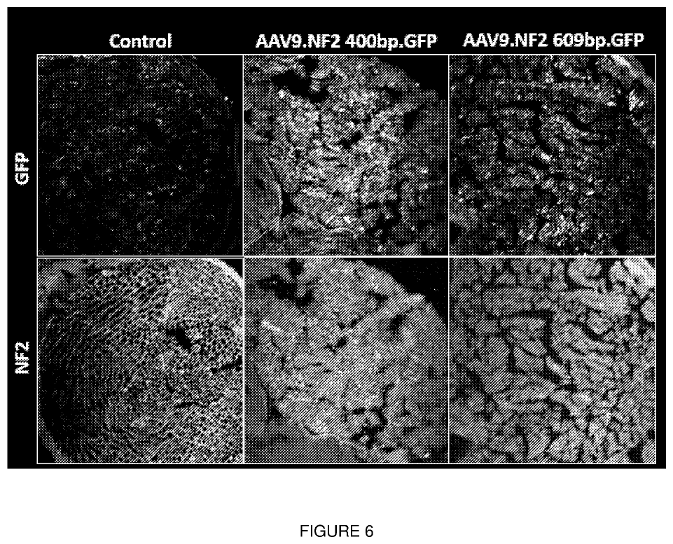

In another experiment, scAAV9.NF2-400bp.GFP and scAAV9.NF2-609bp.GFP vectors were administered in neonatal mice via the ICV route at the same dose (5.0×10¹⁰ vg/animal) and the animals were sacrificed 1 month post injection. Sciatic nerve of the mice were harvested and examined for GFP expression by immunohistochemical fluorescence analysis. 400bp.GFP vector showed significantly higher expression of GFP and colocalization with NF2 expressing cells in sciatic nerve as compared to 609bp.GFP vector. (FIG. 6)

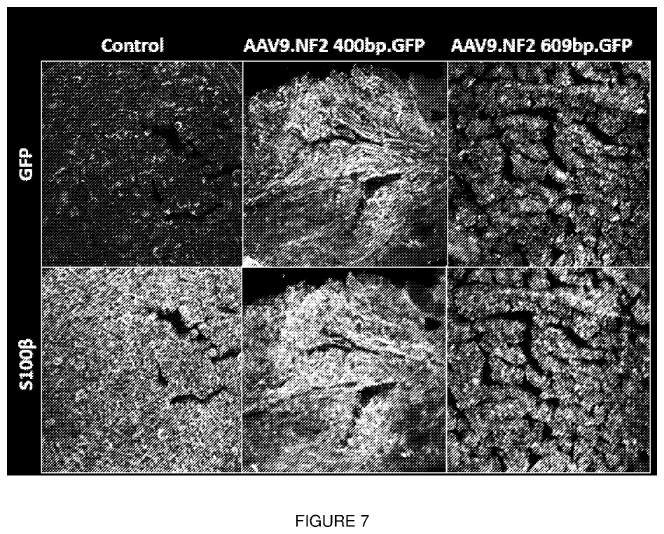

In another experiment, scAAV9.NF2-400bp.GFP and scAAV9.NF2-609bp.GFP vectors were administered in neonatal mice via ICV route at the same dose (5.0×10¹⁰ vg/animal) and the animals were sacrificed 1 month post injection. Sciatic nerve of the mice were harvested and examined for GFP expression by immunohistochemical fluorescence analysis. 400bp.GFP vector showed higher expression of GFP and colocalization with S100β expressing cells in sciatic nerve as compared to 609bp.GFP vector. (FIG. 7)

Corresponding scAAV with the chicken beta actin ubiquitous promoter or the Myelin Basic Protein promoter were also injected in mice. More expression of GFP was observed from the beta actin promoter in NF2 positive cells than from the Myelin Basic Protein promoter.

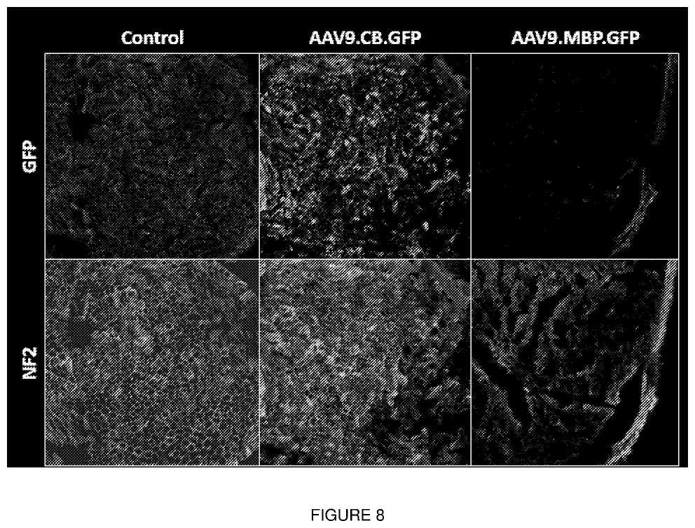

scAAV9.CB.GFP and scAAV9.MBP.GFP vectors were administered in neonatal mice via the ICV route at the same dose (5.0×10¹⁰ vg/animal) and the animals were sacrificed 1 month post injection. Sciatic nerve of the mice were harvested and examined for GFP expression by immunohistochemical fluorescence analysis. scAAV9.CB.GFP vector showed significant expression of GFP and colocalization with NF2 expressing cells in sciatic nerve as compared to MBP (myelin basic protein) driving GFP vector. (FIG. 8)

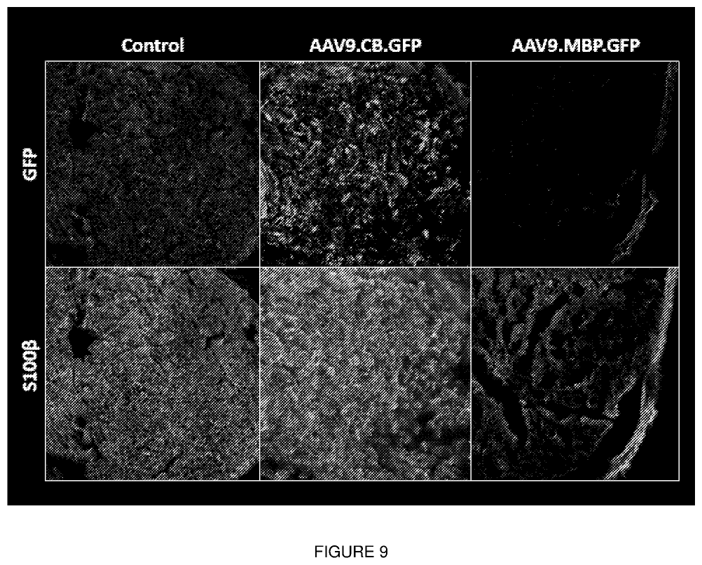

In another experiment, scAAV9.CB.GFP and scAAV9.MBP.GFP vectors were administered in neonatal mice via the ICV route at the same dose (5.0×10¹⁰ vg/animal) and the animals were sacrificed 1 month post injection. Sciatic nerve of the mice were harvested and examined for GFP expression by immunohistochemical fluorescence analysis. scAAV9.CB.GFP vector showed significant expression of GFP and colocalization with S100β expressing cells in sciatic nerve as compared to MBP (myelin basic protein) driving GFP vector. (FIG. 9)

Thus, co-localization of reporter GFP with S100β was observed in all regions (basal forebrain, cortex, hippocampus, vestibular nuclei) for 400bp, 609bp, and 2.1kB constructs. More co-localization of 400bp.GFP and 609bp.GFP was observed in basal forebrain, cortex, and hippocampus than 2.1kB.GFP. The 400bp.GFP vector co-localizes in the vestibular nuclei better than any other construct. The 400bp.GFP and 609bp.GFP constructs co-localize in sciatic nerves, with more co-localization for 400bp.GFP.

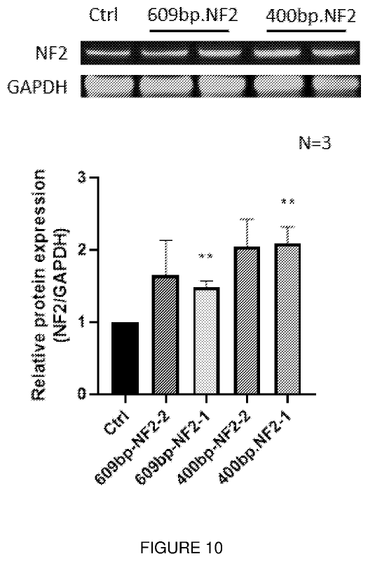

In another experiment, scAAV9.NF2-400bp.NF2 and scAAV9.NF2-609bp.NF2 vectors were administered in neonatal mice via the ICV route at the dose of 2.10E+10 vg and 3.50E+10 vg per animal, respectively. The animals were sacrificed 1 month post injection. Western blot analysis of whole brain lysates from the animals showed moderate overexpression of NF2 protein in both 400bp.NF2 and 609bp.NF2 treated mice relative to uninjected control. (FIG. 10)

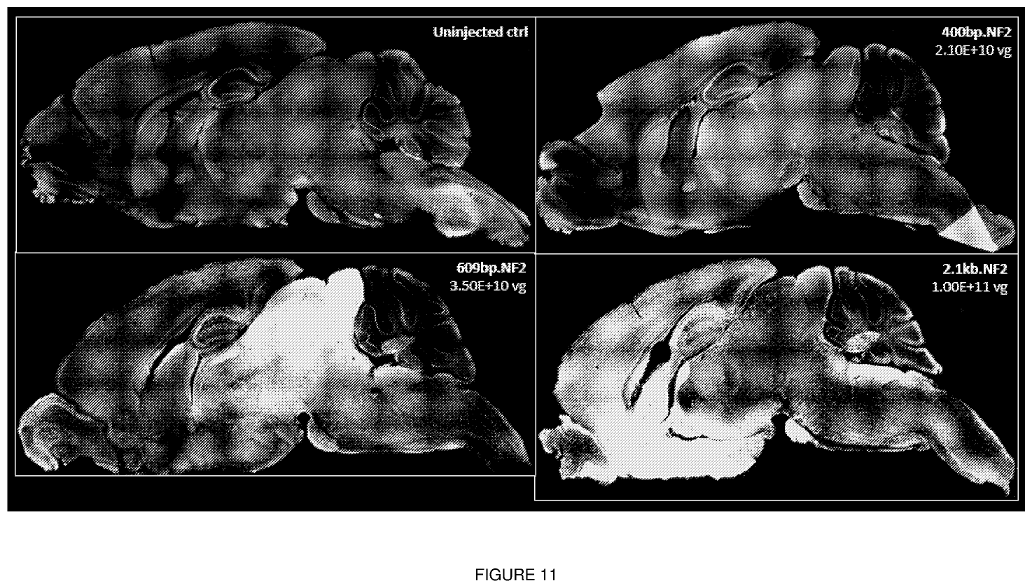

In another experiment, scAAV9.NF2-400bp.NF2, scAAV9.NF2-609bp.NF2 and ssAAV9.NF2-2.1kb.NF2 vectors were administered in neonatal mice via the ICV route at various doses and sacrificed 1 month post injection. Sagittal brain sections were examined for NF2 expression by immunohistochemical fluorescence analysis which demonstrated that all the treated mice showed NF2 overexpression. Especially, scAAV9.NF2 400bp.NF2 vector resulted in widespread NF2 overexpression when administered at even lower dose as compared to 609bp.NF2 and 2.1kb.NF2 vectors. (FIG. 11)

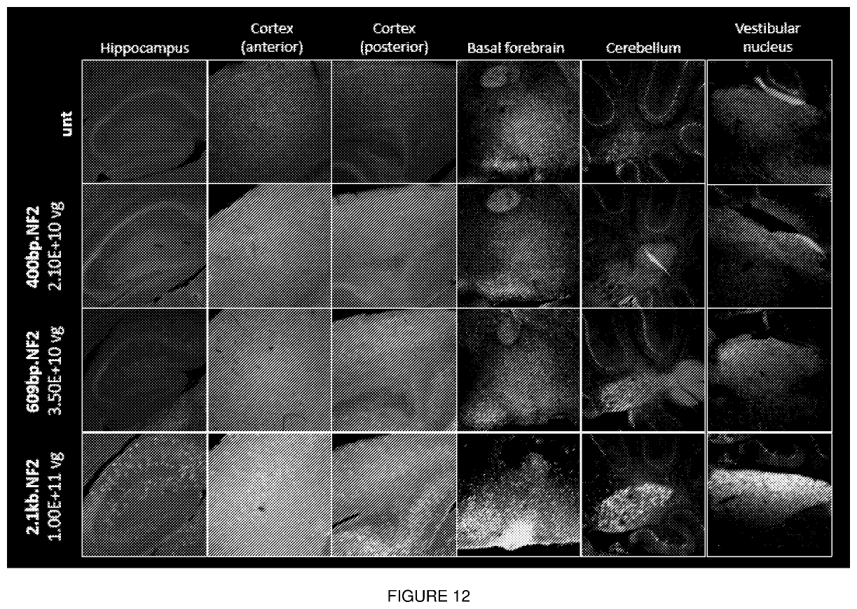

In another experiment, scAAV9.NF2-400bp.NF2, scAAV9.NF2-609bp.NF2 and ssAAV9.NF2-2.1kb.NF2 vectors were administered in neonatal mice via the ICV route at various doses and sacrificed 1 month post injection. Different brain regions (Hippocampus, anterior and posterior cortex, Basal forebrain, cerebellum, vestibular nucleus) were examined for NF2 expression by immunohistochemical fluorescence analysis. Mice treated with all three vectors showed NF2 overexpression in all the regions with 400bp.NF2 vector resulting in robust NF2 overexpression when administered at even lower dose as compared to 609bp.NF2 and 2.1kb.NF2 vectors. (FIG. 12)

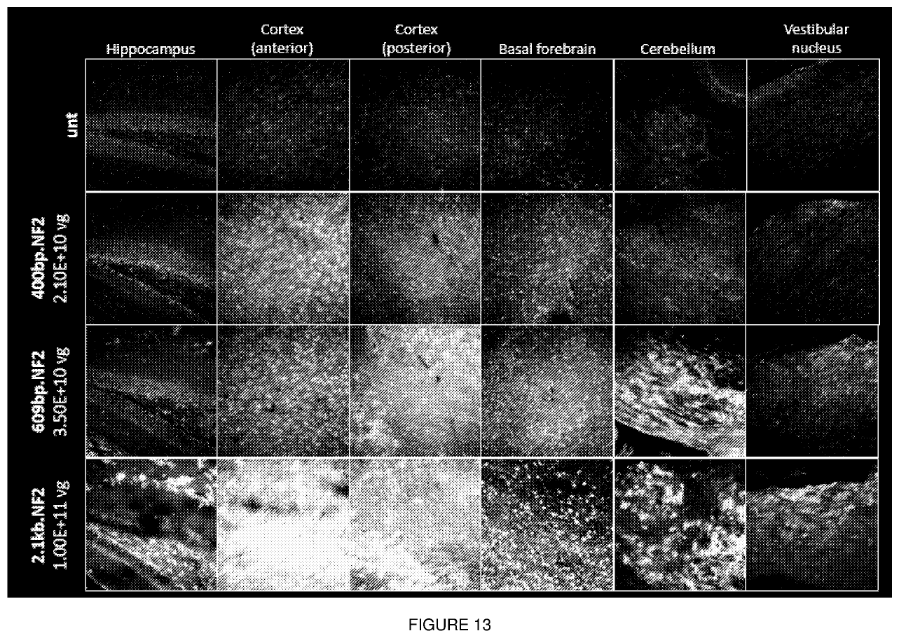

In another experiment, scAAV9.NF2-400bp.NF2, scAAV9.NF2-609bp.NF2 and ssAAV9.NF2-2.1kb.NF2 vectors were administered in neonatal mice via ICV route at various doses and sacrificed 1 month post injection. Different brain regions (Hippocampus, anterior and posterior cortex, Basal forebrain, cerebellum, vestibular nucleus) were examined for NF2 expression by immunofluorescence analysis. Mice treated with all three vectors showed NF2 overexpression in all the regions with 400bp.NF2 vector resulting in robust NF2 overexpression when administered at even lower dose as compared to 609bp.NF2 and 2.1kb.NF2 vectors. (FIG. 13)

The results demonstrate which promoter construct is most effective in targeting the NF2-relevant cell types and give an indication of potential expression levels of the transgene.

### Example 2

### Efficacy of AAV.NF2 in Postn-Cre; Nf2^{flox/flox} Mice

rAAV AAV.NF2 produced according to Example 1 are used in model Postn-Cre; Nf2^{flox/flox} mice which develop multiple spinal and cranial nerve tumors histologically identical to human schwannomas with 100% penetrance (Clapp et al., Hum Mol Genet 2015, 24: 1-8). The mice also exhibit functional impairments in hearing and balance due to the development of cranial nerve VIII tumors.

The Postn-Cre; Nf2^{flox/flox} mice are grown for six months to allow tumors to develop. The Postn-Cre; Nf2^{flox/flox} mice are treated either before or after tumor development with the AAV.NF2 by intravenous or intra-spinal fluid injection. Mice receive one intrathecal (IT) injection of about 1×10¹¹ vg to about 5×10¹¹ vg of rAAV AAV.NF2 [formulated, as one example, in 1×PBS and 0.001% Pluronic F68 (denoted as PBS/F68)]. Injection with an AAV-GFP vector is used as a control. At 8 months of age for pre-symptomatically treated mice or three months post injection for mice treated after tumor formation started, to assess hearing impairment in these mice, auditory brainstem response measurement is performed and compared to a separate cohort of mice. Then dorsal root ganglia and tumors from AAV.NF2 and AAV.GFP-treated mice are dissected to compare the treatment effect.

The experiment demonstrates re-expression of NF2 can prevent schwannoma development and hearing loss in this genetically engineered mouse model.

### Example 3

### Mouse In Vivo NF2 Model Demonstrating Gene Therapy in the Context of Various Human NF2 Mutations

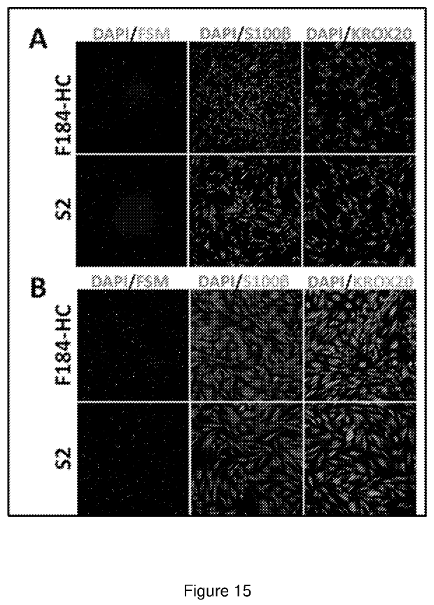

Gene replacement therapy is demonstrated in a human patient in vitro model using patient fibroblast cells reprogrammed into induced Schwann cells (iSC). The reprogramming method utilizes a chemically defined media to induce Schwann cell marker (S100 and Krox20) expression within 9 days. In addition, the reprogrammed cells downregulate fibroblast surface marker (FIG. 15A). The cells can be further grown and still maintain S100 and Krox20 expression (FIG. 15B) which allows for cellular expansion and use in a xenograft models.

The iSC cells are used in a patient-specific xenograft mouse model to evaluate the effects of the gene therapy approach in vivo. 5000 human patient iSC cells are injected directly into the mice sciatic nerves as previously described in Fuse et al., Mol Cancer Ther 2017, 16: 2387-2398. Starting two weeks and up to two months post injection the mice are sacrificed and tumor formation evaluated. Alternatively, instead of patient-derived iSC cells, patient-derived immortalized tumor cells (such as sporadic schwannoma cells lines or NF2 patient schwannoma and meningioma cells lines) are injected.

Aside from induced iSCs, patient-derived immortalized human tumor cells are also used for the xenograft model. Here, immortalized sporadic schwannoma cells as well as NF2 patient schwannoma and meningioma cells are injected into mice and further evaluated for tumor formation.

The mutations in the human patient fibroblast NF2 genes are contemplated to generate tumor formation in vivo in the mice. In addition, to further reduce NF2 gene expression to facilitate tumor growth, a lentiviral NF2 shRNA is used to reduce NF2 gene expression prior to Schwann cell differentiation to mimic the “second hit” the tumor forming cells experience in human patients. The ability of the Schwann cells generated from these NF2 shRNA expressing fibroblasts to produce tumors in vivo in the mice is compared with the ability of nontransduced fibroblasts.

The resulting mice that carry the different patient NF2 gene mutations are used to demonstrate rAAV AAV.NF2 effectiveness in preventing tumor formation or reducing tumor growth in the context of the patient mutations. Mice are treated with AAV.NF2 either twenty-four hours or two weeks following the iSC/immortalized human tumor cell injections into the sciatic nerve and/or tumors of mice. Mice receive one intrathecal (IT) injection or intratumoral injection in the range of about 1×10¹¹ vg to about 5×10¹¹ vg rAAV AAV.NF2 [formulated, as one example, in 1×PBS and 0.001% Pluronic F68 (denoted as PBS/F68)]. Mice are sacrificed, and number of tumors and size is assessed.

### Induced iSCs

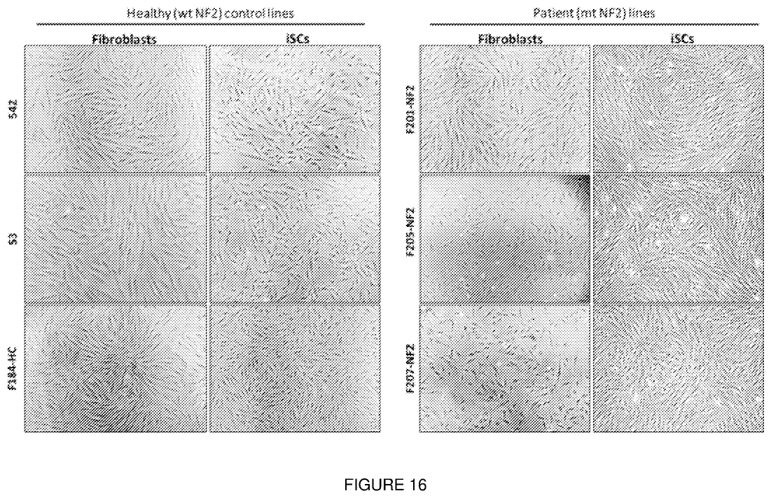

Three healthy control (wild-type) fibroblast lines and three patient (NF2-mutant) fibroblast lines were successfully converted into induced Schwann cells (iSCs) using serial treatments with six small molecules. Brightfield images of fibroblasts and respective iSCs showed differences in cell morphology. (FIG. 16)

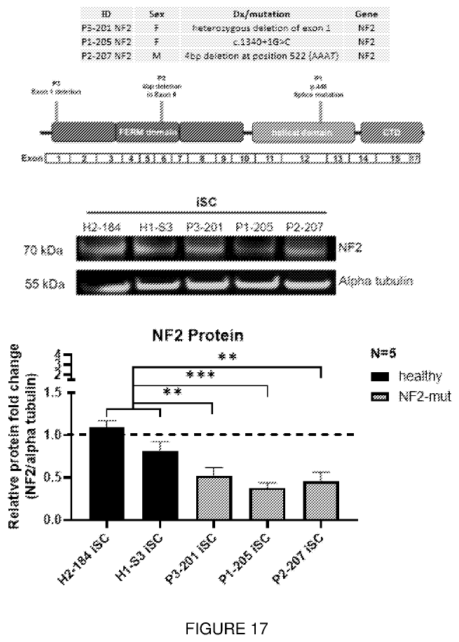

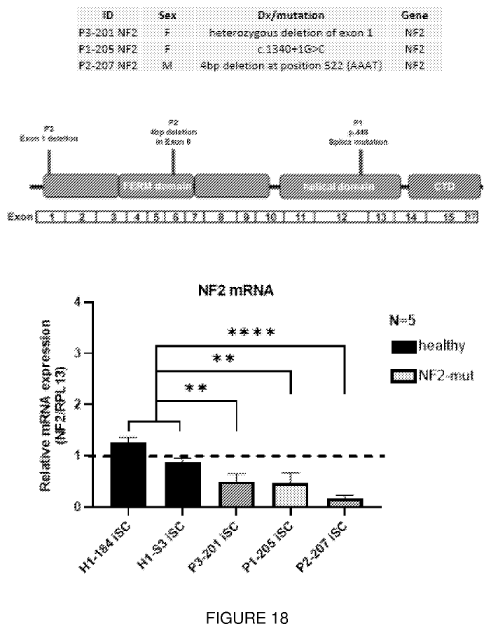

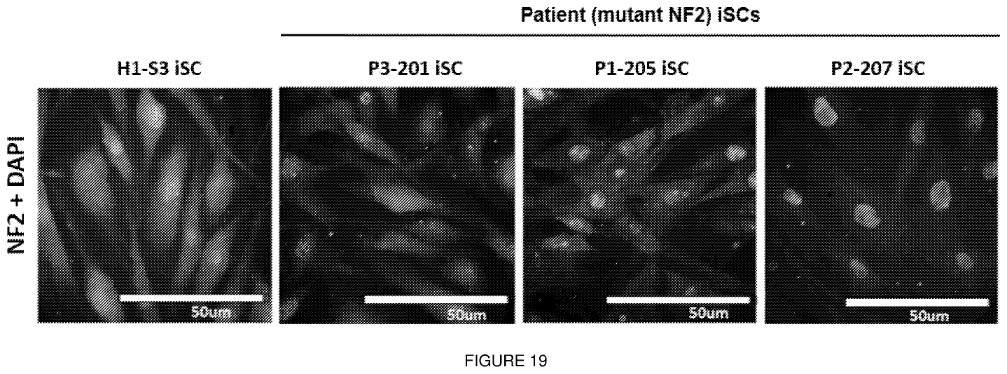

Western blot analysis of patient derived iSCs showed significantly reduced expression of NF2 protein as compared to the healthy iSCs. (FIG. 17) NF2 mRNA expression analysis by qRT-PCR of patient-derived iSCs also showed significantly reduced expression of NF2 protein as compared to the healthy iSCs. (FIG. 18) Immunofluorescence analysis of patient-derived iSCs showed reduced expression of total NF2 protein staining as compared to the healthy iSCs. (FIG. 19)

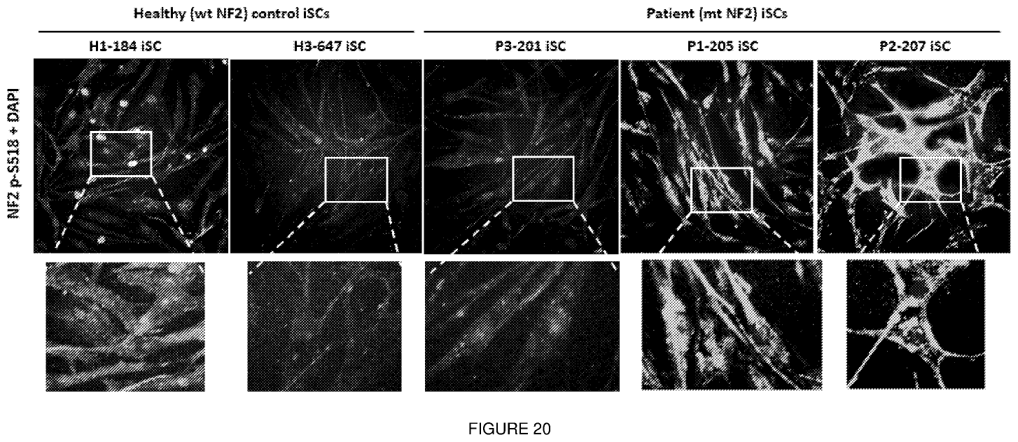

Immunofluorescence analysis of patient-derived iSCs showed increased expression of phospho-S518 merlin (inactive tumor suppressor) as compared to healthy control iSCs. The increase in phospho-S518 merlin expression seems to correlate with disease phenotype severity (patient 207 associated with more severe phenotype). In addition, phospho-S518 merlin is mislocalized in patient derived iSCs relative to healthy (wild-type NF2) control iSCs. (FIG. 20)

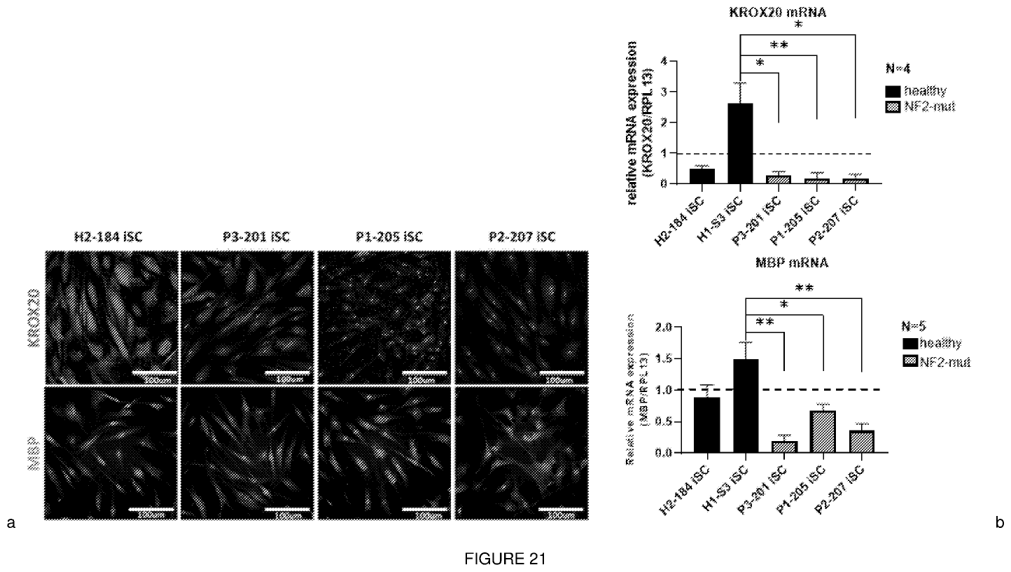

FIG. 21 shows reduced Schwann cell maturation marker expression in NF2 patient iSCs. a) Immunofluorescence analysis showed significantly reduced expression of mature myelinating Schwann cell markers (KROX20 and MBP) in NF2 patient derived iSCs as compared to healthy (wild-type NF2) control iSCs. b) mRNA expression analysis by qRT-PCR showed significantly reduced expression of KROX20 and MBP mRNA in NF2 patience iSCs compared to healthy control iSCs.

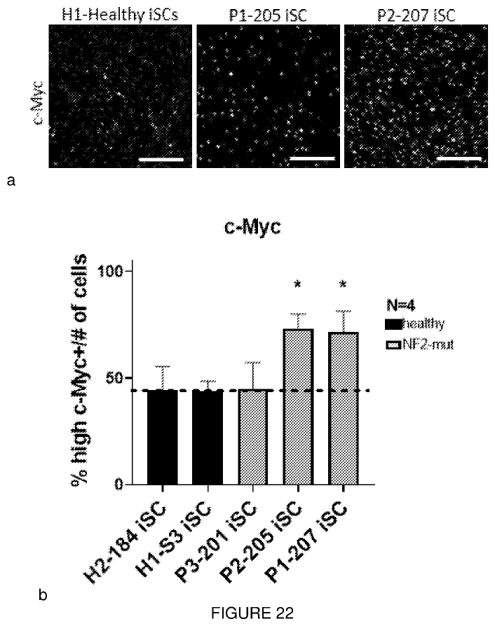

FIG. 22 shows increased stem cell marker expression in NF2 patient iSCs. a) Immunofluorescence analysis showed increased expression of stem cell marker c-Myc in 2 out of 3 NF2 patient derived iSCs as compared to healthy (wild-type NF2) control iSCs. b) Image-J analysis of c-Myc positive cells over total number of cells measured by DAPI showed significantly increased percentage of high c-Myc positivity in corresponding patience derived iSCs. Thus, it is contemplated c-Myc could be a potential biomarker for evaluating therapeutic strategies in vitro.

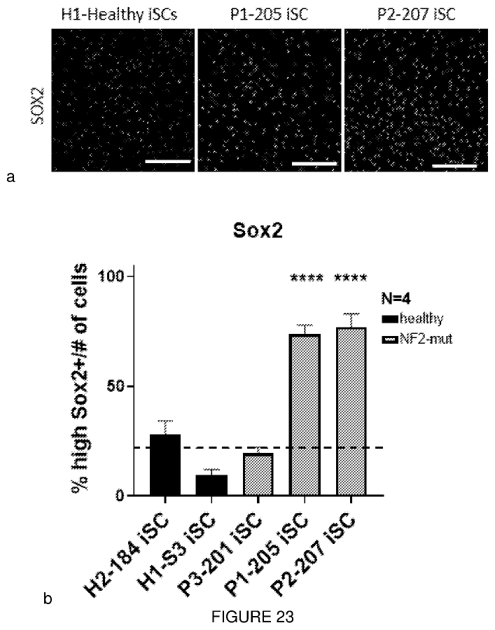

FIG. 23 shows increased stem cell marker expression in NF2 patient iSCs. a) Immunofluorescence analysis show increased expression of stem cell marker SOX2 in 2 out of 3 NF2 patient derived iSCs as compared to healthy (wild-type NF2) control iSCs. b) Image-J analysis of SOX2 positive cells over total # of cells measured by DAPI showed significantly increased percentage of high SOX2 positivity in corresponding patience derived iSCs. Thus, it is contemplated SOX2 could be a potential biomarker for evaluating therapeutic strategies in vitro.

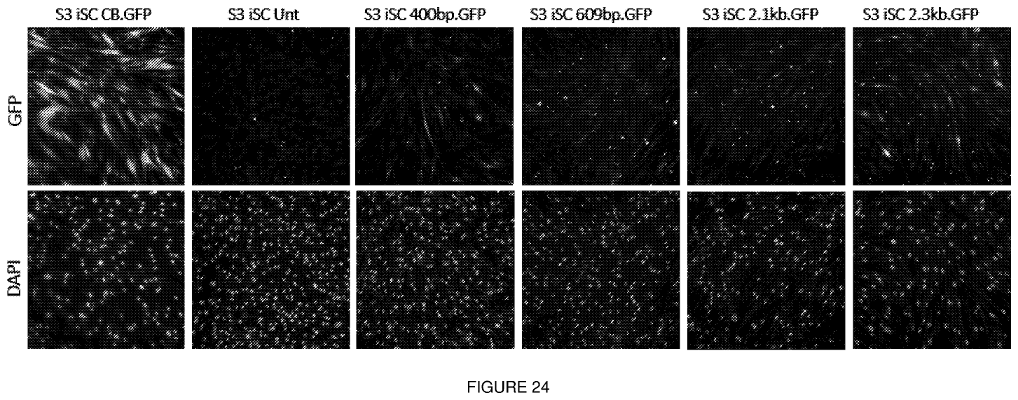

Healthy control iSCs were treated with Neuraminidase (NA) and transduced with the AAV9.NF2.GFP vectors (MOI 300K) and analyzed for GFP fluorescence 72 hrs post transduction. All four vectors showed GFP expression at varying intensities with AAV9.CB.GFP vector treated cells with high overexpression compared to the untreated control iSCs. (FIG. 24)

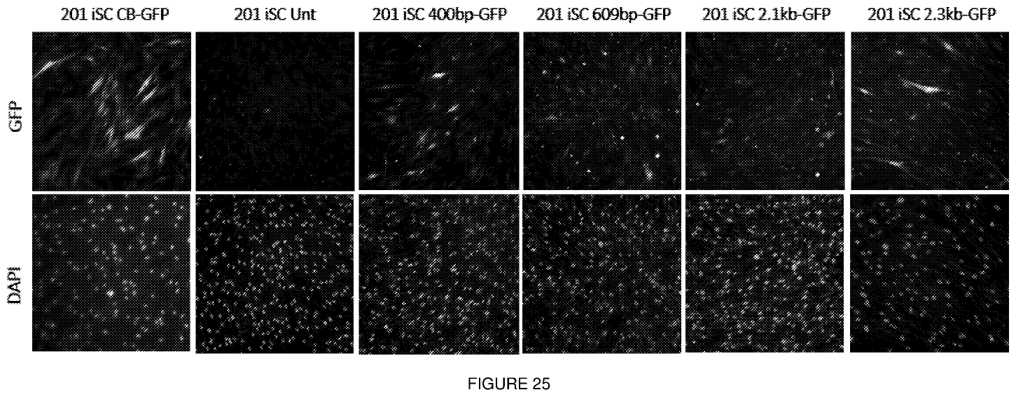

NF2 patient-derived iSCs were treated with Neuraminidase (NA) and transduced with the AAV9.NF2.GFP vectors (MOI 300K) and analyzed for GFP fluorescence 72 hrs post transduction. All four vectors showed GFP expression at varying intensities compared to untransduced iSC control. AAV9.CB.GFP vector treated cells with GFP overexpression serve as transduction control. (FIG. 25)

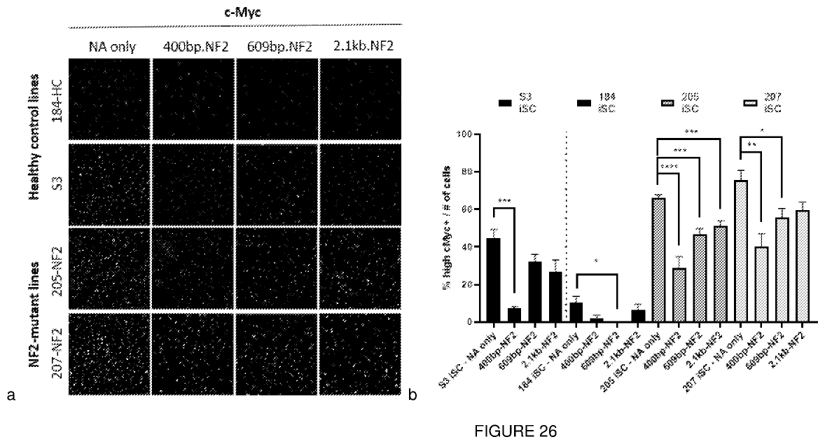

Healthy control- and NF2 patient-derived iSCs were treated with Neuraminidase (NA) and transduced (25K seeded, 72 hrs transduction) with scAAV9.NF2-400bp.NF2, scAAV9.NF2-609bp.NF2 or ssAAV9.NF2-2.1kb.NF2 (MOI 300K) and analyzed for c-Myc expression 72 hrs post-transduction. FIG. 26, a) Immunofluorescence analysis showed all three vectors reduced expression of stem cell marker c-Myc in NF2 patient derived iSCs as well as in healthy (wild-type NF2) control iSCs, b) Image-J analysis of high c-Myc positive cells over total number of cells measured by DAPI showed scAAV9. NF2-400bp.NF2 treatment resulted in highest reduction of high c-Myc positive cells as compared to other two vectors.

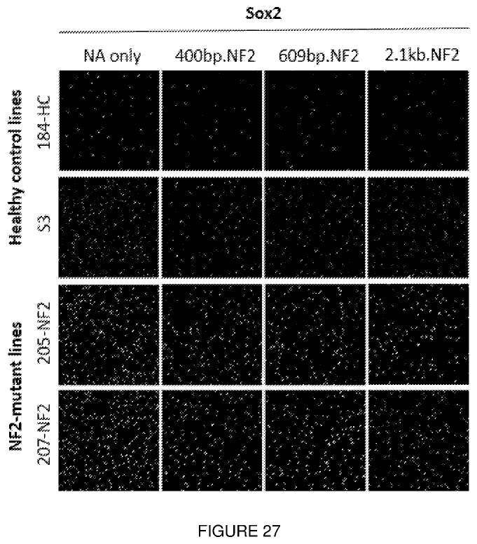

Healthy control- and NF2 patient-derived iSCs were treated with Neuraminidase (NA) and transduced with scAAV9.NF2 400bp.NF2, scAAV9.NF2 609bp.NF2 or ssAAV9.NF2 2.1kb.NF2 (MOI 300K) and analyzed for SOX2 expression 72 hrs post-transduction (n=2). Immunofluorescence analysis showed all three vectors reduced expression of stem cell marker SOX2 in NF2 patient derived iSCs as well as in healthy (wild-type NF2) control iSCs. (FIG. 27)

Thus, all patient iSCs have reduced NF2 levels when normalized to alpha-tubulin. Patient iSCs seem to have decreased SC maturation capacity. Patient iSC lines 205 and 207 show elevated c-Myc and Sox2 levels. AAV9.400bp.NF2 and AAV9.609bp.NF2 transduction can reduce c-MYC levels, with highest effect observed in the AAV9.400bp.NF2 vector.

As mentioned above, to further reduce NF2 gene expression to facilitate tumor growth, a lentiviral NF2 shRNA can be used to reduce NF2 gene expression prior to Schwann cell differentiation to mimic the “second hit” the tumor forming cells experience in human patients. In addition, the efficacy of NF2 downregulation after Schwann cell differentiation was also tested by using the same lentiviral NF2 shRNA construct to transduce healthy control and NF2 derived-induced Schwann cells.

To reduce the NF2 expression, an shRNA (shNF2) targeting NF2 sequence 5′-GGATGAAGCTGAAATGGAATA-3′ (SEQ ID NO: 47) was designed and cloned under control of the H1 promoter into LV-H2B-RFP (Addgene 26001). The resulting vector expressed shNF2 from the H1 promoter as well as H2B-RFP from the hPGK promoter which results in nuclear RFP expression. iSCs were transduced with LV-shNF2 and LV-RFP (transduction control) (100 MOI) and expanded until there were enough cells to sort using RFP based FACS. After sorting, cells were reseeded, expanded and maintained for the subsequent analysis (Western blot, qPCR and MTT cell proliferation assay, antiretroviral drug treatment) of sorted transduced cells.

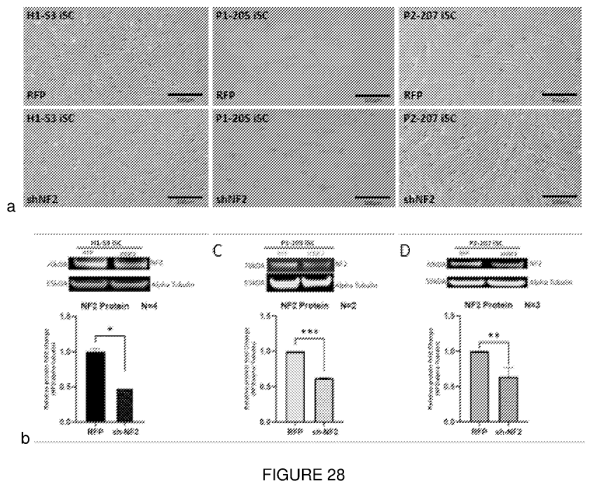

Sorted iSCs directly hit with shNF2 showed reduced NF2 levels. FIG. 28, (A) Fluorescence imaging showed sorted iSCs expressing RFP and shNF2 (black). (B, C, D) Western blot data showed NF2 protein levels are reduced by 40-50% in sorted healthy and patient iSCs transduced with shNF2 compared to RFP controls. T-test: *P≤0.05, **P≤0.01, ***P≤0.00.

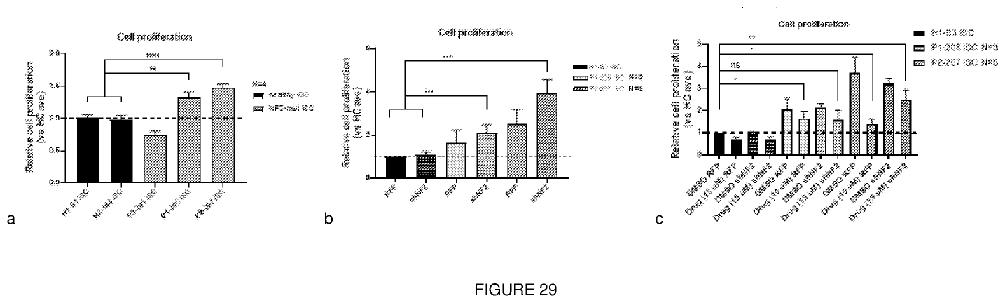

Cell proliferation (MTT) assay analyzed 48 hrs after seeding showed high proliferation rates in single-allele mutant NF2 patient iSCs and NF2 double knockdown patient iSCs. FIG. 29, (A) Two of three single-allele mutant NF2 iSCs proliferate 1.5-fold faster than healthy iSCs. (B) shNF2 knockdown patient iSCs proliferate 2- to 3-fold faster than healthy shNF2 knockdown iSCs. (C) RFP patient iSCs (single-allele mutant) and shNF2 (NF2 double knockdown) proliferation is reduced to closer to healthy levels after antiretroviral drug treatment. T-test: *P≤0.05, **P≤0.01; ***P≤0.001; ****P≤0.0001.

Thus, the shRNA based NF2 reduction in induced Schwann cells mimics the “second hit” the tumor forming cell experience in patients. This further NF2 reduction to mimic a second hit is contemplated to be an appropriate tumor modelling approach in vitro as shown by proliferation assays. In addition to use for gene therapy constructs, it is comtemplated these cells can be for drug screening of antiretrovirals and other small molecule drugs for treatment options for NF2 patients.

### Patient-Derived Immortalized Human Tumor Cells

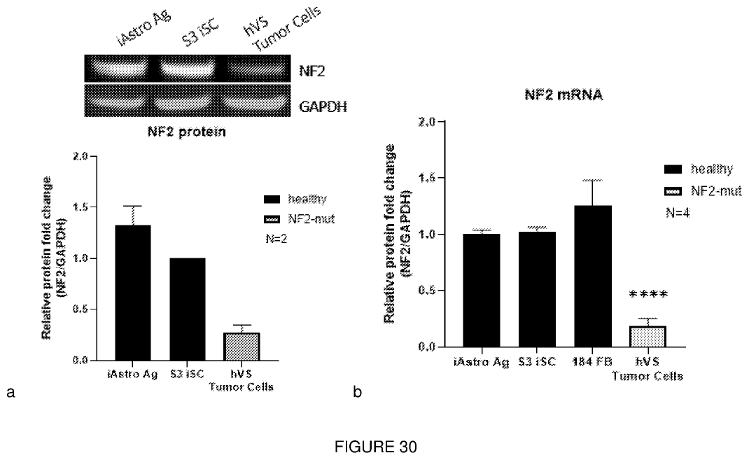

Other experiments showed reduced NF2 expression in hVS tumor cells. FIG. 30, a) Western blot analysis showed reduced expression of NF2 in hVS tumor cells as compared to healthy iAstrocytes and iSCs. b) NF2 mRNA expression analysis by qRT-PCR also showed corresponding reduced expression of NF2 in hVS tumor cells as compared to the healthy iAstrocytes, iSCs and fibroblasts.

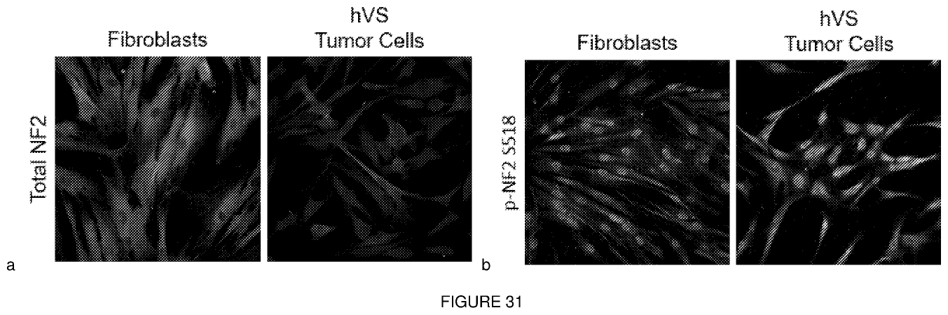

Immunofluorescence analysis of hVS tumor cells showed overall reduced expression of total NF2 (a) as compared to healthy fibroblasts. Importantly, when analyzed for the inactive form of the tumor suppressor (phosphorylated form), hVS tumor cells showed increased expression of phospho-S518 merlin (b) as compared to healthy control fibroblasts. (FIG. 31)

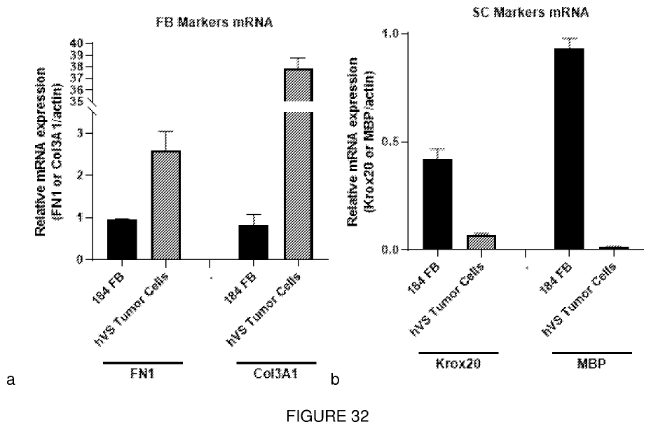

mRNA expression analysis by qRT-PCR showed higher expression of fibroblast markers (FN1 and Col3A) while reduced expression of mature schwann cells markers (Krox20 and MBP) in hVS tumor cells as compared to healthy control fibroblasts, suggesting the hVS tumor cells are farther away from mature and hence functional schwann cells. (FIG. 32)

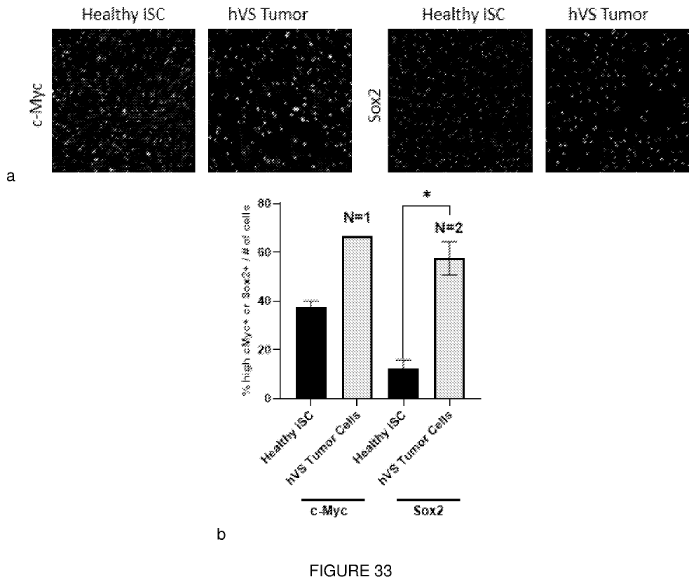

There was increased stem cell marker expression in hVS tumor cells. FIG. 33, a) Immunofluorescence analysis showed increased expression of stem cell markers c-Myc and SOX2 in hVS tumor cells as compared to healthy (wild-type NF2) control iSCs. b) Image-J analysis of c-Myc or SOX2 positive cells over total # of cells measured by DAPI showed higher percentage of high c-Myc/SOX2 positivity among hVS tumor cells as compared to healthy iSCs. Thus, c-Myc and SOX2 are contemplated to be biomarkers for evaluating therapeutic strategies in vitro.

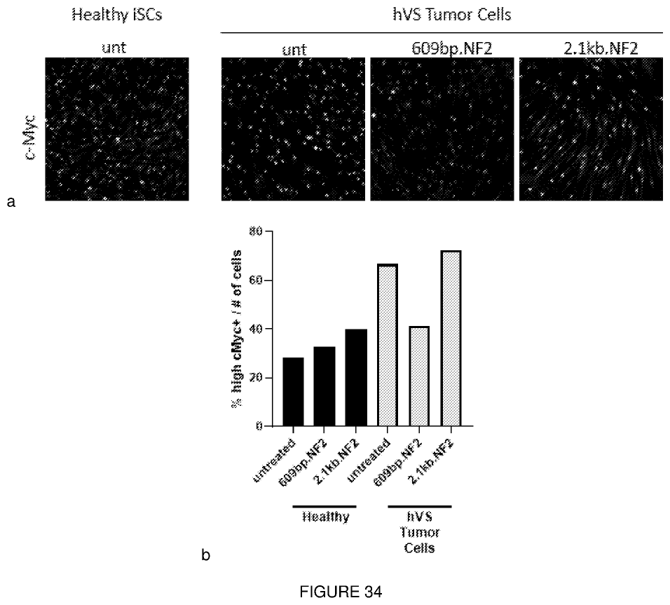

hVS tumor cells were treated with Neuraminidase (NA) and transduced with scAAV9.NF2 609bp.NF2 or ssAAV9.NF2 2.1kb.NF2 (MOI 300K) and analyzed for c-Myc expression 72 hrs post transduction. FIG. 34, a) Immunofluorescence analysis showed scAAV9.NF2 609bp.NF2 treatment reduced expression of stem cell marker c-Myc in hVS tumor cells. b) Image-J analysis of high c-Myc positive cells over total # of cells measured by DAPI showed scAAV9. NF2 609bp.NF2 treatment resulted in higher reduction of high c-Myc positive cells as compared to ssAAV9.NF2 2.1kb.NF2 treatment.

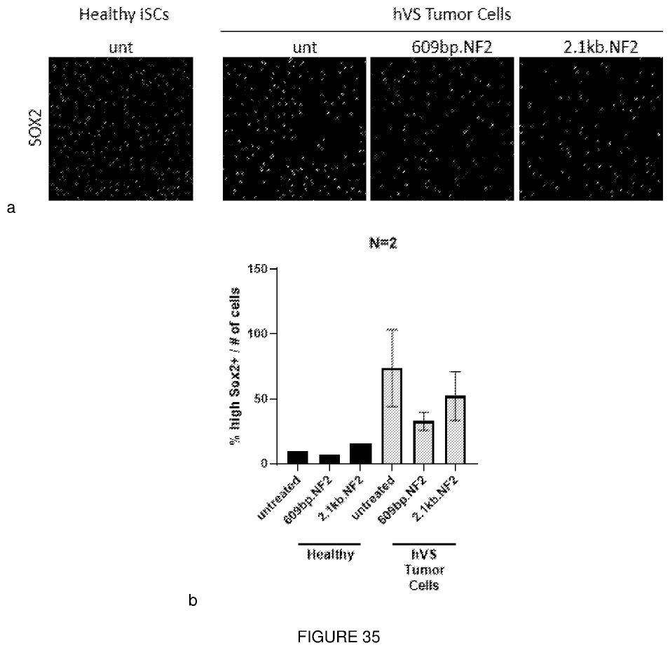

hVS tumor cells were treated with Neuraminidase (NA) and transduced with scAAV9.NF2 609bp.NF2 or ssAAV9.NF2 2.1kb.NF2 (MOI 300K) and analyzed for c-Myc expression 72 hrs post transduction. FIG. 35, a) Immunofluorescence analysis showed both vectors reduced expression of stem cell marker SOX2 in hVS tumor cells. b) Image-J analysis of high c-Myc positive cells over total number of cells measured by DAPI showed scAAV9. NF2 609bp.NF2 treatment resulted in higher reduction of high SOX2 positive cells as compared to ssAAV9.NF2 2.1kb.NF2 treatment.

Thus, hVS tumor cells have reduced NF2 levels. hVS tumor cells seem to have decreased SC maturation capacity. hVS tumor cells showed elevated c-Myc and Sox2 levels. AAV9.609bp.NF2 and AAV9.2.1kb.NF2 transduction can reduce c-MYC levels.

### SUMMARY

The above models demonstrate the effectiveness of gene therapy with the rAAV gene therapy vectors described herein for specific NF2 mutations while not requiring the generation of a new mouse model for each mutation. Furthermore, the models allow simultaneous evaluation of gene therapy and complementary therapeutic combination approaches in a highly variable patient population.

### Example 4

### Production of rAAV Expressing Phosphorylation-Resistant Merlin Protein

Phosphorylation of the Merlin protein can reduce its tumor suppressor activity by converting it to an open inactive conformation, inhibiting its binding other cellular proteins and the cytoskeleton, and/or leading to its degradation by ubiquitination. Phosphorylation-resistant Merlin proteins are therefore also provided herein for use as needed in gene therapy.

A Merlin protein with tumor suppressor activity can be resistant to post-translational negative regulation such as phosphorylation. Phosphorylation-resistant Merlin proteins with tumor suppressor activity provided herein have a phosphorylation site or sites removed by replacing one or more of the serine at position 10 of SEQ ID NO: 15, the threonine at position 230 of SEQ ID NO: 15, the serine at position 315 of SEQ ID NO: 15 and the serine at position 518 of SEQ ID NO: 15 with another amino acid such as alanine to prevent phosphorylation at/around that position. The amino acid replacement is effected, for example, by changing the polynucleotide sequence encoding the Merlin protein. For example, changing the thymidine at position 1609 of SEQ ID NO: 1 to a guanosine results in the substitution of an alanine residue for a serine residue in the encoded Merlin protein. SEQ ID NO: 16 set outs a polynucleotide with a guanosine at that position so that encodes a phosphorylation resistant Merlin protein. SEQ ID NOs: 17-20 set out transgenes including that polynucleotide.

HEK293 cells were transiently transfected with pAAV.NF2.NF2 S518A plasmids and analyzed 72hrs post-transfection for NF2 expression. (a) qRT-PCR analysis of HEK293 cells transfected with pAAV.NF2.NF2 S518A plasmids showed all four promoters driving NF2 S518A overexpression relative to pAAV.CB.GFP transfected and untransfected cells. (b) Western blot analysis of transfected HEK293 cells confirms NF2 promoter-dependent differential overexpression of NF2 S518A over pAAV9.CB.GFP transfected and untransfected cells. Historically, NF2 mRNA expression does not directly correlate to NF2 protein levels, suggesting additional post transcriptional regulation of NF2.

rAAV encoding a phosphorylation-resistant Merlin protein are produced as described in Example 1 using the transgenes set out in SEQ ID NOs: 17-20 each of which encode a S518A substitution (substitution of an alanine for a serine at position 518). As in Example 1, the four transgenes include different NF2 promoters: 2.3 kb, 2.1 kb, 610 bp (actually 609 bp) and 400bp (FIG. 1).

### Example 5

### In Vitro Targeting Efficiency of AAV9 Vectors for NF2 Re-Expression in Glioblastoma

Glioblastoma is a highly malignant tumor that originates from astrocytes. The U87 human glioblastoma cell line was derived from a stage three 44-year-old Caucasian female. Merlin has reduced expression in U87 high grade glioblastoma.

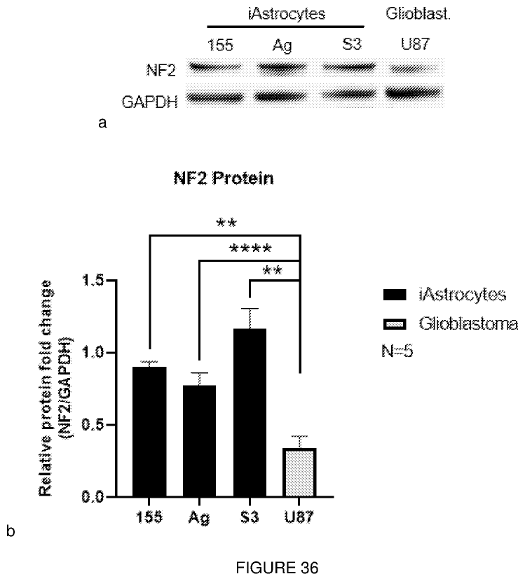

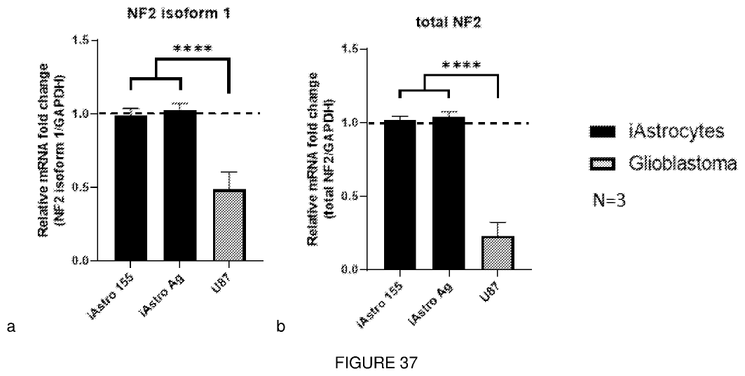

Western blot analysis of U87 glioblastoma cells showed lower NF2 protein expression compared to healthy control lines. (FIG. 36a-b) mRNA expression analysis by qRT-PCR showed lower expression of NF2 isoform 1 as well as total NF2 in U87 glioblastoma cells as compared to healthy control lines. (FIG. 37a-b)

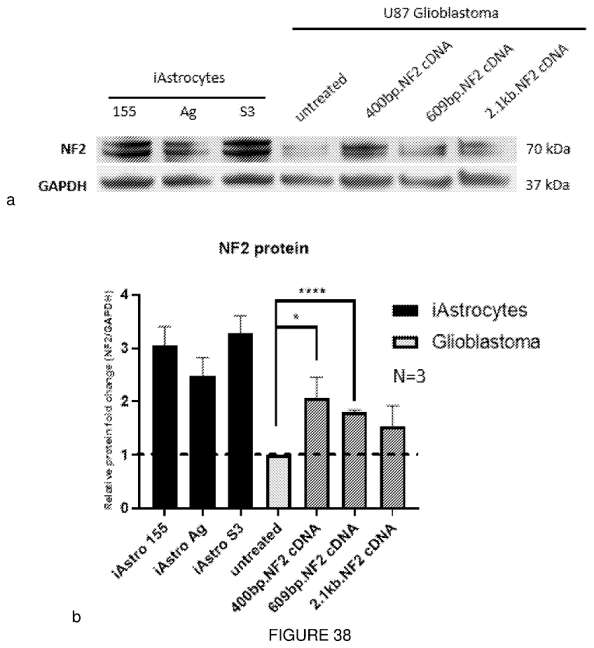

U87 glioblastoma cells were treated with Neuraminidase (NA) and transduced with SCAAV9.NF2-609bp.NF2 or ssAAV9.NF2-2.1kb.NF2 (MOI 300K) and analyzed for NF2 expression 72 hrs post transduction. FIG. 38, a) Western blot analysis showed all three vectors restore the NF2 expression in treated U87 Glioblastoma cells. b) Quantification of the western blot analysis reveals scAAV9. NF2-400bp.NF2 and scAAV9. NF2-609bp.NF2 treatments resulted in higher increase in NF2 expression in U87 glioblastoma cells as compared to ssAAV9.NF2-2.1kb.NF2 treatment.

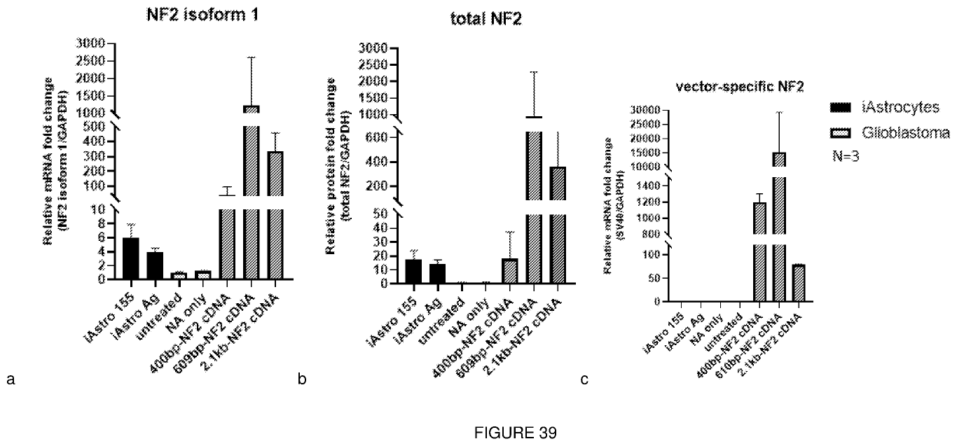

U87 glioblastoma cells were treated with Neuraminidase (NA) and transduced with scAAV9.NF2-609bp.NF2 or ssAAV9.NF2-2.1kb.NF2 (MOI 300K) and analyzed for NF2 expression 72 hrs post transduction. mRNA expression analysis was performed to determine the particular NF2 isoforms. FIG. 39, a-b) mRNA expression analysis using primers specific to NF2 isoform 1 as well as total NF2 showed corresponding increase in AAV9.NF2 treated U87 glioblastoma cells. c) Analysis of vector-derived NF2 transcript also showed significant increase in vector specific NF2 in treated U87 cells with higher expression of NF2 in U87 glioblastoma cells treated with scAAV9.NF2-400bp.NF2 and scAAV9.NF2-609bp.NF2 as compared to ssAAV9.NF2-2.1kb.NF2 treatment. However, high RNA expression with lower protein expression may suggest that the NF2 protein might be getting degraded.

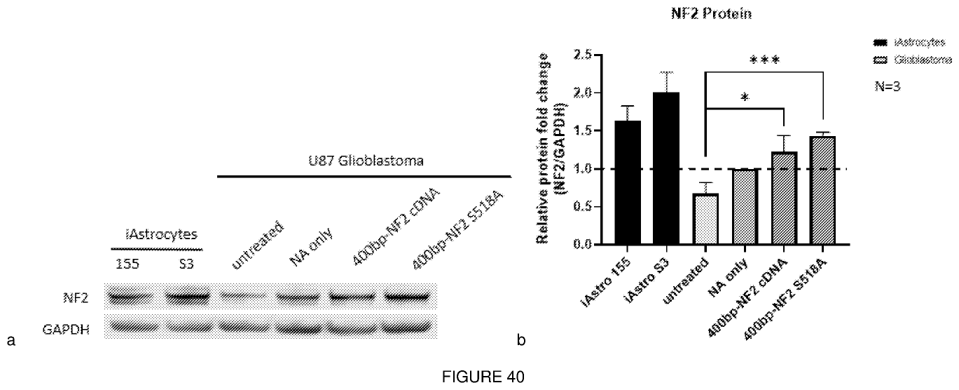

U87 glioblastoma cells were treated with Neuraminidase (NA) and transduced with scAAV9.NF2-400bp.NF2 (wild-type NF2 isoform 1) or scAAV9.NF2-400bp.NF2 S518A (Phospho-resistant NF2 isoform 1) (MOI 300K) and analyzed for NF2 expression 72hrs post transduction. FIG. 40, a) Western blot analysis showed both the vectors restore the NF2 expression in treated U87 Glioblastoma cells. b) Quantification of the western blot analysis reveals scAAV9. NF2-400bp.NF2 S51A treatment resulted in even higher increase in NF2 expression in U87 glioblastoma cells as compared to scAAV9.NF2-400bp.NF2 treatment.

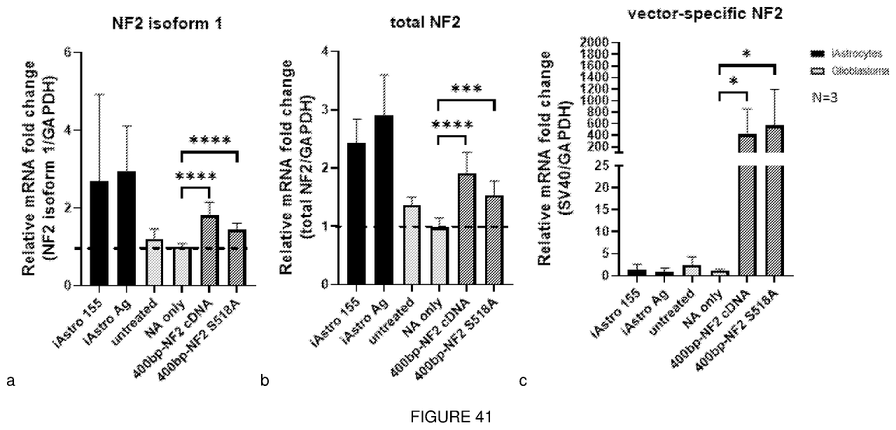

U87 glioblastoma cells were treated with Neuraminidase (NA) and transduced with scAAV9.NF2-400bp.NF2 (wild-type NF2 isoform 1) or scAAV9.NF2-400bp.NF2 S518A (Phospho-resistant NF2 isoform 1) (MOI 300K) and analyzed for NF2 expression 72 hrs post transduction. mRNA expression analysis was performed to determine the particular NF2 isoforms. mRNA expression analysis using primers specific to NF2 isoform 1 (a), total NF2 (b) and vector derived NF2 transcript (c) showed significantly higher expression in U87 glioblastoma cells treated with both scAAV9.NF2-400bp.NF2 and scAAV9.NF2-400bp.NF2 S518A Importantly, scAAV9.NF2-400bp. NF2 S518A treatment resulted high RNA expression as well as high protein expression in U87 Glioblastoma cells. (FIG. 41)

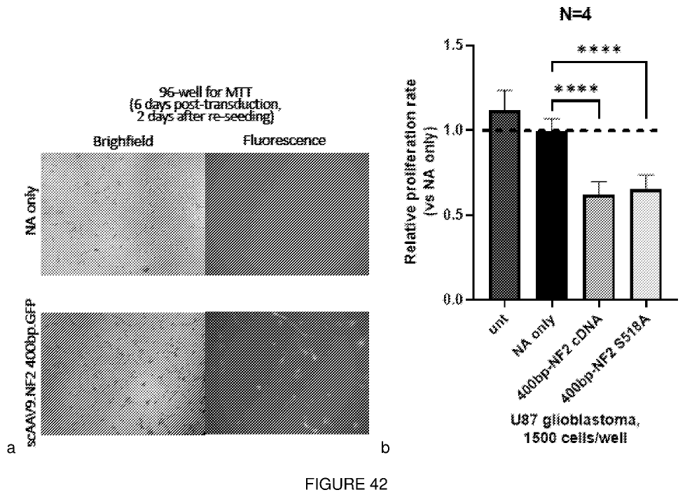

U87 glioblastoma cells were treated with Neuraminidase (NA) and transduced with scAAV9.NF2-400bp.NF2 (wild-type NF2 isoform 1), scAAV9.NF2-400bp.NF2 S518A (Phospho-resistant NF2 isoform 1) or scAAV9.NF2-400bp.GFP)transduction control only) (MOI 300K) and analyzed for cell proliferation by MTT assay 144 hrs post transduction. FIG. 42, a) brightfield and fluorescence images of control U87 glioblastoma cells after NA treatment and scAAV9.NF2-400bp.GFP showed cell density and efficient tAAV9 transduction as evident by GFP expression. b) U87 glioblastoma cells transduced with scAAV9.NF2-400bp.NF2 and scAAV9.NF2-400bp.NF2 S518A showed reduced proliferation rate as compared to NA only treated and untreated glioblastoma cells.

Thus, AAV9.400bp.NF2 and AAV9.609bp.NF2 were the most effective at increasing NF2 levels. AAV9.400bp.NF2 cDNA and AAV9.400bp.NF2 S518A transduction reduces glioblastoma proliferation rate. NF2 mRNA levels are really high, yet only small amount of NF2 detected, therefore it is also contemplated that phosphorylation resistant NF2 protein is useful in treatment methods.

### Various Materials and Methods Used in the Examples

AAV construct and vector production: Final constructs contain AAV2 inverted terminal repeats (ITR) flanking the various promoters (CMV enhancer/beta-actin (CB) promoter, Myelin Basic protein (MBP) promoter, and NF2 promoters), SV40 intronic sequence, and GFP, NF2 or NF2 S518A coding sequence followed by the BGH Poly A sequence. The four truncated NF2 promoter sequences were amplified by PCR from human genomic DNA using combinations of the primers listed in Table 1. The amplicons were used to replace the CB promoter by restriction enzyme digestion with Kpnl and Pstl flanking sequences to generate NF2 promoter driving GFP expression. To generate the constructs expressing wild-type NF2 cDNA, the full length wild-type NF2 isoform 1 sequence was amplified from a commercially available expression plasmid (Origene, SC124024) using primers listed in Table 1 below. The NF2 isoform 1 sequence was then used to replace GFP cDNA sequence in the four NF2.GFP constructs by restriction enzyme digestion with Agel and BsrGl flanking sequences. To generate the constructs expressing phosphorylation resistant NF2 S518A, the NF2 cDNA sequence containing the point mutation conferring the S518A substitution was commercially synthesized (GenScript, Piscataway, NJ), amplified, and used to replace the GFP cDNA sequence in the four NF2.GFP constructs by restriction enzyme digestion with Agel and BsrGl flanking sequences. The NF2 promoter-NF2 cDNA and NF2 promoter-GFP cDNA expression cassettes that were constructed were cloned in self-complementary and single stranded AAV plasmids in forward or reverse orientations with Ampicillin as well as Kanamycin resistance genes in either orientation. All vector DNA constructs were sequence-verified before being packaged into AAV9 capsid (self-commentary or single-stranded) by small-scale vector production (Andelyn Biosciences, Columbus, OH).

Animals: All procedures performed were in accordance with the National Institutes of Health guidelines and approved by the Research Institute at Nationwide Children's Hospital (Columbus, OH) Institutional Animal Care and Use Committees (IACUC). Experiments were performed on wild-type C57BI/6 mice (Strain #000664. The Jackson Laboratory, ME, USA). P0-P2 pups were used for unilateral intracerebroventricular (ICV) injections of AAV9 vectors. Daily evaluation was done on animals to check for health and sacrificed at 1 month post-injection for analysis.

Injections: For ICV injections of mice at P0-P2, the pups were anesthetized on ice for 10 minutes prior to injection. Injection was performed with Hamilton Syringe attached to a 33G needle as previously described. The AAV9 vectors were diluted in phosphate-buffered saline (PBS) for lower doses. The total volume injected for each animal was 5 μL.

Cell lines: Human skin fibroblasts were isolated from skin punch samples obtained from NF2 patients. Informed consent was obtained from all subjects before sample collection. Receipt of human samples was granted through Nationwide Children's Hospital Institutional Review Board. The heterozygous NF2 mutations present in each patient (P1-205, P2-207, and P3-201) were confirmed by amplicon sequencing using the Illumina HiSeq 2000. Established skin fibroblast cell lines were cultured and expanded in Dulbecco's Modified Eagle Medium (DMEM; Gibco, Waltham, MA) supplemented with GlutaMAX™, 10% fetal bovine serum (FBS) (Gibco) and 1% antibiotic-antimycotic (Gibco).

The immortalized human vestibular schwannoma (hVS) cell line was previously described [Chang et al, PLOS ONE, 16(7): e0252048. https://doi.org/10.1371/journal.pone.0252048 (2021)]. The hVS cells were cultured in Dulbecco's Modified Eagle Medium/Nutrient Mixture F-12 (DMEM/F-12) supplemented with GlutaMAX™, 10% FBS and 1% antibiotic-antimycotic, 100 μM recombinant human HRG1-beta 1 (HRG; R&D Systems, Minneapolis, MN), and 10 mM forskolin (Peprotech, Cranbury, NJ). Tissue-culture treated plates were coated with 50 μg/mL poly-L-lysine (70-150 kDa) (Sigma-Aldrich, St. Louis, MO) in and 0.1M boric acid and 0.01% borate for 30 min at RT, washed twice with 1×PBS, and coated with 4 μg/mL laminin mouse protein (Gibco) for 30 min at RT prior to seeding the hVS cells.

The U87 glioblastoma cell line (received from Dean Lee, Nationwide Children's Hospital, Columbus, OH) and HEK293 human embryonic kidney cells were cultured in Dulbecco's Modified Eagle Medium (DMEM) supplemented with 10% FBS and 1% antibiotic-antimycotic.

Direct conversion of fibroblasts to induced Schwann cells: Patient and healthy fibroblasts were directly converted into induced Schwann cells (ISCs) using a modified protocol previously described by Kitada et al. (2019). Briefly, fibroblasts between passage 5-9 were seeded at a density of 790 cells/cm² in tissue culture treated plates with DMEM supplemented with GlutaMAX™, 10% FBS, and 1% antibiotic-antimycotic. Twenty-four hours after seeding, media was changed to DMEM only without serum added and supplemented with 1 mM β-mercaptoethanol (BME; Sigma-Aldrich). The next day, media was changed with DMEM supplemented with 10% FBS and 35 ng/ml retinoic acid (Sigma-Aldrich) and were incubated in this medium for 72 hrs. Afterwards the cells were washed with 1× Dulbecco's phosphate buffered saline (DPBS) and incubated in fresh iSC induction medium (DMEM supplemented with GlutaMAX™, 5% FBS, 1% antibiotic-antimycotic, with the following small chemicals: 5 μM forskolin (PeproTech), 10 ng/ml human basic fibroblast growth factor (bFGF; Peprotech), 10 ng/ml platelet-derived growth factor-AA (PDGF-AA; Peprotech), and 200 ng/mL HRG). The cells were incubated in this medium, split at 90% confluence for expansion, and subsequently characterized through microscopy and molecular and cellular assays.

Transfection of HEK293 cells for evaluation of GFP and NF2 expression: HEK293 cells were seeded at 1.5×10⁶ cells/well in a 6-well plate (2×10⁵ cells/cm² cell density). Cells were transfected at 80% confluence with 1.5 μg of purified plasmid constructs by calcium chloride treatment. Briefly, plasmid DNA was added to 250 mM CaCl₂ and swirled gently before adding an equal volume of 2× HEPES-buffered saline (HBS) pH 7.05 to each tube. Two milliliters of transfection media (DMEM 2% FBS) was added to the plasmid solutions, mixed by pipetting, and added carefully to the cells. After 12 hrs, the transfection mixture was replaced with fresh media and incubated for 72-96 hrs to allow transient expression of GFP or NF2 from the transfected constructs. Cells were harvested by trypsinization and used for subsequent analysis by Western blot for qPCR for GFP and NF2 expression.

AAV Transduction of cells in culture: hVS tumor cells and iSCs were seeded in 24 well plates with glass coverslips at 25,000 cells/well for subsequent analysis by immunostaining. U87 glioblastoma cells were seeded in 12-well tissue culture plates at 80,000 cells/well for analysis by Western blot, qPCR, and MTT cell proliferation assay. All culture vessels were pre-treated with 5 μg/mL human fibronectin in DPBS for 30 min at RT to enhance cell adhesion. The next day, cells were carefully washed thrice with 2% FBS medium (hVS), 5% FBS medium (iSCs), or no serum medium (U87) and then incubated in 100 mU/mL neuraminidase (NA) (Sigma-Aldrich) for 2 h at 37° C./5% CO₂. NA was carefully removed and the cells washed again three times with their respective media described above, and treated with the AAV vectors diluted in media at 300,000 DRP/mL. The plates were incubated at 4° C. for 1 h before transferring to a humidified incubator at 37° C./5% CO₂. At 48 h post-transduction, fresh complete media was added to the cells and continued to incubate until 72-96 h for subsequent analysis of transduced cells.

Immunofluorescence staining: Fibroblasts, hVS tumor cells, and iSCs were seeded in 24 well plates with glass coverslips. The next day the cells were fixed with 4% paraformaldehyde for 12 min at RT, washed three times with DPBS, and blocked in DPBS with 10% goat serum, 0.1% Triton X-100, and 0.1% Tween-20 for 1 h at RT. Cells were then incubated in primary antibody overnight at 4° C. with gentle shaking. All primary antibodies were diluted in blocking solution. Respective dilutions and providers are listed in Table 2 below. The following day, cells were washed 3× in DPBS before incubating in Alexa Fluor-conjugated secondary antibodies (Invitrogen) and DAPI (Thermo Fisher Scientific) diluted in blocking solution for 1 h at room temperature. Following three washes with DPBS, the coverslips were mounted on slides with Vectashield (Vector Labs, Burlingame, CA) and sealed. Images were captured with Nikon Eclipse Ti2-E motorized inverted microscope and NIS-Elements imaging software. Quantitation of cells with high c-Myc or Sox2 staining was performed using the ImageJ/Fiji image processing and analysis software (Schneider et al., 2012)

Immunohistochemistry: Mouse brains and sciatic nerves were dissected from deeply-anesthetized mice that were trans-cardially perfused with 10 mL 0.9% saline. Brains were post-fixed in 4% PFA at 4° C. until sectioning, mounted on a vibratome stage using 5% agarose in water, and cut sagittally at 40 μm thickness. Sections were put sequentially into adjacent wells of a multi-well plate with 0.1 M PBS with 0.2% sodium azide for storage in 4° C. until immunostaining. The sections were then rinsed thrice in PBS with 0.1% Tween-20 (PBST), then blocked in DPBS with 10% normal donkey serum, 1% Triton X-100, and 0.02% sodium azide for 2 h at RT. Sections were incubated in primary antibodies diluted in blocking solution for 48 h at 4° C. with gentle shaking. Respective dilutions and providers for all antibodies used are listed in Table 2. Washing was done 3X with DPBS prior to incubation in Alexa Fluor-conjugated secondary antibodies and DAPI diluted in blocking solution for 2 h at room temperature with gentle shaking. The sections were washed again in PBS 3× then mounted on slides with polyvinyl alcohol mounting medium with DABCO antifading (PVA-DABCO; Sigma-Aldrich) and sealed.

Sciatic nerves were post-fixed in 4% PFA for 24 h at 4° C., then transferred to PBS for 2 h at RT. Nerves were incubated in sucrose gradient (5%, 10%, 20%) for 30 mins at RT for 5% and 10%, and overnight at 4° C. in 20% sucrose in DPBS. Nerves were mounted in Tissue-Tek optimal cooling temperature (OCT) mounting medium (Sakura Finetek, Torrence, CA), cut in a cryostat at −20° C. at 14 μm thickness, mounted on microscope slides, and stored at −80° C. until staining. Staining was performed by warming slides briefly at RT and washing in Tris-buffered saline (TBS) prior to incubating in primary antibody diluted in antibody dilution buffer (ADB; TBS with 0.02% sodium azide and 0.2% Triton X-100) overnight at RT in a humidity chamber. Slides were washed thrice in TBS, then incubated in Alexa Fluor-conjugated secondary antibodies diluted in ADB for 1 h at RT, washed again 3× in TBS, mounted with PVA-DABCO, and sealed.

Visualization and imaging of brain sections and sciatic nerves were carried out either with Nikon Eclipse Ti2-E motorized inverted microscope and NIS-Elements imaging software, or with the Zeiss LSM 800 confocal microscope and Zeiss ZEN imaging software.

Western blot: Cell were lysed with radioimmunoprecipitation (RIPA) lysis and extraction buffer (25 mM Tris.HCl PH 7.6, 150 mM NaCl, 1% NP-40, 1% sodium deoxycholate, 0.1% SDS) containing 1× complete™, EDTA-free protease Inhibitor cocktail (Roche, Indianapolis, IN). Tissue samples were incubated in T-PER™ Tissue Protein Extraction Reagent (Thermo Scientific) with 1× cOmplete™, EDTA-free protease Inhibitor cocktail, then homogenized using QIAGEN TissueLyser II bead mill (QIAGEN, Valencia, CA) set at 30 Hz for 2 min at RT. Lysates were sonicated for 3-5 s then centrifuged at 5000×g for 20 mins at 4° C. The supernatant containing total protein was quantified via a detergent compatible (DC) protein assay kit (Bio-Rad Laboratories, Hercules, CA) following manufacturer protocols. Forty micrograms of cell/tissue lysate were loaded onto 4-12% Bis-Tris polyacrylamide gels (Invitrogen) and run at 120 V for 1 h. Protein was transferred onto a PDVF membrane and blocked for 1 h at RT with Intercept® (PBS) Blocking Buffer (LI-COR Biosciences, Lincoln, NE). Primary antibodies for NF2 (ABclonal A2456, 1:2000 dilution), GFP (Abcam ab13970, 1:1000 dilution), GAPDH (Millipore MAB374, 1:5000 dilution), and α-tubulin (Sigma-Aldrich T6074, 1:5000 dilution) were diluted in 1:1 blocking buffer/TBST with 1% Tween-20 (TBS-T) and incubated overnight at 4° C. The membrane was then washed three times with TBS-T and incubated with LI-COR IRDye 800 or 680 secondary antibodies (1:7000 dilution) diluted in 1:1 blocking buffer/TBS-T with 0.2% sodium dodecyl sulfate (SDS) for 1 h at RT. Blots were washed again thrice with TBS-T before imaging using Odyssey DLx Imaging System (LI-COR) and analysis with Image Studio™ Lite Quantification Software (LI-COR). GAPDH or α-tubulin was used as housekeeping genes to normalize NF2 or GFP expression.

Quantitative PCR: Total RNA was isolated from cell pellets using TRIzol (Invitrogen) extraction protocol. RNA samples were then subjected to DNAse-I treatment using QIAGEN RNase-Free DNase Set. RNA quality and concentration was determined by NanoDrop spectrophotometric analysis (Thermo Scientific). First-strand cDNA was generated with 1000 μg of RNA per setup using RevertAid RT Reverse Transcription Kit (Thermo Scientific) following manufacturer protocols. Quantitative PCR (qPCR) was performed using 1 μL of RT product, 500 nM of primers, 1× Power SYBR® Green PCR Master Mix (Applied Biosystems), and nuclease-free water to a total volume of 20 μL. PCR was performed in a 96-well plate qPCR system (QuantStudio 3, Applied Biosystems) with cycling conditions of 50° C. for 2 min, 95° C. for 10 min, 40 cycles of 95° C. for 15 s, and 60° C. for 1 min. Gene-specific primers used are listed in Table 3 below. Each sample was run in triplicate and relative mRNA expression was calculated using the comparative Ct (ΔΔCT) method with endogenous GAPDH and RPL13A as housekeeping controls for normalization. At least 3 biological replicates were performed for each experiment.

Cell proliferation assay (MTT): U87 cells were re-seeded in 96-well plates in triplicate at 1500 cells/well upon AAV transduction as described above, in DMEM supplemented with 5% FBS and 1% antibiotic-antimycotic. The number of metabolically active cells per well was measured at 48 h after seeding using Cell Proliferation Kit I (MTT) (Roche) by adding 10 μL of MTT labeling reagent per well, incubating at 37° C. for 4 h, adding 10 μL of solubilization buffer, and overnight incubation at 37° C. Absorbance values at 575 nm were measured with a colorimetric plate reader (Synergy 2; BioTek, Winooski, VT) and analyzed using Gen5 data collection and analysis software (BioTek). Mean absorbances were calculated per setup and normalized against NA only treated cells.

Statistical analysis: All statistical tests were performed by unpaired two-tailed t-test to measure differences between two setups. Data from all quantitative experiments are presented as the mean±standard error (SEM). In all tests, significance value was defined as *P<0.05, **P<0.01, ***P<0.001, and ****P<0.0001. Graphs were generated using GraphPad Prism 9.0 software.

TABLE 1
Primers used for amplifying and cloning
truncated NF2 promoter sequences and NF2
isoform 1 cDNA

| | | Restric- |
|---|---|---|
| PRIMER | SEQUENCE | tion |
| NAME | (5′-3′) | Enzyme |
| NF2 2381 | TTT TTT GGT ACC CCC GGT | KpnI |
| Fw KpnI | GTC ACG TAT TTT TAT C | |
| | (SEQ ID NO: 23) | |
| NF2 2381 | TTT TTT CTG CAG CTG CGC | PstI |
| Rv PstI | GGG TCC CCA GCG | |
| | (SEQ ID NO: 24) | |
| NF2 400 | TTT TTT GGT ACC AAG AGT | KpnI |
| Fw KpnI | CCC TCC CTG GGC CAA C | |
| | (SEQ ID NO: 25) | |
| NF2 400 | TTT TTT CTG CAG GCG CGC | PstI |
| Rv PstI | CCG CGG GAA GCG | |
| | (SEQ ID NO: 26) | |
| NF2-cDNA- | TTTTT ACCGGT | AgeI |
| AgeI-Fwd | TAAAGGGCTCAGAGTGCAGG | |
| | (SEQ ID NO: 27) | |
| NF2-cDNA- | TTTTT TGTACA | BsrGI |
| BsrGI-Rev | GTAGCAGGAGAAGTGGCAGG | |
| | (SEQ ID NO: 28) | |

TABLE 2
Antibodies used for Immunostaining

| Target | Provider | Cat. No. | Host species | Dilution |
|---|---|---|---|---|
| NF2 | Sigma-Aldrich | HPA003097 | Rabbit | 1:500 (tissue) |
| Total NF2 | Abcam | ab88957 | Mouse | 1:50 (cells) |
| p-NF2 S518 | Abcam | ab2478 | Rabbit | 1:50 |
| GFP | Abcam | ab13970 | Chicken | 1:500 (cells, |
| | | | | tissue) |
| S100β | Swant | 371 | Rabbit | 1:500 |
| MBP | Abcam | ab40390 | Rabbit | 1:500 (tissue) |
| MBP | EnCor | CPCA-MBP | Chicken | 1:1000 (tissue), |
| | | | | 1:200 (cells) |
| KROX20 | Novus Biologicals | NB110-59723 | Rabbit | 1:50 |
| c-Myc | Millipore | 06-340 | Rabbit | 1:100 |
| Sox2 | Sigma-Aldrich | AB5603 | Rabbit | 1:100 |

TABLE 3
Gene-specific primers used for qPCR

| | Sequence | SEQ |
|---|---|---|
| Primer | | ID |
| name | (5′-3′) | NO: |
| Total | TGCGAGATGAAGTGGAAAGG | 29 |
| NF2-Fwd | | |
| Total | GCCAAGAAGTGAAAGGTGAC | 30 |
| NF2-Rev | | |
| NF2 | CCTTGAAACTGAAAGAGAGG | 31 |
| isoform | | |
| 1-Fwd | | |
| NF2 | CAAGGTGAGCTTTTTAATGG | 32 |
| isoform | | |
| 1-Rev | | |
| Vector- | TGGTGGTGCAAATCAAAGAA | 33 |
| specific | | |
| NF2-Fwd | | |
| Vector- | CGATCCTCACGGTGAACG | 34 |
| specific | | |
| NF2-Rev | | |
| | FIBROBLAST MARKERS | |
| FN1-Fwd | CAAGCCAGATGTCAGAAGC | 35 |
| FN1-Rev | GGATGGTGCATCAATGGCA | 36 |
| Col3a1- | CCCTGGATCTCCAGGATAC | 37 |
| Fwd | | |
| Col3a1- | GTCCGGGTCTACCTGATTC | 38 |
| Rev | | |
| | SCHWANN CELL MARKERS | |
| KROX20- | CACCAGCTGTCTGACAACATCTAC | 39 |
| Fwd | | |
| KROX20- | CCTGCACAGCCAGAATAAGG | 40 |
| Rev | | |
| MBP-Fwd | TTAAGCTGGGAGGAAGAGATAGT | 41 |
| MBP-Rev | GGGAGGGGTCATCTGCTCTA | 42 |
| | HOUSEKEEPING GENES | |
| GAPDH- | CAACTTTGGTATCGTGGAAGGAC | 43 |
| Fwd | | |
| GAPDH- | ACAGTCTTCTGGATGGCAGTG | 44 |
| Rev | | |
| RPL13A- | CCTGGAGGAGAAGAGGAAAGAGA | 45 |
| Fwd | | |
| RPL13A- | TTGAGGACCTCTGTGTATTTGTCAA | 46 |
| Rev | | |

While the present invention has been described in terms of specific embodiments, it is understood that variations and modifications will occur to those skilled in the art. Accordingly, only such limitations as appear in the claims should be placed on the invention.

All documents referred to in this application are hereby incorporated by reference in their entirety.

What is claimed:

1. A method of delivering a transgene encoding a Merlin protein with tumor suppressor activity to a subject in need of Merlin protein tumor suppressor activity, wherein the method comprises administering a gene therapy vector comprising the transgene to the subject by intravenous delivery, intratumoral delivery or intrathecal delivery.

2. The method of claim 1 comprising delivering a transgene encoding a Merlin protein with tumor suppressor activity to a subject with at least one defective NF2 allelle, wherein the method comprises administering a gene therapy vector comprising the transgene to the subject by intravenous delivery, intratumoral delivery or intrathecal delivery.

3. The method of claim 2 wherein the transgene is delivered to Schwann cells or Meningeal cells, or both, of the subject.

4. The method of claim 3 wherein the administration treats a malignant glioblastoma in the subject.

5. The method of claim 2 or 3 wherein the administration treats Neurofibromatosis Type 2 in the subject.

6. The method of claim 5 wherein the administration alleviates one or more of tumors, hearing loss, tinnitus, balance problems, facial weakness or numbness, visual impairment, cataract, seizure and brainstem compression in the subject.

7. The method of claim 6 wherein the tumors are one or more of schwannomas, meningiomas, ependymomas, or other cranial nerve tumors.

8. The method of any one of claims 1-7 wherein the transgene comprises the polynucleotide of SEQ ID NO: 1, 6, 7, 8, or 9, or a polynucleotide at least 90%, 91%, 92%, 93%, 94%, 95%, 96%, 97%, 98%, or 99% identical to SEQ ID NO: 1, 6, 7, 8 or 9 that encodes a Merlin protein with tumor suppressor activity.

9. The method of any one of claims 1-7 wherein the transgene comprises the polynucleotide of SEQ ID NO: 16, 17, 18, 19, 20, or a polynucleotide at least 90%, 91%, 92%, 93%, 94%, 95%, 96%, 97%, 98%, or 99% identical to SEQ ID NO: 16, 17, 18, 19, 20, that encodes a phosphorylation-resistant Merlin protein with tumor suppressor activity.

10. The method of any one of claims 1-9 wherein the gene therapy vector is an AAV1, AAV2, AAV3, AAV4, AAV5, AAV6, AAV7, AAV8, AAV9, AAVRH10, AAVRH74, AAV11, AAV12, AAV13, AAVTT, Anc80 or AAV7m8 serotype vector, or a derivative thereof.

11. The method of any one of claims 1-10, wherein the gene therapy vector is administered by intrathecal delivery.

12. The method of any one of claims 1-11 wherein the gene therapy vector is administered by intrathecal delivery, and the method further comprises placing the subject in the Trendelenburg position after administration of the gene therapy vector.

13. The method of any one of claims 1-10, wherein the gene therapy vector is administered by intratumoral delivery.

14. The method of any one of claims 1-10, wherein the gene therapy vector is administered by intravenous delivery.

15. The method of any one of claims 1-10, wherein the gene therapy vector is administered by intrathecal delivery.

16. The method of any one of claims 1-15, wherein the gene therapy vector is administered by intrathecal delivery, and the method further comprises placing the subject in the Trendelenburg position after administration of the gene therapy vector.

17. A transgene comprising the polynucleotide of SEQ ID NO: 1, 6, 7, 8, or 9, or a polynucleotide at least 90%, 91%, 92%, 93%, 94%, 95%, 96%, 97%, 98%, or 99% identical to SEQ ID NO: 1, 6, 7, 8 or 9 that encodes a Merlin protein with tumor suppressor activity.

18. A transgene comprising the polynucleotide of SEQ ID NO: 16, 17, 18, 19, 20, or a polynucleotide at least 90%, 91%, 92%, 93%, 94%, 95%, 96%, 97%, 98%, or 99% identical to SEQ ID NO: 16, 17, 18, 19, 20, that encodes a phosphorylation-resistant Merlin protein with tumor suppressor activity.

19. A recombinant adeno-associated virus (rAAV) with a genome comprising a transgene comprising the polynucleotide of SEQ ID NO: 1, 6, 7, 8, or 9, or a polynucleotide at least 90%, 91%, 92%, 93%, 94%, 95%, 96%, 97%, 98%, or 99% identical to SEQ ID NO: 1, 6, 7, 8 or 9 that encodes a Merlin protein with tumor suppressor activity.

20. A recombinant adeno-associate virus (rAAV) with a genome comprising a transgene comprises the polynucleotide of SEQ ID NO: 16, 17, 18, 19, 20, or a polynucleotide at least 90%, 91%, 92%, 93%, 94%, 95%, 96%, 97%, 98%, or 99% identical to SEQ ID NO: 16, 17, 18, 19, 20, that encodes a phosphorylation-resistant Merlin protein with tumor suppressor activity.

21. The recombinant adeno-associated virus (rAAV) of claim 19 or 20 wherein the recombinant adeno-associate virus is an AAV1, AAV2, AAV3, AAV4, AAV5, AAV6, AAV7, AAV8, AAV9, AAVRH10, AAVRH74, AAV11, AAV12, AAV13, AAVTT, Anc80 or AAV7m8 serotype vector, or a derivative thereof.

22. The recombinant adeno-associate virus (rAAV) of claim 21 that is an AAV9.


SEQUENCE LISTING (47 sequences)

SEQ ID NO: 1 - DNA, 1888 bp, Artificial Sequence
  Synthetic
  misc_feature: Neurofibromin 2/Merlin polynucleotide  - NF2 Construct
aaagggctcagagtgcaggccgtggggcgcgagggtcccgggcctgagccccgcgccatggccggggccatcgcttcccgcatgagcttcagctctctcaagaggaagcaacccaagacgttcaccgtgaggatcgtcaccatggacgccgagatggagttcaattgcgagatgaagtggaaagggaaggacctctttgatttggtgtgccggactctggggctccgagaaacctggttctttggactgcagtacacaatcaaggacacagtggcctggctcaaaatggacaagaaggtactggatcatgatgtttcaaaggaagaaccagtcacctttcacttcttggccaaattttatcctgagaatgctgaagaggagctggttcaggagatcacacaacatttattcttcttacaggtaaagaagcagattttagatgaaaagatctactgccctcctgaggcttctgtgctcctggcttcttacgccgtccaggccaagtatggtgactacgaccccagtgttcacaagcggggatttttggcccaagaggaattgcttccaaaaagggtaataaatctgtatcagatgactccggaaatgtgggaggagagaattactgcttggtacgcagagcaccgaggccgagccagggatgaagctgaaatggaatatctgaagatagctcaggacctggagatgtacggtgtgaactactttgcaatccggaataaaaagggcacagagctgctgcttggagtggatgccctggggcttcacatttatgaccctgagaacagactgacccccaagatctccttcccgtggaatgaaatccgaaacatctcgtacagtgacaaggagtttactattaaaccactggataagaaaattgatgtcttcaagtttaactcctcaaagcttcgtgttaataagctgattctccagctatgtatcgggaaccatgatctatttatgaggagaaggaaagccgattctttggaagttcagcagatgaaagcccaggccagggaggagaaggctagaaagcagatggagcggcagcgcctcgctcgagagaagcagatgagggaggaggctgaacgcacgagggatgagttggagaggaggctgctgcagatgaaagaagaagcaacaatggccaacgaagcactgatgcggtctgaggagacagctgacctgttggctgaaaaggcccagatcaccgaggaggaggcaaaacttctggcccagaaggccgcagaggctgagcaggaaatgcagcgcatcaaggccacagcgattcgcacggaggaggagaagcgcctgatggagcagaaggtgctggaagccgaggtgctggcactgaagatggctgaggagtcagagaggagggccaaagaggcagatcagctgaagcaggacctgcaggaagcacgcgaggcggagcgaagagccaagcagaagctcctggagattgccaccaagcccacgtacccgcccatgaacccaattccagcaccgttgcctcctgacataccaagcttcaacctcattggtgacagcctgtctttcgacttcaaagatactgacatgaagcggctttccatggagatagagaaagaaaaagtggaatacatggaaaagagcaagcatctgcaggagcagctcaatgaactcaagacagaaatcgaggccttgaaactgaaagagagggagacagctctggatattctgcacaatgagaactccgacaggggtggcagcagcaagcacaataccattaaaaagctcaccttgcagagcgccaagtcccgagtggccttctttgaagagctctagcaggtgacccagccaccccaggacctgccacttctcctgctac

SEQ ID NO: 2 - DNA, 2301 bp, Artificial Sequence
  Synthetic
  misc_feature: NF2-2.3kb promoter
cccggtgtcacgtatttttatcagcagccatttgttgtccctgtggggagcccagaaaacccagcccggttctcccacctgcggggaagaaatgaagcaatgataatgaagacactcccatgccaagcccatactgccctgagcaatcagttttggtggggagaatttgctagcagaaggcaggacataaagtccccacagcagcctcactgggtgtggcagcaaatcattcctgaggcctggccggtgctaccaggcacagcaggggtccgtgcagaagaactagatttattccattaatataccgttacagcaacaatgtctgctgagtactccggaactgtaattcacatttcctgaggctgtagtagggagaatttggaagcagggcagcacagtgctaaccagcaggctctgggtgggacagacctgggtttgagtcctagctttgcctttcactatctgcattatagtcagcgagtggctctatcatatctctcagtatcttcacctgtggagttgggggggtgatgctaatgttagctaacatttatggggtgattattgttgttaaaactcagtacaccgggcgcggtggctcacgcctgtaatcccagcactttgggaggccaaggtgggcagatcacctgaggtcagaagtttgagaccagcctggctaacatggtgaaaccccgtccctactaaaaatacaaaaattagccgggcgtggtggcgggtgcctgtaatcccaactactggggaggctgaggcaggagaattgcttgaacccgggaggcggaggttgcagagagccgagaccgccccattgtactccagcctgggcaacaagagcgaaactctgtctcaaaacaaaaaacaaacgaaaaaacacagtaaattgtccctatttgactactaagagctattttaaaatagaatttattatctaagtacagaaaggtgtaattatcccccctacacccgttagaacccttgatgacactaaataaataatacatgaatacggttcaaaaatgggcatcatagagtataaaataaagaatgctttcattttcaacccaaattcagcaatttcataggatgccaggctcagctctaaagccttatatgtattcagtgcgctgaaattcaacaacctcacgaggtaggtataaccaataataattctaggcggggcgtggtggctcacgcctgtaatcctagcacttcgggaggccgaggcgggcggatcacctgaggttcgagaccagcctgacgaacatggagaaaccccgtctctaataaaaatacaaaattagccgggcgtggtgacgcatacctgtaatcccagctactcgggaggctgacgccggaaaatcgcttgaacccgggaggcggaggttgcagtgagccgagatcgcgccactgcactccagcctgggcaacaagagcgaaactccgtcccaataataataataattaataataataataataacaataataataataattctaaagggaagcagagggatctggcgggaaagtgccagcaaacctaacaggcctccacaagtgtctttaaagtttcataggggtcggtatatgtgattaactgccaaagaagaatattcgccgtgtgtccgaaacgcagtcccctggggcgccacactcggagcctacttgaaccgaagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgcggagtgaggacggtgacagccacgcgcgcgcgtacgcgcccgatgcagcgcggccccgtgaccctagtcggccgctgagaggcgcgcggagtctgggccgctgccgtctaggggtcccgtcccgaggcgtccccggcatctccggcccgaatcccggagtgccgggtcgcgcctgcaccgaaggtcccggctcctgtgccctccctgcag

SEQ ID NO: 3 - DNA, 400 bp, Artificial Sequence
  Synthetic
  misc_feature: NF2-400bp promoter
aagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgc

SEQ ID NO: 4 - DNA, 610 bp, Artificial Sequence
  Synthetic
  misc_feature: NF2-610bp promoter
aagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgcggagtgaggacggtgacagccacgcgcgcgcgtacgcgcccgatgcagcgcggccccgtgaccctagtcggccgctgagaggcgcgcggagtctgggccgctgccgtctaggggtcccgtcccgaggcgtccccggcatctccggcccgaatcccggagtgccgggtcgcgcctgcaccgaaggtcccggctcctgtgccctccctgcag

SEQ ID NO: 5 - DNA, 2091 bp, Artificial Sequence
  Synthetic
  misc_feature: NF2-2.1kb promoter
cccggtgtcacgtatttttatcagcagccatttgttgtccctgtggggagcccagaaaacccagcccggttctcccacctgcggggaagaaatgaagcaatgataatgaagacactcccatgccaagcccatactgccctgagcaatcagttttggtggggagaatttgctagcagaaggcaggacataaagtccccacagcagcctcactgggtgtggcagcaaatcattcctgaggcctggccggtgctaccaggcacagcaggggtccgtgcagaagaactagatttattccattaatataccgttacagcaacaatgtctgctgagtactccggaactgtaattcacatttcctgaggctgtagtagggagaatttggaagcagggcagcacagtgctaaccagcaggctctgggtgggacagacctgggtttgagtcctagctttgcctttcactatctgcattatagtcagcgagtggctctatcatatctctcagtatcttcacctgtggagttgggggggtgatgctaatgttagctaacatttatggggtgattattgttgttaaaactcagtacaccgggcgcggtggctcacgcctgtaatcccagcactttgggaggccaaggtgggcagatcacctgaggtcagaagtttgagaccagcctggctaacatggtgaaaccccgtccctactaaaaatacaaaaattagccgggcgtggtggcgggtgcctgtaatcccaactactggggaggctgaggcaggagaattgcttgaacccgggaggcggaggttgcagagagccgagaccgccccattgtactccagcctgggcaacaagagcgaaactctgtctcaaaacaaaaaacaaacgaaaaaacacagtaaattgtccctatttgactactaagagctattttaaaatagaatttattatctaagtacagaaaggtgtaattatcccccctacacccgttagaacccttgatgacactaaataaataatacatgaatacggttcaaaaatgggcatcatagagtataaaataaagaatgctttcattttcaacccaaattcagcaatttcataggatgccaggctcagctctaaagccttatatgtattcagtgcgctgaaattcaacaacctcacgaggtaggtataaccaataataattctaggcggggcgtggtggctcacgcctgtaatcctagcacttcgggaggccgaggcgggcggatcacctgaggttcgagaccagcctgacgaacatggagaaaccccgtctctaataaaaatacaaaattagccgggcgtggtgacgcatacctgtaatcccagctactcgggaggctgacgccggaaaatcgcttgaacccgggaggcggaggttgcagtgagccgagatcgcgccactgcactccagcctgggcaacaagagcgaaactccgtcccaataataataataattaataataataataataacaataataataataattctaaagggaagcagagggatctggcgggaaagtgccagcaaacctaacaggcctccacaagtgtctttaaagtttcataggggtcggtatatgtgattaactgccaaagaagaatattcgccgtgtgtccgaaacgcagtcccctggggcgccacactcggagcctacttgaaccgaagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgc

SEQ ID NO: 6 - DNA, 2483 bp, Artificial Sequence
  Synthetic
  misc_feature: NF2-400bp- NF2 Expression cassette
  misc_feature (1)..(400): NF2 400bp Promoter
  misc_feature (413)..(512): SV40 Intron
  misc_feature (530)..(2417): NF2
  misc_feature (2435)..(2483): Poly A
aagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgcctgcagataactggtaagtttagtcttttttgtcttttatttcaggtcccggatccggtggtggtgcaaatcaaagaactgctcctcagtcgatgttgcctttacttctaggcctgtacggaaaccggtaaagggctcagagtgcaggccgtggggcgcgagggtcccgggcctgagccccgcgccatggccggggccatcgcttcccgcatgagcttcagctctctcaagaggaagcaacccaagacgttcaccgtgaggatcgtcaccatggacgccgagatggagttcaattgcgagatgaagtggaaagggaaggacctctttgatttggtgtgccggactctggggctccgagaaacctggttctttggactgcagtacacaatcaaggacacagtggcctggctcaaaatggacaagaaggtactggatcatgatgtttcaaaggaagaaccagtcacctttcacttcttggccaaattttatcctgagaatgctgaagaggagctggttcaggagatcacacaacatttattcttcttacaggtaaagaagcagattttagatgaaaagatctactgccctcctgaggcttctgtgctcctggcttcttacgccgtccaggccaagtatggtgactacgaccccagtgttcacaagcggggatttttggcccaagaggaattgcttccaaaaagggtaataaatctgtatcagatgactccggaaatgtgggaggagagaattactgcttggtacgcagagcaccgaggccgagccagggatgaagctgaaatggaatatctgaagatagctcaggacctggagatgtacggtgtgaactactttgcaatccggaataaaaagggcacagagctgctgcttggagtggatgccctggggcttcacatttatgaccctgagaacagactgacccccaagatctccttcccgtggaatgaaatccgaaacatctcgtacagtgacaaggagtttactattaaaccactggataagaaaattgatgtcttcaagtttaactcctcaaagcttcgtgttaataagctgattctccagctatgtatcgggaaccatgatctatttatgaggagaaggaaagccgattctttggaagttcagcagatgaaagcccaggccagggaggagaaggctagaaagcagatggagcggcagcgcctcgctcgagagaagcagatgagggaggaggctgaacgcacgagggatgagttggagaggaggctgctgcagatgaaagaagaagcaacaatggccaacgaagcactgatgcggtctgaggagacagctgacctgttggctgaaaaggcccagatcaccgaggaggaggcaaaacttctggcccagaaggccgcagaggctgagcaggaaatgcagcgcatcaaggccacagcgattcgcacggaggaggagaagcgcctgatggagcagaaggtgctggaagccgaggtgctggcactgaagatggctgaggagtcagagaggagggccaaagaggcagatcagctgaagcaggacctgcaggaagcacgcgaggcggagcgaagagccaagcagaagctcctggagattgccaccaagcccacgtacccgcccatgaacccaattccagcaccgttgcctcctgacataccaagcttcaacctcattggtgacagcctgtctttcgacttcaaagatactgacatgaagcggctttccatggagatagagaaagaaaaagtggaatacatggaaaagagcaagcatctgcaggagcagctcaatgaactcaagacagaaatcgaggccttgaaactgaaagagagggagacagctctggatattctgcacaatgagaactccgacaggggtggcagcagcaagcacaataccattaaaaagctcaccttgcagagcgccaagtcccgagtggccttctttgaagagctctagcaggtgacccagccaccccaggacctgccacttctcctgctactgtacaagtaaaagcttaataaaagatctttattttcattagatctgtgtgttggttttttgtgtg

SEQ ID NO: 7 - DNA, 2687 bp, Artificial Sequence
  Synthetic
  misc_feature: NF2-610bp- NF2 Expression cassette
  misc_feature (1)..(610): NF2 610bp Promoter
  misc_feature (617)..(716): SV40 Intron
  misc_feature (734)..(2621): NF2
  misc_feature (2639)..(2687): Poly A
aagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgcggagtgaggacggtgacagccacgcgcgcgcgtacgcgcccgatgcagcgcggccccgtgaccctagtcggccgctgagaggcgcgcggagtctgggccgctgccgtctaggggtcccgtcccgaggcgtccccggcatctccggcccgaatcccggagtgccgggtcgcgcctgcaccgaaggtcccggctcctgtgccctccctgcagataactggtaagtttagtcttttttgtcttttatttcaggtcccggatccggtggtggtgcaaatcaaagaactgctcctcagtcgatgttgcctttacttctaggcctgtacggaaaccggtaaagggctcagagtgcaggccgtggggcgcgagggtcccgggcctgagccccgcgccatggccggggccatcgcttcccgcatgagcttcagctctctcaagaggaagcaacccaagacgttcaccgtgaggatcgtcaccatggacgccgagatggagttcaattgcgagatgaagtggaaagggaaggacctctttgatttggtgtgccggactctggggctccgagaaacctggttctttggactgcagtacacaatcaaggacacagtggcctggctcaaaatggacaagaaggtactggatcatgatgtttcaaaggaagaaccagtcacctttcacttcttggccaaattttatcctgagaatgctgaagaggagctggttcaggagatcacacaacatttattcttcttacaggtaaagaagcagattttagatgaaaagatctactgccctcctgaggcttctgtgctcctggcttcttacgccgtccaggccaagtatggtgactacgaccccagtgttcacaagcggggatttttggcccaagaggaattgcttccaaaaagggtaataaatctgtatcagatgactccggaaatgtgggaggagagaattactgcttggtacgcagagcaccgaggccgagccagggatgaagctgaaatggaatatctgaagatagctcaggacctggagatgtacggtgtgaactactttgcaatccggaataaaaagggcacagagctgctgcttggagtggatgccctggggcttcacatttatgaccctgagaacagactgacccccaagatctccttcccgtggaatgaaatccgaaacatctcgtacagtgacaaggagtttactattaaaccactggataagaaaattgatgtcttcaagtttaactcctcaaagcttcgtgttaataagctgattctccagctatgtatcgggaaccatgatctatttatgaggagaaggaaagccgattctttggaagttcagcagatgaaagcccaggccagggaggagaaggctagaaagcagatggagcggcagcgcctcgctcgagagaagcagatgagggaggaggctgaacgcacgagggatgagttggagaggaggctgctgcagatgaaagaagaagcaacaatggccaacgaagcactgatgcggtctgaggagacagctgacctgttggctgaaaaggcccagatcaccgaggaggaggcaaaacttctggcccagaaggccgcagaggctgagcaggaaatgcagcgcatcaaggccacagcgattcgcacggaggaggagaagcgcctgatggagcagaaggtgctggaagccgaggtgctggcactgaagatggctgaggagtcagagaggagggccaaagaggcagatcagctgaagcaggacctgcaggaagcacgcgaggcggagcgaagagccaagcagaagctcctggagattgccaccaagcccacgtacccgcccatgaacccaattccagcaccgttgcctcctgacataccaagcttcaacctcattggtgacagcctgtctttcgacttcaaagatactgacatgaagcggctttccatggagatagagaaagaaaaagtggaatacatggaaaagagcaagcatctgcaggagcagctcaatgaactcaagacagaaatcgaggccttgaaactgaaagagagggagacagctctggatattctgcacaatgagaactccgacaggggtggcagcagcaagcacaataccattaaaaagctcaccttgcagagcgccaagtcccgagtggccttctttgaagagctctagcaggtgacccagccaccccaggacctgccacttctcctgctactgtacaagtaaaagcttaataaaagatctttattttcattagatctgtgtgttggttttttgtgtg

SEQ ID NO: 8 - DNA, 4174 bp, Artificial Sequence
  Synthetic
  misc_feature: NF2-2.1kb- NF2 Expression cassette
  misc_feature (1)..(2091): NF2 2.1kb Promoter
  misc_feature (2104)..(2203): SV40 Intron
  misc_feature (2221)..(4108): NF2
  misc_feature (4126)..(4174): Poly A
cccggtgtcacgtatttttatcagcagccatttgttgtccctgtggggagcccagaaaacccagcccggttctcccacctgcggggaagaaatgaagcaatgataatgaagacactcccatgccaagcccatactgccctgagcaatcagttttggtggggagaatttgctagcagaaggcaggacataaagtccccacagcagcctcactgggtgtggcagcaaatcattcctgaggcctggccggtgctaccaggcacagcaggggtccgtgcagaagaactagatttattccattaatataccgttacagcaacaatgtctgctgagtactccggaactgtaattcacatttcctgaggctgtagtagggagaatttggaagcagggcagcacagtgctaaccagcaggctctgggtgggacagacctgggtttgagtcctagctttgcctttcactatctgcattatagtcagcgagtggctctatcatatctctcagtatcttcacctgtggagttgggggggtgatgctaatgttagctaacatttatggggtgattattgttgttaaaactcagtacaccgggcgcggtggctcacgcctgtaatcccagcactttgggaggccaaggtgggcagatcacctgaggtcagaagtttgagaccagcctggctaacatggtgaaaccccgtccctactaaaaatacaaaaattagccgggcgtggtggcgggtgcctgtaatcccaactactggggaggctgaggcaggagaattgcttgaacccgggaggcggaggttgcagagagccgagaccgccccattgtactccagcctgggcaacaagagcgaaactctgtctcaaaacaaaaaacaaacgaaaaaacacagtaaattgtccctatttgactactaagagctattttaaaatagaatttattatctaagtacagaaaggtgtaattatcccccctacacccgttagaacccttgatgacactaaataaataatacatgaatacggttcaaaaatgggcatcatagagtataaaataaagaatgctttcattttcaacccaaattcagcaatttcataggatgccaggctcagctctaaagccttatatgtattcagtgcgctgaaattcaacaacctcacgaggtaggtataaccaataataattctaggcggggcgtggtggctcacgcctgtaatcctagcacttcgggaggccgaggcgggcggatcacctgaggttcgagaccagcctgacgaacatggagaaaccccgtctctaataaaaatacaaaattagccgggcgtggtgacgcatacctgtaatcccagctactcgggaggctgacgccggaaaatcgcttgaacccgggaggcggaggttgcagtgagccgagatcgcgccactgcactccagcctgggcaacaagagcgaaactccgtcccaataataataataattaataataataataataacaataataataataattctaaagggaagcagagggatctggcgggaaagtgccagcaaacctaacaggcctccacaagtgtctttaaagtttcataggggtcggtatatgtgattaactgccaaagaagaatattcgccgtgtgtccgaaacgcagtcccctggggcgccacactcggagcctacttgaaccgaagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgcctgcagataactggtaagtttagtcttttttgtcttttatttcaggtcccggatccggtggtggtgcaaatcaaagaactgctcctcagtcgatgttgcctttacttctaggcctgtacggaaaccggtaaagggctcagagtgcaggccgtggggcgcgagggtcccgggcctgagccccgcgccatggccggggccatcgcttcccgcatgagcttcagctctctcaagaggaagcaacccaagacgttcaccgtgaggatcgtcaccatggacgccgagatggagttcaattgcgagatgaagtggaaagggaaggacctctttgatttggtgtgccggactctggggctccgagaaacctggttctttggactgcagtacacaatcaaggacacagtggcctggctcaaaatggacaagaaggtactggatcatgatgtttcaaaggaagaaccagtcacctttcacttcttggccaaattttatcctgagaatgctgaagaggagctggttcaggagatcacacaacatttattcttcttacaggtaaagaagcagattttagatgaaaagatctactgccctcctgaggcttctgtgctcctggcttcttacgccgtccaggccaagtatggtgactacgaccccagtgttcacaagcggggatttttggcccaagaggaattgcttccaaaaagggtaataaatctgtatcagatgactccggaaatgtgggaggagagaattactgcttggtacgcagagcaccgaggccgagccagggatgaagctgaaatggaatatctgaagatagctcaggacctggagatgtacggtgtgaactactttgcaatccggaataaaaagggcacagagctgctgcttggagtggatgccctggggcttcacatttatgaccctgagaacagactgacccccaagatctccttcccgtggaatgaaatccgaaacatctcgtacagtgacaaggagtttactattaaaccactggataagaaaattgatgtcttcaagtttaactcctcaaagcttcgtgttaataagctgattctccagctatgtatcgggaaccatgatctatttatgaggagaaggaaagccgattctttggaagttcagcagatgaaagcccaggccagggaggagaaggctagaaagcagatggagcggcagcgcctcgctcgagagaagcagatgagggaggaggctgaacgcacgagggatgagttggagaggaggctgctgcagatgaaagaagaagcaacaatggccaacgaagcactgatgcggtctgaggagacagctgacctgttggctgaaaaggcccagatcaccgaggaggaggcaaaacttctggcccagaaggccgcagaggctgagcaggaaatgcagcgcatcaaggccacagcgattcgcacggaggaggagaagcgcctgatggagcagaaggtgctggaagccgaggtgctggcactgaagatggctgaggagtcagagaggagggccaaagaggcagatcagctgaagcaggacctgcaggaagcacgcgaggcggagcgaagagccaagcagaagctcctggagattgccaccaagcccacgtacccgcccatgaacccaattccagcaccgttgcctcctgacataccaagcttcaacctcattggtgacagcctgtctttcgacttcaaagatactgacatgaagcggctttccatggagatagagaaagaaaaagtggaatacatggaaaagagcaagcatctgcaggagcagctcaatgaactcaagacagaaatcgaggccttgaaactgaaagagagggagacagctctggatattctgcacaatgagaactccgacaggggtggcagcagcaagcacaataccattaaaaagctcaccttgcagagcgccaagtcccgagtggccttctttgaagagctctagcaggtgacccagccaccccaggacctgccacttctcctgctactgtacaagtaaaagcttaataaaagatctttattttcattagatctgtgtgttggttttttgtgtg

SEQ ID NO: 9 - DNA, 4378 bp, Artificial Sequence
  Synthetic
  misc_feature: NF2-2.3kb-NF2 Expression cassette
  misc_feature (1)..(2295): NF2 2.3kb Promoter
  misc_feature (2308)..(2407): SV40 Intron
  misc_feature (2425)..(4312): NF2
  misc_feature (4330)..(4378): Poly A
cccggtgtcacgtatttttatcagcagccatttgttgtccctgtggggagcccagaaaacccagcccggttctcccacctgcggggaagaaatgaagcaatgataatgaagacactcccatgccaagcccatactgccctgagcaatcagttttggtggggagaatttgctagcagaaggcaggacataaagtccccacagcagcctcactgggtgtggcagcaaatcattcctgaggcctggccggtgctaccaggcacagcaggggtccgtgcagaagaactagatttattccattaatataccgttacagcaacaatgtctgctgagtactccggaactgtaattcacatttcctgaggctgtagtagggagaatttggaagcagggcagcacagtgctaaccagcaggctctgggtgggacagacctgggtttgagtcctagctttgcctttcactatctgcattatagtcagcgagtggctctatcatatctctcagtatcttcacctgtggagttgggggggtgatgctaatgttagctaacatttatggggtgattattgttgttaaaactcagtacaccgggcgcggtggctcacgcctgtaatcccagcactttgggaggccaaggtgggcagatcacctgaggtcagaagtttgagaccagcctggctaacatggtgaaaccccgtccctactaaaaatacaaaaattagccgggcgtggtggcgggtgcctgtaatcccaactactggggaggctgaggcaggagaattgcttgaacccgggaggcggaggttgcagagagccgagaccgccccattgtactccagcctgggcaacaagagcgaaactctgtctcaaaacaaaaaacaaacgaaaaaacacagtaaattgtccctatttgactactaagagctattttaaaatagaatttattatctaagtacagaaaggtgtaattatcccccctacacccgttagaacccttgatgacactaaataaataatacatgaatacggttcaaaaatgggcatcatagagtataaaataaagaatgctttcattttcaacccaaattcagcaatttcataggatgccaggctcagctctaaagccttatatgtattcagtgcgctgaaattcaacaacctcacgaggtaggtataaccaataataattctaggcggggcgtggtggctcacgcctgtaatcctagcacttcgggaggccgaggcgggcggatcacctgaggttcgagaccagcctgacgaacatggagaaaccccgtctctaataaaaatacaaaattagccgggcgtggtgacgcatacctgtaatcccagctactcgggaggctgacgccggaaaatcgcttgaacccgggaggcggaggttgcagtgagccgagatcgcgccactgcactccagcctgggcaacaagagcgaaactccgtcccaataataataataattaataataataataataacaataataataataattctaaagggaagcagagggatctggcgggaaagtgccagcaaacctaacaggcctccacaagtgtctttaaagtttcataggggtcggtatatgtgattaactgccaaagaagaatattcgccgtgtgtccgaaacgcagtcccctggggcgccacactcggagcctacttgaaccgaagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgcggagtgaggacggtgacagccacgcgcgcgcgtacgcgcccgatgcagcgcggccccgtgaccctagtcggccgctgagaggcgcgcggagtctgggccgctgccgtctaggggtcccgtcccgaggcgtccccggcatctccggcccgaatcccggagtgccgggtcgcgcctgcaccgaaggtcccggctcctgtgccctccctgcagataactggtaagtttagtcttttttgtcttttatttcaggtcccggatccggtggtggtgcaaatcaaagaactgctcctcagtcgatgttgcctttacttctaggcctgtacggaaaccggtaaagggctcagagtgcaggccgtggggcgcgagggtcccgggcctgagccccgcgccatggccggggccatcgcttcccgcatgagcttcagctctctcaagaggaagcaacccaagacgttcaccgtgaggatcgtcaccatggacgccgagatggagttcaattgcgagatgaagtggaaagggaaggacctctttgatttggtgtgccggactctggggctccgagaaacctggttctttggactgcagtacacaatcaaggacacagtggcctggctcaaaatggacaagaaggtactggatcatgatgtttcaaaggaagaaccagtcacctttcacttcttggccaaattttatcctgagaatgctgaagaggagctggttcaggagatcacacaacatttattcttcttacaggtaaagaagcagattttagatgaaaagatctactgccctcctgaggcttctgtgctcctggcttcttacgccgtccaggccaagtatggtgactacgaccccagtgttcacaagcggggatttttggcccaagaggaattgcttccaaaaagggtaataaatctgtatcagatgactccggaaatgtgggaggagagaattactgcttggtacgcagagcaccgaggccgagccagggatgaagctgaaatggaatatctgaagatagctcaggacctggagatgtacggtgtgaactactttgcaatccggaataaaaagggcacagagctgctgcttggagtggatgccctggggcttcacatttatgaccctgagaacagactgacccccaagatctccttcccgtggaatgaaatccgaaacatctcgtacagtgacaaggagtttactattaaaccactggataagaaaattgatgtcttcaagtttaactcctcaaagcttcgtgttaataagctgattctccagctatgtatcgggaaccatgatctatttatgaggagaaggaaagccgattctttggaagttcagcagatgaaagcccaggccagggaggagaaggctagaaagcagatggagcggcagcgcctcgctcgagagaagcagatgagggaggaggctgaacgcacgagggatgagttggagaggaggctgctgcagatgaaagaagaagcaacaatggccaacgaagcactgatgcggtctgaggagacagctgacctgttggctgaaaaggcccagatcaccgaggaggaggcaaaacttctggcccagaaggccgcagaggctgagcaggaaatgcagcgcatcaaggccacagcgattcgcacggaggaggagaagcgcctgatggagcagaaggtgctggaagccgaggtgctggcactgaagatggctgaggagtcagagaggagggccaaagaggcagatcagctgaagcaggacctgcaggaagcacgcgaggcggagcgaagagccaagcagaagctcctggagattgccaccaagcccacgtacccgcccatgaacccaattccagcaccgttgcctcctgacataccaagcttcaacctcattggtgacagcctgtctttcgacttcaaagatactgacatgaagcggctttccatggagatagagaaagaaaaagtggaatacatggaaaagagcaagcatctgcaggagcagctcaatgaactcaagacagaaatcgaggccttgaaactgaaagagagggagacagctctggatattctgcacaatgagaactccgacaggggtggcagcagcaagcacaataccattaaaaagctcaccttgcagagcgccaagtcccgagtggccttctttgaagagctctagcaggtgacccagccaccccaggacctgccacttctcctgctactgtacaagtaaaagcttaataaaagatctttattttcattagatctgtgtgttggttttttgtgtg

SEQ ID NO: 10 - DNA, 1312 bp, Artificial Sequence
  Synthetic
  misc_feature: NF2-400bp- GFP Expression cassette
  misc_feature (1)..(400): NF2 400bp Promoter
  misc_feature (413)..(512): SV40 Intron
  misc_feature (538)..(1257): GFP
  misc_feature (1264)..(1312): Poly A
aagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgcctgcagataactggtaagtttagtcttttttgtcttttatttcaggtcccggatccggtggtggtgcaaatcaaagaactgctcctcagtcgatgttgcctttacttctaggcctgtacggaaaccggtgtgttactatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttaataaaagatctttattttcattagatctgtgtgttggttttttgtgtg

SEQ ID NO: 11 - DNA, 1516 bp, Artificial Sequence
  Synthetic
  misc_feature: NF2-610bp- GFP Expression cassette
  misc_feature (1)..(610): NF2 610bp Promoter
  misc_feature (617)..(716): SV40 Intron
  misc_feature (742)..(1461): GFP
  misc_feature (1468)..(1516): Poly A
aagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgcggagtgaggacggtgacagccacgcgcgcgcgtacgcgcccgatgcagcgcggccccgtgaccctagtcggccgctgagaggcgcgcggagtctgggccgctgccgtctaggggtcccgtcccgaggcgtccccggcatctccggcccgaatcccggagtgccgggtcgcgcctgcaccgaaggtcccggctcctgtgccctccctgcagataactggtaagtttagtcttttttgtcttttatttcaggtcccggatccggtggtggtgcaaatcaaagaactgctcctcagtcgatgttgcctttacttctaggcctgtacggaaaccggtgtgttactatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttaataaaagatctttattttcattagatctgtgtgttggttttttgtgtg

SEQ ID NO: 12 - DNA, 3003 bp, Artificial Sequence
  Synthetic
  misc_feature: NF2-2.1kb- GFP Expression cassette
  misc_feature (1)..(2091): NF2 2.1kb Promoter
  misc_feature (2104)..(2203): SV40 Intron
  misc_feature (2229)..(2948): GFP
  misc_feature (2955)..(3003): Poly A
cccggtgtcacgtatttttatcagcagccatttgttgtccctgtggggagcccagaaaacccagcccggttctcccacctgcggggaagaaatgaagcaatgataatgaagacactcccatgccaagcccatactgccctgagcaatcagttttggtggggagaatttgctagcagaaggcaggacataaagtccccacagcagcctcactgggtgtggcagcaaatcattcctgaggcctggccggtgctaccaggcacagcaggggtccgtgcagaagaactagatttattccattaatataccgttacagcaacaatgtctgctgagtactccggaactgtaattcacatttcctgaggctgtagtagggagaatttggaagcagggcagcacagtgctaaccagcaggctctgggtgggacagacctgggtttgagtcctagctttgcctttcactatctgcattatagtcagcgagtggctctatcatatctctcagtatcttcacctgtggagttgggggggtgatgctaatgttagctaacatttatggggtgattattgttgttaaaactcagtacaccgggcgcggtggctcacgcctgtaatcccagcactttgggaggccaaggtgggcagatcacctgaggtcagaagtttgagaccagcctggctaacatggtgaaaccccgtccctactaaaaatacaaaaattagccgggcgtggtggcgggtgcctgtaatcccaactactggggaggctgaggcaggagaattgcttgaacccgggaggcggaggttgcagagagccgagaccgccccattgtactccagcctgggcaacaagagcgaaactctgtctcaaaacaaaaaacaaacgaaaaaacacagtaaattgtccctatttgactactaagagctattttaaaatagaatttattatctaagtacagaaaggtgtaattatcccccctacacccgttagaacccttgatgacactaaataaataatacatgaatacggttcaaaaatgggcatcatagagtataaaataaagaatgctttcattttcaacccaaattcagcaatttcataggatgccaggctcagctctaaagccttatatgtattcagtgcgctgaaattcaacaacctcacgaggtaggtataaccaataataattctaggcggggcgtggtggctcacgcctgtaatcctagcacttcgggaggccgaggcgggcggatcacctgaggttcgagaccagcctgacgaacatggagaaaccccgtctctaataaaaatacaaaattagccgggcgtggtgacgcatacctgtaatcccagctactcgggaggctgacgccggaaaatcgcttgaacccgggaggcggaggttgcagtgagccgagatcgcgccactgcactccagcctgggcaacaagagcgaaactccgtcccaataataataataattaataataataataataacaataataataataattctaaagggaagcagagggatctggcgggaaagtgccagcaaacctaacaggcctccacaagtgtctttaaagtttcataggggtcggtatatgtgattaactgccaaagaagaatattcgccgtgtgtccgaaacgcagtcccctggggcgccacactcggagcctacttgaaccgaagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgcctgcagataactggtaagtttagtcttttttgtcttttatttcaggtcccggatccggtggtggtgcaaatcaaagaactgctcctcagtcgatgttgcctttacttctaggcctgtacggaaaccggtgtgttactatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttaataaaagatctttattttcattagatctgtgtgttggttttttgtgtg

SEQ ID NO: 13 - DNA, 3207 bp, Artificial Sequence
  Synthetic
  misc_feature: NF2-2.3kb-GFP Expression cassette
  misc_feature (1)..(2301): NF2 2.3kb Promoter
  misc_feature (2308)..(2407): SV40 Intron
  misc_feature (2433)..(3152): GFP
  misc_feature (3159)..(3207): GFP CDS
cccggtgtcacgtatttttatcagcagccatttgttgtccctgtggggagcccagaaaacccagcccggttctcccacctgcggggaagaaatgaagcaatgataatgaagacactcccatgccaagcccatactgccctgagcaatcagttttggtggggagaatttgctagcagaaggcaggacataaagtccccacagcagcctcactgggtgtggcagcaaatcattcctgaggcctggccggtgctaccaggcacagcaggggtccgtgcagaagaactagatttattccattaatataccgttacagcaacaatgtctgctgagtactccggaactgtaattcacatttcctgaggctgtagtagggagaatttggaagcagggcagcacagtgctaaccagcaggctctgggtgggacagacctgggtttgagtcctagctttgcctttcactatctgcattatagtcagcgagtggctctatcatatctctcagtatcttcacctgtggagttgggggggtgatgctaatgttagctaacatttatggggtgattattgttgttaaaactcagtacaccgggcgcggtggctcacgcctgtaatcccagcactttgggaggccaaggtgggcagatcacctgaggtcagaagtttgagaccagcctggctaacatggtgaaaccccgtccctactaaaaatacaaaaattagccgggcgtggtggcgggtgcctgtaatcccaactactggggaggctgaggcaggagaattgcttgaacccgggaggcggaggttgcagagagccgagaccgccccattgtactccagcctgggcaacaagagcgaaactctgtctcaaaacaaaaaacaaacgaaaaaacacagtaaattgtccctatttgactactaagagctattttaaaatagaatttattatctaagtacagaaaggtgtaattatcccccctacacccgttagaacccttgatgacactaaataaataatacatgaatacggttcaaaaatgggcatcatagagtataaaataaagaatgctttcattttcaacccaaattcagcaatttcataggatgccaggctcagctctaaagccttatatgtattcagtgcgctgaaattcaacaacctcacgaggtaggtataaccaataataattctaggcggggcgtggtggctcacgcctgtaatcctagcacttcgggaggccgaggcgggcggatcacctgaggttcgagaccagcctgacgaacatggagaaaccccgtctctaataaaaatacaaaattagccgggcgtggtgacgcatacctgtaatcccagctactcgggaggctgacgccggaaaatcgcttgaacccgggaggcggaggttgcagtgagccgagatcgcgccactgcactccagcctgggcaacaagagcgaaactccgtcccaataataataataattaataataataataataacaataataataataattctaaagggaagcagagggatctggcgggaaagtgccagcaaacctaacaggcctccacaagtgtctttaaagtttcataggggtcggtatatgtgattaactgccaaagaagaatattcgccgtgtgtccgaaacgcagtcccctggggcgccacactcggagcctacttgaaccgaagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgcggagtgaggacggtgacagccacgcgcgcgcgtacgcgcccgatgcagcgcggccccgtgaccctagtcggccgctgagaggcgcgcggagtctgggccgctgccgtctaggggtcccgtcccgaggcgtccccggcatctccggcccgaatcccggagtgccgggtcgcgcctgcaccgaaggtcccggctcctgtgccctccctgcagataactggtaagtttagtcttttttgtcttttatttcaggtcccggatccggtggtggtgcaaatcaaagaactgctcctcagtcgatgttgcctttacttctaggcctgtacggaaaccggtgtgttactatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttaataaaagatctttattttcattagatctgtgtgttggttttttgtgtg

SEQ ID NO: 14 - DNA, 720 bp, Artificial Sequence
  Synethetic
  misc_feature: GFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 15 - protein, 595 aa, Artificial Sequence
  Synthetic
  MISC_FEATURE: Merlin protein
MAGAIASRMSFSSLKRKQPKTFTVRIVTMDAEMEFNCEMKWKGKDLFDLVCRTLGLRETWFFGLQYTIKDTVAWLKMDKKVLDHDVSKEEPVTFHFLAKFYPENAEEELVQEITQHLFFLQVKKQILDEKIYCPPEASVLLASYAVQAKYGDYDPSVHKRGFLAQEELLPKRVINLYQMTPEMWEERITAWYAEHRGRARDEAEMEYLKIAQDLEMYGVNYFAIRNKKGTELLLGVDALGLHIYDPENRLTPKISFPWNEIRNISYSDKEFTIKPLDKKIDVFKFNSSKLRVNKLILQLCIGNHDLFMRRRKADSLEVQQMKAQAREEKARKQMERQRLAREKQMREEAERTRDELERRLLQMKEEATMANEALMRSEETADLLAEKAQITEEEAKLLAQKAAEAEQEMQRIKATAIRTEEEKRLMEQKVLEAEVLALKMAEESERRAKEADQLKQDLQEAREAERRAKQKLLEIATKPTYPPMNPIPAPLPPDIPSFNLIGDSLSFDFKDTDMKRLSMEIEKEKVEYMEKSKHLQEQLNELKTEIEALKLKERETALDILHNENSDRGGSSKHNTIKKLTLQSAKSRVAFFEEL

SEQ ID NO: 16 - DNA, 1888 bp, Artificial Sequence
  Synthetic
  misc_feature: NF2-S518A Construct
aaagggctcagagtgcaggccgtggggcgcgagggtcccgggcctgagccccgcgccatggccggggccatcgcttcccgcatgagcttcagctctctcaagaggaagcaacccaagacgttcaccgtgaggatcgtcaccatggacgccgagatggagttcaattgcgagatgaagtggaaagggaaggacctctttgatttggtgtgccggactctggggctccgagaaacctggttctttggactgcagtacacaatcaaggacacagtggcctggctcaaaatggacaagaaggtactggatcatgatgtttcaaaggaagaaccagtcacctttcacttcttggccaaattttatcctgagaatgctgaagaggagctggttcaggagatcacacaacatttattcttcttacaggtaaagaagcagattttagatgaaaagatctactgccctcctgaggcttctgtgctcctggcttcttacgccgtccaggccaagtatggtgactacgaccccagtgttcacaagcggggatttttggcccaagaggaattgcttccaaaaagggtaataaatctgtatcagatgactccggaaatgtgggaggagagaattactgcttggtacgcagagcaccgaggccgagccagggatgaagctgaaatggaatatctgaagatagctcaggacctggagatgtacggtgtgaactactttgcaatccggaataaaaagggcacagagctgctgcttggagtggatgccctggggcttcacatttatgaccctgagaacagactgacccccaagatctccttcccgtggaatgaaatccgaaacatctcgtacagtgacaaggagtttactattaaaccactggataagaaaattgatgtcttcaagtttaactcctcaaagcttcgtgttaataagctgattctccagctatgtatcgggaaccatgatctatttatgaggagaaggaaagccgattctttggaagttcagcagatgaaagcccaggccagggaggagaaggctagaaagcagatggagcggcagcgcctcgctcgagagaagcagatgagggaggaggctgaacgcacgagggatgagttggagaggaggctgctgcagatgaaagaagaagcaacaatggccaacgaagcactgatgcggtctgaggagacagctgacctgttggctgaaaaggcccagatcaccgaggaggaggcaaaacttctggcccagaaggccgcagaggctgagcaggaaatgcagcgcatcaaggccacagcgattcgcacggaggaggagaagcgcctgatggagcagaaggtgctggaagccgaggtgctggcactgaagatggctgaggagtcagagaggagggccaaagaggcagatcagctgaagcaggacctgcaggaagcacgcgaggcggagcgaagagccaagcagaagctcctggagattgccaccaagcccacgtacccgcccatgaacccaattccagcaccgttgcctcctgacataccaagcttcaacctcattggtgacagcctgtctttcgacttcaaagatactgacatgaagcggcttgccatggagatagagaaagaaaaagtggaatacatggaaaagagcaagcatctgcaggagcagctcaatgaactcaagacagaaatcgaggccttgaaactgaaagagagggagacagctctggatattctgcacaatgagaactccgacaggggtggcagcagcaagcacaataccattaaaaagctcaccttgcagagcgccaagtcccgagtggccttctttgaagagctctagcaggtgacccagccaccccaggacctgccacttctcctgctac

SEQ ID NO: 17 - DNA, 2483 bp, Artificial Sequence
  Synthetic
  misc_feature: NF2-400bp- NF2S518A Expression cassette
  misc_feature (1)..(400): NF2 400bp Promoter
  misc_feature (413)..(512): SV40 Intron
  misc_feature (530)..(2137): NF2S518A
  misc_feature (2139)..(2417): NF2S518A
  misc_feature (2435)..(2483): Poly A
aagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgcctgcagataactggtaagtttagtcttttttgtcttttatttcaggtcccggatccggtggtggtgcaaatcaaagaactgctcctcagtcgatgttgcctttacttctaggcctgtacggaaaccggtaaagggctcagagtgcaggccgtggggcgcgagggtcccgggcctgagccccgcgccatggccggggccatcgcttcccgcatgagcttcagctctctcaagaggaagcaacccaagacgttcaccgtgaggatcgtcaccatggacgccgagatggagttcaattgcgagatgaagtggaaagggaaggacctctttgatttggtgtgccggactctggggctccgagaaacctggttctttggactgcagtacacaatcaaggacacagtggcctggctcaaaatggacaagaaggtactggatcatgatgtttcaaaggaagaaccagtcacctttcacttcttggccaaattttatcctgagaatgctgaagaggagctggttcaggagatcacacaacatttattcttcttacaggtaaagaagcagattttagatgaaaagatctactgccctcctgaggcttctgtgctcctggcttcttacgccgtccaggccaagtatggtgactacgaccccagtgttcacaagcggggatttttggcccaagaggaattgcttccaaaaagggtaataaatctgtatcagatgactccggaaatgtgggaggagagaattactgcttggtacgcagagcaccgaggccgagccagggatgaagctgaaatggaatatctgaagatagctcaggacctggagatgtacggtgtgaactactttgcaatccggaataaaaagggcacagagctgctgcttggagtggatgccctggggcttcacatttatgaccctgagaacagactgacccccaagatctccttcccgtggaatgaaatccgaaacatctcgtacagtgacaaggagtttactattaaaccactggataagaaaattgatgtcttcaagtttaactcctcaaagcttcgtgttaataagctgattctccagctatgtatcgggaaccatgatctatttatgaggagaaggaaagccgattctttggaagttcagcagatgaaagcccaggccagggaggagaaggctagaaagcagatggagcggcagcgcctcgctcgagagaagcagatgagggaggaggctgaacgcacgagggatgagttggagaggaggctgctgcagatgaaagaagaagcaacaatggccaacgaagcactgatgcggtctgaggagacagctgacctgttggctgaaaaggcccagatcaccgaggaggaggcaaaacttctggcccagaaggccgcagaggctgagcaggaaatgcagcgcatcaaggccacagcgattcgcacggaggaggagaagcgcctgatggagcagaaggtgctggaagccgaggtgctggcactgaagatggctgaggagtcagagaggagggccaaagaggcagatcagctgaagcaggacctgcaggaagcacgcgaggcggagcgaagagccaagcagaagctcctggagattgccaccaagcccacgtacccgcccatgaacccaattccagcaccgttgcctcctgacataccaagcttcaacctcattggtgacagcctgtctttcgacttcaaagatactgacatgaagcggcttgccatggagatagagaaagaaaaagtggaatacatggaaaagagcaagcatctgcaggagcagctcaatgaactcaagacagaaatcgaggccttgaaactgaaagagagggagacagctctggatattctgcacaatgagaactccgacaggggtggcagcagcaagcacaataccattaaaaagctcaccttgcagagcgccaagtcccgagtggccttctttgaagagctctagcaggtgacccagccaccccaggacctgccacttctcctgctactgtacaagtaaaagcttaataaaagatctttattttcattagatctgtgtgttggttttttgtgtg

SEQ ID NO: 18 - DNA, 2687 bp, Artificial Sequence
  Synthetic
  misc_feature: NF2-610bp- NF2S518A Expression cassette
  misc_feature (1)..(610): NF2 610bp Promoter
  misc_feature (617)..(716): SV40 Intron
  misc_feature (734)..(2341): NF2S518A
  misc_feature (2343)..(2621): NF2S518A
  misc_feature (2639)..(2687): Poly A
aagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgcggagtgaggacggtgacagccacgcgcgcgcgtacgcgcccgatgcagcgcggccccgtgaccctagtcggccgctgagaggcgcgcggagtctgggccgctgccgtctaggggtcccgtcccgaggcgtccccggcatctccggcccgaatcccggagtgccgggtcgcgcctgcaccgaaggtcccggctcctgtgccctccctgcagataactggtaagtttagtcttttttgtcttttatttcaggtcccggatccggtggtggtgcaaatcaaagaactgctcctcagtcgatgttgcctttacttctaggcctgtacggaaaccggtaaagggctcagagtgcaggccgtggggcgcgagggtcccgggcctgagccccgcgccatggccggggccatcgcttcccgcatgagcttcagctctctcaagaggaagcaacccaagacgttcaccgtgaggatcgtcaccatggacgccgagatggagttcaattgcgagatgaagtggaaagggaaggacctctttgatttggtgtgccggactctggggctccgagaaacctggttctttggactgcagtacacaatcaaggacacagtggcctggctcaaaatggacaagaaggtactggatcatgatgtttcaaaggaagaaccagtcacctttcacttcttggccaaattttatcctgagaatgctgaagaggagctggttcaggagatcacacaacatttattcttcttacaggtaaagaagcagattttagatgaaaagatctactgccctcctgaggcttctgtgctcctggcttcttacgccgtccaggccaagtatggtgactacgaccccagtgttcacaagcggggatttttggcccaagaggaattgcttccaaaaagggtaataaatctgtatcagatgactccggaaatgtgggaggagagaattactgcttggtacgcagagcaccgaggccgagccagggatgaagctgaaatggaatatctgaagatagctcaggacctggagatgtacggtgtgaactactttgcaatccggaataaaaagggcacagagctgctgcttggagtggatgccctggggcttcacatttatgaccctgagaacagactgacccccaagatctccttcccgtggaatgaaatccgaaacatctcgtacagtgacaaggagtttactattaaaccactggataagaaaattgatgtcttcaagtttaactcctcaaagcttcgtgttaataagctgattctccagctatgtatcgggaaccatgatctatttatgaggagaaggaaagccgattctttggaagttcagcagatgaaagcccaggccagggaggagaaggctagaaagcagatggagcggcagcgcctcgctcgagagaagcagatgagggaggaggctgaacgcacgagggatgagttggagaggaggctgctgcagatgaaagaagaagcaacaatggccaacgaagcactgatgcggtctgaggagacagctgacctgttggctgaaaaggcccagatcaccgaggaggaggcaaaacttctggcccagaaggccgcagaggctgagcaggaaatgcagcgcatcaaggccacagcgattcgcacggaggaggagaagcgcctgatggagcagaaggtgctggaagccgaggtgctggcactgaagatggctgaggagtcagagaggagggccaaagaggcagatcagctgaagcaggacctgcaggaagcacgcgaggcggagcgaagagccaagcagaagctcctggagattgccaccaagcccacgtacccgcccatgaacccaattccagcaccgttgcctcctgacataccaagcttcaacctcattggtgacagcctgtctttcgacttcaaagatactgacatgaagcggcttgccatggagatagagaaagaaaaagtggaatacatggaaaagagcaagcatctgcaggagcagctcaatgaactcaagacagaaatcgaggccttgaaactgaaagagagggagacagctctggatattctgcacaatgagaactccgacaggggtggcagcagcaagcacaataccattaaaaagctcaccttgcagagcgccaagtcccgagtggccttctttgaagagctctagcaggtgacccagccaccccaggacctgccacttctcctgctactgtacaagtaaaagcttaataaaagatctttattttcattagatctgtgtgttggttttttgtgtg

SEQ ID NO: 19 - DNA, 4174 bp, Artificial Sequence
  Synthetic
  misc_feature: NF2-2.1kb- NF2S518A Expression cassette
  misc_feature (1)..(2091): NF2 2.1kb Promoter
  misc_feature (2104)..(2203): SV40 Intron
  misc_feature (2221)..(3828): NF2S518A
  misc_feature (3830)..(4108): NF2S518A
  misc_feature (4126)..(4174): Poly A
cccggtgtcacgtatttttatcagcagccatttgttgtccctgtggggagcccagaaaacccagcccggttctcccacctgcggggaagaaatgaagcaatgataatgaagacactcccatgccaagcccatactgccctgagcaatcagttttggtggggagaatttgctagcagaaggcaggacataaagtccccacagcagcctcactgggtgtggcagcaaatcattcctgaggcctggccggtgctaccaggcacagcaggggtccgtgcagaagaactagatttattccattaatataccgttacagcaacaatgtctgctgagtactccggaactgtaattcacatttcctgaggctgtagtagggagaatttggaagcagggcagcacagtgctaaccagcaggctctgggtgggacagacctgggtttgagtcctagctttgcctttcactatctgcattatagtcagcgagtggctctatcatatctctcagtatcttcacctgtggagttgggggggtgatgctaatgttagctaacatttatggggtgattattgttgttaaaactcagtacaccgggcgcggtggctcacgcctgtaatcccagcactttgggaggccaaggtgggcagatcacctgaggtcagaagtttgagaccagcctggctaacatggtgaaaccccgtccctactaaaaatacaaaaattagccgggcgtggtggcgggtgcctgtaatcccaactactggggaggctgaggcaggagaattgcttgaacccgggaggcggaggttgcagagagccgagaccgccccattgtactccagcctgggcaacaagagcgaaactctgtctcaaaacaaaaaacaaacgaaaaaacacagtaaattgtccctatttgactactaagagctattttaaaatagaatttattatctaagtacagaaaggtgtaattatcccccctacacccgttagaacccttgatgacactaaataaataatacatgaatacggttcaaaaatgggcatcatagagtataaaataaagaatgctttcattttcaacccaaattcagcaatttcataggatgccaggctcagctctaaagccttatatgtattcagtgcgctgaaattcaacaacctcacgaggtaggtataaccaataataattctaggcggggcgtggtggctcacgcctgtaatcctagcacttcgggaggccgaggcgggcggatcacctgaggttcgagaccagcctgacgaacatggagaaaccccgtctctaataaaaatacaaaattagccgggcgtggtgacgcatacctgtaatcccagctactcgggaggctgacgccggaaaatcgcttgaacccgggaggcggaggttgcagtgagccgagatcgcgccactgcactccagcctgggcaacaagagcgaaactccgtcccaataataataataattaataataataataataacaataataataataattctaaagggaagcagagggatctggcgggaaagtgccagcaaacctaacaggcctccacaagtgtctttaaagtttcataggggtcggtatatgtgattaactgccaaagaagaatattcgccgtgtgtccgaaacgcagtcccctggggcgccacactcggagcctacttgaaccgaagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgcctgcagataactggtaagtttagtcttttttgtcttttatttcaggtcccggatccggtggtggtgcaaatcaaagaactgctcctcagtcgatgttgcctttacttctaggcctgtacggaaaccggtaaagggctcagagtgcaggccgtggggcgcgagggtcccgggcctgagccccgcgccatggccggggccatcgcttcccgcatgagcttcagctctctcaagaggaagcaacccaagacgttcaccgtgaggatcgtcaccatggacgccgagatggagttcaattgcgagatgaagtggaaagggaaggacctctttgatttggtgtgccggactctggggctccgagaaacctggttctttggactgcagtacacaatcaaggacacagtggcctggctcaaaatggacaagaaggtactggatcatgatgtttcaaaggaagaaccagtcacctttcacttcttggccaaattttatcctgagaatgctgaagaggagctggttcaggagatcacacaacatttattcttcttacaggtaaagaagcagattttagatgaaaagatctactgccctcctgaggcttctgtgctcctggcttcttacgccgtccaggccaagtatggtgactacgaccccagtgttcacaagcggggatttttggcccaagaggaattgcttccaaaaagggtaataaatctgtatcagatgactccggaaatgtgggaggagagaattactgcttggtacgcagagcaccgaggccgagccagggatgaagctgaaatggaatatctgaagatagctcaggacctggagatgtacggtgtgaactactttgcaatccggaataaaaagggcacagagctgctgcttggagtggatgccctggggcttcacatttatgaccctgagaacagactgacccccaagatctccttcccgtggaatgaaatccgaaacatctcgtacagtgacaaggagtttactattaaaccactggataagaaaattgatgtcttcaagtttaactcctcaaagcttcgtgttaataagctgattctccagctatgtatcgggaaccatgatctatttatgaggagaaggaaagccgattctttggaagttcagcagatgaaagcccaggccagggaggagaaggctagaaagcagatggagcggcagcgcctcgctcgagagaagcagatgagggaggaggctgaacgcacgagggatgagttggagaggaggctgctgcagatgaaagaagaagcaacaatggccaacgaagcactgatgcggtctgaggagacagctgacctgttggctgaaaaggcccagatcaccgaggaggaggcaaaacttctggcccagaaggccgcagaggctgagcaggaaatgcagcgcatcaaggccacagcgattcgcacggaggaggagaagcgcctgatggagcagaaggtgctggaagccgaggtgctggcactgaagatggctgaggagtcagagaggagggccaaagaggcagatcagctgaagcaggacctgcaggaagcacgcgaggcggagcgaagagccaagcagaagctcctggagattgccaccaagcccacgtacccgcccatgaacccaattccagcaccgttgcctcctgacataccaagcttcaacctcattggtgacagcctgtctttcgacttcaaagatactgacatgaagcggcttgccatggagatagagaaagaaaaagtggaatacatggaaaagagcaagcatctgcaggagcagctcaatgaactcaagacagaaatcgaggccttgaaactgaaagagagggagacagctctggatattctgcacaatgagaactccgacaggggtggcagcagcaagcacaataccattaaaaagctcaccttgcagagcgccaagtcccgagtggccttctttgaagagctctagcaggtgacccagccaccccaggacctgccacttctcctgctactgtacaagtaaaagcttaataaaagatctttattttcattagatctgtgtgttggttttttgtgtg

SEQ ID NO: 20 - DNA, 4378 bp, Artificial Sequence
  Synthetic
  misc_feature: 2.3kb-NF2S518A Expression cassette
  misc_feature (1)..(2301): NF2 2.3kb Promoter
  misc_feature (2308)..(2407): SV40 Intron
  misc_feature (2425)..(4032): NF2S518A
  misc_feature (4034)..(4312): NF2S518A
  misc_feature (4330)..(4378): Poly A
cccggtgtcacgtatttttatcagcagccatttgttgtccctgtggggagcccagaaaacccagcccggttctcccacctgcggggaagaaatgaagcaatgataatgaagacactcccatgccaagcccatactgccctgagcaatcagttttggtggggagaatttgctagcagaaggcaggacataaagtccccacagcagcctcactgggtgtggcagcaaatcattcctgaggcctggccggtgctaccaggcacagcaggggtccgtgcagaagaactagatttattccattaatataccgttacagcaacaatgtctgctgagtactccggaactgtaattcacatttcctgaggctgtagtagggagaatttggaagcagggcagcacagtgctaaccagcaggctctgggtgggacagacctgggtttgagtcctagctttgcctttcactatctgcattatagtcagcgagtggctctatcatatctctcagtatcttcacctgtggagttgggggggtgatgctaatgttagctaacatttatggggtgattattgttgttaaaactcagtacaccgggcgcggtggctcacgcctgtaatcccagcactttgggaggccaaggtgggcagatcacctgaggtcagaagtttgagaccagcctggctaacatggtgaaaccccgtccctactaaaaatacaaaaattagccgggcgtggtggcgggtgcctgtaatcccaactactggggaggctgaggcaggagaattgcttgaacccgggaggcggaggttgcagagagccgagaccgccccattgtactccagcctgggcaacaagagcgaaactctgtctcaaaacaaaaaacaaacgaaaaaacacagtaaattgtccctatttgactactaagagctattttaaaatagaatttattatctaagtacagaaaggtgtaattatcccccctacacccgttagaacccttgatgacactaaataaataatacatgaatacggttcaaaaatgggcatcatagagtataaaataaagaatgctttcattttcaacccaaattcagcaatttcataggatgccaggctcagctctaaagccttatatgtattcagtgcgctgaaattcaacaacctcacgaggtaggtataaccaataataattctaggcggggcgtggtggctcacgcctgtaatcctagcacttcgggaggccgaggcgggcggatcacctgaggttcgagaccagcctgacgaacatggagaaaccccgtctctaataaaaatacaaaattagccgggcgtggtgacgcatacctgtaatcccagctactcgggaggctgacgccggaaaatcgcttgaacccgggaggcggaggttgcagtgagccgagatcgcgccactgcactccagcctgggcaacaagagcgaaactccgtcccaataataataataattaataataataataataacaataataataataattctaaagggaagcagagggatctggcgggaaagtgccagcaaacctaacaggcctccacaagtgtctttaaagtttcataggggtcggtatatgtgattaactgccaaagaagaatattcgccgtgtgtccgaaacgcagtcccctggggcgccacactcggagcctacttgaaccgaagagtccctccctgggccaacgaaggacccaattcccctcgccccggatccagcctctaagtggtttcccgggtaaggtcagcagaggtccgccgcctcaaccagtcttgttagagtaggcacaaaagcattcagagtgactgccactgtagccactcaacgccgagcctaggcctgctttttttgcagttagagtgaaactttccattggtggagtcaccccaaaggaggcgggacggagcgggaaagtcctgcctaccttggccttcccagccaatcgcagcatctatgcgtcagcctccggttcggttgcgctgcgcgtagtggtctgggcgacctgagggcggggccgggagcaggccccgggctgtggggagggtgcgcttcccgcgggcgcgcggagtgaggacggtgacagccacgcgcgcgcgtacgcgcccgatgcagcgcggccccgtgaccctagtcggccgctgagaggcgcgcggagtctgggccgctgccgtctaggggtcccgtcccgaggcgtccccggcatctccggcccgaatcccggagtgccgggtcgcgcctgcaccgaaggtcccggctcctgtgccctccctgcagataactggtaagtttagtcttttttgtcttttatttcaggtcccggatccggtggtggtgcaaatcaaagaactgctcctcagtcgatgttgcctttacttctaggcctgtacggaaaccggtaaagggctcagagtgcaggccgtggggcgcgagggtcccgggcctgagccccgcgccatggccggggccatcgcttcccgcatgagcttcagctctctcaagaggaagcaacccaagacgttcaccgtgaggatcgtcaccatggacgccgagatggagttcaattgcgagatgaagtggaaagggaaggacctctttgatttggtgtgccggactctggggctccgagaaacctggttctttggactgcagtacacaatcaaggacacagtggcctggctcaaaatggacaagaaggtactggatcatgatgtttcaaaggaagaaccagtcacctttcacttcttggccaaattttatcctgagaatgctgaagaggagctggttcaggagatcacacaacatttattcttcttacaggtaaagaagcagattttagatgaaaagatctactgccctcctgaggcttctgtgctcctggcttcttacgccgtccaggccaagtatggtgactacgaccccagtgttcacaagcggggatttttggcccaagaggaattgcttccaaaaagggtaataaatctgtatcagatgactccggaaatgtgggaggagagaattactgcttggtacgcagagcaccgaggccgagccagggatgaagctgaaatggaatatctgaagatagctcaggacctggagatgtacggtgtgaactactttgcaatccggaataaaaagggcacagagctgctgcttggagtggatgccctggggcttcacatttatgaccctgagaacagactgacccccaagatctccttcccgtggaatgaaatccgaaacatctcgtacagtgacaaggagtttactattaaaccactggataagaaaattgatgtcttcaagtttaactcctcaaagcttcgtgttaataagctgattctccagctatgtatcgggaaccatgatctatttatgaggagaaggaaagccgattctttggaagttcagcagatgaaagcccaggccagggaggagaaggctagaaagcagatggagcggcagcgcctcgctcgagagaagcagatgagggaggaggctgaacgcacgagggatgagttggagaggaggctgctgcagatgaaagaagaagcaacaatggccaacgaagcactgatgcggtctgaggagacagctgacctgttggctgaaaaggcccagatcaccgaggaggaggcaaaacttctggcccagaaggccgcagaggctgagcaggaaatgcagcgcatcaaggccacagcgattcgcacggaggaggagaagcgcctgatggagcagaaggtgctggaagccgaggtgctggcactgaagatggctgaggagtcagagaggagggccaaagaggcagatcagctgaagcaggacctgcaggaagcacgcgaggcggagcgaagagccaagcagaagctcctggagattgccaccaagcccacgtacccgcccatgaacccaattccagcaccgttgcctcctgacataccaagcttcaacctcattggtgacagcctgtctttcgacttcaaagatactgacatgaagcggcttgccatggagatagagaaagaaaaagtggaatacatggaaaagagcaagcatctgcaggagcagctcaatgaactcaagacagaaatcgaggccttgaaactgaaagagagggagacagctctggatattctgcacaatgagaactccgacaggggtggcagcagcaagcacaataccattaaaaagctcaccttgcagagcgccaagtcccgagtggccttctttgaagagctctagcaggtgacccagccaccccaggacctgccacttctcctgctactgtacaagtaaaagcttaataaaagatctttattttcattagatctgtgtgttggttttttgtgtg

SEQ ID NO: 21 - DNA, 34 bp, Artificial Sequence
  Synthetic
  misc_feature: NF2 2381 Fw KpnI
ttttttggtacccccggtgtcacgtatttttatc

SEQ ID NO: 22 - DNA, 30 bp, Artificial Sequence
  Synthetic
  misc_feature: NF2 2381 Rv PstI
ttttttctgcagctgcgcgggtccccagcg

SEQ ID NO: 23 - DNA, 34 bp, Artificial Sequence
  Synthetic
  misc_feature: NF2 400 Fw KpnI
ttttttggtaccaagagtccctccctgggccaac

SEQ ID NO: 24 - DNA, 30 bp, Artificial Sequence
  Synthetic
  misc_feature: NF2 400 Rv PstI
ttttttctgcaggcgcgcccgcgggaagcg

SEQ ID NO: 25 - DNA, 31 bp, Artificial Sequence
  Synthetic
  misc_feature: NF2-cDNA-AgeI-Fwd
tttttaccggttaaagggctcagagtgcagg

SEQ ID NO: 26 - DNA, 31 bp, Artificial Sequence
  Synthetic
  misc_feature: NF2-cDNA-BsrGI-Rev
ttttttgtacagtagcaggagaagtggcagg

SEQ ID NO: 27 - DNA, 20 bp, Artificial Sequence
  Synthetic
  misc_feature: Total NF2-Fwd
tgcgagatgaagtggaaagg

SEQ ID NO: 28 - DNA, 20 bp, Artificial Sequence
  Synthetic
  misc_feature: Total NF2-Rev
gccaagaagtgaaaggtgac

SEQ ID NO: 29 - DNA, 20 bp, Artificial Sequence
  Synthetic
  misc_feature: NF2 isoform 1-Fwd
ccttgaaactgaaagagagg

SEQ ID NO: 30 - DNA, 20 bp, Artificial Sequence
  Synthetic
  misc_feature: NF2 isoform 1-Rev
caaggtgagctttttaatgg

SEQ ID NO: 31 - DNA, 20 bp, Artificial Sequence
  Synthetic
  misc_feature: Vector-specific NF2-Fwd
tggtggtgcaaatcaaagaa

SEQ ID NO: 32 - DNA, 18 bp, Artificial Sequence
  Synthetic
  misc_feature: Vector-specific NF2-Rev
cgatcctcacggtgaacg

SEQ ID NO: 33 - DNA, 19 bp, Artificial Sequence
  Synthetic
  misc_feature: FN1-Fwd
caagccagatgtcagaagc

SEQ ID NO: 34 - DNA, 19 bp, Artificial Sequence
  Synthetic
  misc_feature: FN1-Rev
ggatggtgcatcaatggca

SEQ ID NO: 35 - DNA, 19 bp, Artificial Sequence
  Synthetic
  misc_feature: Col3a1-Fwd
ccctggatctccaggatac

SEQ ID NO: 36 - DNA, 19 bp, Artificial Sequence
  Synthetic
  misc_feature: Col3a1-Rev
gtccgggtctacctgattc

SEQ ID NO: 37 - DNA, 24 bp, Artificial Sequence
  Synthetic
  misc_feature: KROX20-Fwd
caccagctgtctgacaacatctac

SEQ ID NO: 38 - DNA, 20 bp, Artificial Sequence
  Synthetic
  misc_feature: KROX20-Rev
cctgcacagccagaataagg

SEQ ID NO: 39 - DNA, 23 bp, Artificial Sequence
  Synthetic
  misc_feature: MBP-Fwd
ttaagctgggaggaagagatagt

SEQ ID NO: 40 - DNA, 20 bp, Artificial Sequence
  Synthetic
  misc_feature: MBP-Rev
gggaggggtcatctgctcta

SEQ ID NO: 41 - DNA, 23 bp, Artificial Sequence
  Synthetic
  misc_feature: GAPDH-Fwd
caactttggtatcgtggaaggac

SEQ ID NO: 42 - DNA, 21 bp, Artificial Sequence
  Synthetic
  misc_feature: GAPDH-Rev
acagtcttctggatggcagtg

SEQ ID NO: 43 - DNA, 23 bp, Artificial Sequence
  Synthetic
  misc_feature: RPL13A-Fwd
cctggaggagaagaggaaagaga

SEQ ID NO: 44 - DNA, 25 bp, Artificial Sequence
  Synthetic
  misc_feature: RPL13A-Rev
ttgaggacctctgtgtatttgtcaa

SEQ ID NO: 45 - DNA, 23 bp, Artificial Sequence
  Synthetic
  misc_feature: RPL13A-Fwd
cctggaggagaagaggaaagaga

SEQ ID NO: 46 - DNA, 25 bp, Artificial Sequence
  Synthetic
  misc_feature: RPL13A-Rev
ttgaggacctctgtgtatttgtcaa

SEQ ID NO: 47 - DNA, 21 bp, Artificial Sequence
  Synthetic
  misc_feature: Target sequence in NF2 gene
ggatgaagctgaaatggaata